(19) United States
(12) Patent Application Publication
(10) Pub. No.: US 2025/0270530 A1
(43) Pub. Date: Aug. 28, 2025

(54) Methods and compositions for endogenous exon splicing using dCas13-RBM25 fusions

(71) Applicant: THE GOVERNING COUNCIL OF THE UNIVERSITY OF TORONTO, Toronto (CA)

(72) Inventors: Benjamin BLENCOWE, Toronto (CA); Jack Daiyang LI, Toronto (CA); Miko Joonas Oskari TAIPALE, Toronto (ON)

(21) Appl. No.: 19/064,468

(22) Filed: Feb. 26, 2025

(30) Foreign Application Priority Data
Feb. 26, 2024 (LU) LU506463

(51) Int. Cl. C12N 9/22; C07K 14/435; C12N 15/11

(52) U.S. Cl. CPC C12N 9/226; C07K 14/435; C12N 15/111; C07K 2319/09; C07K 2319/85; C12N 2310/20

(57) ABSTRACT

The present disclosure provides fusion proteins comprising a catalytically deactivated Cas13d and the splicing factor RBM25. The present disclosure also provides isolated nucleic acids encoding the fusion proteins and cells comprising said nucleic acids. The present disclosure further provides methods for modulating alternative splicing.

### RELATED APPLICATION

This application claims benefit of Luxembourg Patent Application no. LU506463 filed Feb. 26, 2024, incorporated herein by reference in its entirety.

### INCORPORATION OF SEQUENCE LISTING

An XML Sequence Listing “SL_2223-P73603US00.xml” (13.4 KB), submitted via EFS-WEB and created on Feb. 28, 2024, is herein incorporated by reference.

### FIELD

The present disclosure relates to fusion proteins and methods for modulating endogenous exon splicing and particularly to dCAS13-RBM25 fusions and methods of use thereof.

### BACKGROUND

Alternative splicing is a process that allows a single pre-mRNA to produce multiple isoforms by selective usage of splice sites. It is widespread in mammals and greatly expands transcriptomic and proteomic diversity¹⁻³. Consequently, it plays important roles in fundamental processes such as development, behaviour, signaling, and immune responses⁴⁻⁷. Moreover, the misregulation of alternative splicing causes or contributes to numerous human diseases, in particular cancers and brain disorders⁶⁻⁹.

Given its importance, methods for the efficient and precise manipulation of alternative isoforms are needed to uncover their biological functions, as well as to correct their misregulation in diseases. Antisense oligonucleotides (ASOs) that target cis-elements in pre-mRNA have shown efficacy in both the repression (e.g by targeting splice sites) and activation (e.g by targeting splicing silencer elements) of alternative exons, and currently are used in the clinic^{10,11}. However, ASOs are transient in their effect and require laborious tiling experiments to determine effective sequences, making them unsuitable for high-throughput interrogation of exon function. Moreover, efficient splicing activation via typical ASOs requires that an exon of interest is under control of a proximal repressor element that can be specifically blocked, which limits the generalizability of this approach.

Other methods for the modulation of alternative splicing include targeted deletion of exons using single and pairs of CRISPR-Cas nucleases^{12,13}, engineered Pumilio RNA binding domains fused to splicing regulatory sequences (PUF-ESFs)^{14,15}, exon-targeting modified U1 snRNAs (ExSpeU1s)¹⁶, isoform-specific knockdown using RNAi¹⁷, and small molecules¹⁸. These approaches all have inherent limitations, such as the inability to activate exons (CRISPR-Cas genomic deletion), reduced target specificity (PUFs-ESFs), perturbation of overall transcript expression (isoform-specific RNAi and ExSpeU1s), and lack of generalizability for targeting (small molecules). As such, no single method currently affords the targeted repression and activation of endogenous alternative exons in a manner that is efficient, specific, and scalable.

The recent discovery and engineering of RNA-targeting type-VI CRISPR systems have greatly advanced possibilities for directing specific transcriptome perturbations¹⁹. In particular, the CRISPR-Cas13d system from Ruminococcus flavefaciens XPD3002 (CasRx) achieves near-complete RNA knockdown when expressed in human cell lines, and high target specificity has been reported, despite a non-specific bystander cleavage activity of this system in certain contexts²⁰. Importantly, catalytically deactivated (d) CasRx can induce efficient skipping of alternative exons through steric blocking when tethered directly to the exon or to its splice sites²⁰-22. In addition to being the most active dCas13 variant in repressing splicing reported to date, dCasRx further offers the advantages of lacking bystander cleavage activity, lack of dependence on a protospacer adjacent motif (PAM), the ability to process its own guide (g) RNA array for multiplexed targeting applications, and efficient activity when expressed from stably integrated constructs^{20,22}. It is also smaller in size relative to other Cas13 family members.

However, efficient and generalizable exon activation by recruitment of dCasRx has not been previously demonstrated. Although dCasRx-mediated steric blocking of splicing silencer elements can stimulate exon inclusion in specific cases^{22,23}, this strategy has similar drawbacks as ASOs. To address this issue, fusion of positive splicing factors to dCasRx, followed by gRNA-directed recruitment to the proximal intron, has been proposed as a strategy for versatile exon activation. Previously, fusion of dCasRx to the N-and C-terminal domains of the splicing regulator RBFOX1, substituting for its RNA Recognition Motif (RRM) (RBFOX1N-dCasRx-C), as well as to the splicing factor RBM38 (dCasRx-RBM38), was shown to promote the inclusion of SMN2 exon 7 when tethered to its downstream intronic sequences in transcripts expressed from transfected minigene reporters²¹. Efficient activation in this case required simultaneous delivery of three intron-targeting gRNAs, and activation of the targeted endogenous exon was considerably less efficient. Moreover, this study focused on an analysis of only two well characterized, pre-selected splicing factors tethered to a single splicing reporter. Related to the study by Du et al²¹, US Patent Application No. 2021/388351 demonstrated pools of three gRNAs showed the highest fold change of inclusion/exclusion ratio of SMN2-E7 activation. Similarly, US Patent Application No. 2021/388351 demonstrated the requirement of three gRNAs for dCasRx-DAZAP1, three gRNAs for SNRPC-dCasRx and two gRNAs for RBM38-dCasRx. In fact, both Du et al., and US Patent Application No. 2021/388351 suggest that fewer than 3 gRNAs are not sufficient. There remains a need for an efficient, specific, and versatile dCasRx-based artificial splicing factor for the targeted control of endogenous alternative exons.

### SUMMARY

In one aspect is provided a method of modulating splicing in a cell, comprising introducing into the cell:
a fusion protein comprising a catalytically deactivated Cas13d (dCas13d) and a splicing factor, wherein the splicing factor is RBM25 polypeptide, or a nucleic acid or vector encoding said fusion protein; and
one or more guide RNAs,
wherein the one or more guide RNAs targets an intron downstream of an exon of a first pre-mRNA.

Another aspect relates to a fusion protein comprising a catalytically deactivated Cas13d (dCas13d) and a splicing factor, wherein the splicing factor is RBM25 polypeptide.

Other aspects include, a nucleic acid encoding the fusion protein, a vector comprising the nucleic acid, a recombinant cells expression the fusion protein and/or comprising the nucleic acid, a composition and/or a kit comprising any of the foregoing.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIGS. 1A-1D are graphs and images showing the development of dual-luciferase reporters for assaying dCasRx-SF modulation of splicing. (A) Schematics of dCasRx-SF tethering assay and dual-luciferase splicing reporters based on human SMN2 exon 7 and mouse Mef2d microexon. Both reporters contain full endogenous sequences for the upstream constitutive exon (C1) and the flanking introns, whereas the downstream constitutive exon (C2) is truncated to 39nt (for Mef2d) and 12nt (for SMN2) to avoid introducing stop codons upstream of the Nano luciferase (NLuc) coding sequence. A 1nt insertion was engineered in the Mef2d microexon, such that exon inclusion or skipping results in expression of Firefly luciferase (Fluc) or NLuc, respectively. For the SMN2 reporter, a native stop codon within exon 7 terminates translation upstream of the NLuc coding sequence, resulting in only FLuc expression upon exon inclusion, while both NLuc and FLuc are expressed upon skipping. The SMN2 reporter is driven by a constitutive CMV promoter, while the Mef2d reporter is driven by a dox-inducible TRE3G promoter for use in N2A Flp-In cells that express rTetR. (B) RT-PCR splicing assay (left) and luciferase log2FC values (right) of the SMN2 luciferase reporter following co-transfection into HEK293T cells of dCasRx or RBFOX1N-dCasRx-C with NT gRNA, a single gRNA targeting the downstream intron (DN2), or three gRNAs targeting the downstream intron (DN1-3). P-values are derived from two-sided Wilcoxon tests (n=12). (C) Optimization of guide (g) RNA targeting to the Mef2d dual-luciferase splicing reporter. Each dCasRx-SF construct is co-transfected with one of 23 gRNAs that tile across the intronic and exonic sequences proximal to the Mef2d microexon, or a non-targeting (NT) gRNA. For each construct and gRNA combination, the luciferase fold change (FC) is calculated as the FLuc/NLuc ratio of the tethered (targeting) condition divided by that of the non-tethered (NT) condition. FC values greater than one indicate exon activation, whereas values less than one indicate exon repression. (D) RT-PCR splicing assay (left) and luciferase log2FC values (right) of the Mef2d luciferase reporter following co-transfection of dCasRx or RBFOX1N-dCasRx-C with NT gRNA, a single gRNA targeting the downstream intron (DN), or a single gRNA targeting the exon (EX) in N2A Flp-In cells. P-values are derived as in (C) (n=12).

FIGS. 2A-2H are plots and images that show a systematic screen of dCasRx-SFs identifying dCasRx-RBM25 as an efficient splicing activator. (A) Luciferase log2FC scores for each dCasRx-SF construct when tethered to the SMN2 exon 7 splicing reporter in HEK293T cells using a single downstream-targeting gRNA (DN2). Constructs are ranked by their log2FC values, and the log2FC of dCasRx alone is indicated with a dotted line. The density distribution of log2FC values for all tested constructs is shown on the right. Constructs scoring above log2FC>0.25 at FDR<0.1 are considered significant and highlighted in red. (B) Luciferase log2FC scores for each dCasRx-SF construct when tethered against the Mef2d microexon reporter in N2A Flp-In cells using a single downstream-targeting gRNA (DN). Constructs are ranked by their log2FC value. The log2FC value of dCasRx alone is indicated with a dotted line. The density distribution of log2FC values for all tested constructs is shown to the right. Constructs scoring above log2FC>1 at FDR<0.1 are considered significant. (C) Overlap of significant activators from the SMN2 triple-gRNA screen and the Mef2d single-gRNA screens. P-value from hypergeometric test. (D) Scatterplots showing correlation between measurements of luciferase log2FC and RT-PCR changes in percent spliced in (dPSI) in response to expression of selected dCasRx-SF constructs tethered to the Mef2d (top panel) or SMN2 (bottom panel) dual luciferase reporters. (E) RT-PCR assays monitoring splicing of the SMN2 exon 7 dual luciferase reporter, following co-transfection of RBFOX1N-dCasRx-C or dCasRx-RBM25 with indicated gRNA constructs. (F) RT-PCR assays monitoring splicing of the pCI-SMN2 exon 7 minigene26, following co-transfection of RBFOX1N-dCasRx-C or dCasRx-RBM25 with indicated gRNA constructs. (G) RT-PCR assays monitoring splicing of the Mef2d microexon dual luciferase reporter, following co-transfection of RBFOX1N-dCasRx-C or dCasRx-RBM25 with indicated gRNAs. (H) Western blot using an anti-HA antibody to compare protein expression levels of dCasRx constructs 48 h after transfection into HEK293T cells. Detection of Tubulin is used as loading control.

FIGS. 3A-3G are various illustrations relating to the systematic screen of dCasRx-SFs identifying dCasRx-RBM25 as an efficient splicing activator. (A) Summary of human splicing factors grouped by annotation source. Coverage is calculated as the number of proteins successfully fused to dCasRx divided by the total number of proteins in the annotation category. (B) Luciferase log2FC scores for each dCasRx-SF construct when tethered to SMN2 exon 7 splicing reporter mRNA in HEK293T cells using three downstream-targeting gRNA (DN1-3). Constructs are ranked by their log2FC values, and the log2FC of dCasRx alone is indicated with a dotted line. The density distribution of log2FC values for all tested constructs is shown on the right. Constructs scoring above log2FC>0.4 at FDR<0.1 are considered significant. (C) Gene Ontology (GO) analysis of SMN2triple-gRNA screen hits versus the background of all genes represented in the dCasRx-SF tethering library. (D) Venn diagram depicting overlap of spliceosomal E complex proteins (CORUM annotation) and Mef2d activators. P-values from hypergeometric test. (E) Summary of all yeast U1 snRNP proteins, their human homologs, and whether they are hits in either the SMN2 or Mef2d splicing reporter screens. (F) Scatterplot showing correlation of the size of the protein (number of amino acids) fused to dCasRx versus its luciferase Log2FC score for the Mef2d reporter screen. (G) Scatterplot showing correlation of the size of the protein (number of amino acids) fused to dCasRx versus its luciferase Log2FC score for the SMN2 triple-gRNA reporter screen.

FIGS. 4A-4D are graphs and images showing assays of deletion derivatives of dCasRx-RBM25. (A) Schematic depicting RBM25 protein domains (bottom) and regions of RBM25 deleted in mutants analyzed in FIGS. 4B-4D (top). (B) RT-PCR assays monitoring splicing of pCI-SMN2 minigene following co-transfection of each dCasRx-RBM25 mutant with either non-targeting (NT) or single downstream-targeting gRNA (DN2) in HEK293T cells. (C) Luciferase fold-change of the Mef2d reporter following co-transfection of each dCasRx-RBM25 mutant with either NT or a downstream-targeting gRNA in N2A Flp-In cells. (D) Western blot using anti-HA antibody to detect expression of transfected dCasRx-RBM25 constructs in HEK293T cells. Tubulin is used as loading control.

FIGS. 5A-5D are graphs and images showing dCasRx-RBM25 promotes efficient bidirectional modulation of endogenous alternative exons. (A) Boxplots comparing exon activation efficiency of downstream intron-targeted dCasRx constructs for 16 endogenous alternative exons analyzed in HEK293T cells (top right). A two-sided paired Wilcoxon test was used to assess significance. Barplots showing activation efficiency for each targeted exon between dCasRx constructs (bottom). For each exon, the gRNA mediating the most efficient activation was used to calculate dPSI values relative to a non-targeting control. (B) RT-PCR assays (top) and corresponding quantification (bottom) of MAPT exon 10 PSI levels in HEK293T cells co-transfected with indicated dCasRx and downstream intron-targeting gRNA constructs. (C) RT-PCR assays (top) and corresponding quantification (bottom) of CPEB4 microexon PSI levels in HEK293T cells co-transfected with indicated dCasRx and downstream intron-targeting gRNA constructs. An additional alternative exon adjacent to the microexon of interest, gives rise to four potential isoforms, as indicated. (D) RT-PCR assays (top) and corresponding quantification (bottom) of repression efficiency of exonic-targeted dCasRx constructs across three endogenous alternative exons in HEK293T cells. The relative inclusion level for each dCasRx construct is calculated from dividing the PSI value of the targeting condition by the PSI of the non-targeting condition.

FIG. 6 is a series of images and graphs showing the results of RT-PCR assays (top) and corresponding quantifications (bottom) for 12 endogenous alternative exons targeted by indicated dCasRx constructs and gRNAs in HEK293T cells.

FIG. 7 is a series of graphs showing the results of RT-qPCR assays with primers amplifying distal, constitutive exons to monitor total gene expression levels in HEK293T cells co-transfected with dCasRx-RBM25 and indicated gRNAs. P-values are calculated from unpaired Student t-tests, with p>0.05 unless otherwise indicated.

FIGS. 8A-8C are plots showing RNA-Seq analyses of global alternative splicing. (A) RNA-seq analysis of global alternative splicing 48 h following co-transfection of constructs expressing dCasRx-RBM25 and a non-targeting (NT) gRNA, or dCasRx with a NT gRNA (left). Differentially spliced exons (absolute dPSI>15 and MV (0.95)>=0.1) are shown as darker grey dots. Dotted lines represent 15% changes in PSI. RNA-seq analysis of global gene expression 48 h following co-transfection dCasRx-RBM25 with NT gRNA, or dCasRx with NT gRNA (right). Differentially expressed genes (log2FC>1, FDR<0.05) are shown as darker grey dots. The expression of RBM25 is indicated with an arrow. (B) RNA-seq analysis of global alternative splicing 48 h following co-transfection of constructs expressing RBFOX1N-dCasRx-C and a non-targeting (NT) gRNA, or dCasRx with a NT gRNA (left). Differentially spliced exons (absolute dPSI>15 and MV (0.95)>=0.1) are shown as darker grey dots. Dotted lines represent 15% changes in PSI. RNA-seq analysis of global gene expression 48 h following co-transfection RBFOX1N-dCasRx-C with NT gRNA, or dCasRx with NT gRNA (right). Differentially expressed genes (log2FC>1, FDR<0.05) are shown as darker grey dots. (C) RNA-seq analysis of global alternative splicing 48 h following co-transfection of dCasRx-RBM25 with NT gRNA, or a gRNA targeting CD46 exon 13 for repression (left). Differentially spliced exons (absolute dPSI>15 and MV (0.95)>=0.1) are shown as darker grey dots. The endogenous CD46 exon 13 is indicated with an arrow. Dotted lines represent 15% changes in PSI. RNA-seq analysis of global gene expression 48 h following co-transfection co-transfection of dCasRx-RBM25 with NT gRNA, or a gRNA targeting CD46 exon 13 for repression (right). Differentially expressed genes (log2FC>1, FDR<0.05) are shown as darker grey dots.

FIGS. 9A-9B are plots showing high on-target specificity of dCasRx-RBM25. (A) RNA-seq analysis of global alternative splicing 48 h following co-transfection of dCasRx-RBM25 with NT gRNA, or a gRNA targeting MAPT exon 10 for activation (left). Differentially spliced exons (absolute dPSI>15 and MV (0.95)>=0.1) are shown as darker grey dots. The endogenous MAPT exon 10 is indicated with an arrow. Dotted lines represent 15% changes in PSI. RNA-seq analysis of global gene expression 48 h following co-transfection co-transfection of dCasRx-RBM25 with NT gRNA, or a gRNA targeting MAPT exon 10 for activation (right). Differentially expressed genes (log2FC>1, FDR<0.05) are shown as darker grey dots. (B) RNA-seq analysis of global alternative splicing 48 h following co-transfection of RBFOX1N-dCasRx-C with NT gRNA, or a gRNA targeting MAPT exon 10 for activation (left). Differentially spliced exons (absolute dPSI>15 and MV (0.95)>=0.1) are shown as darker grey dots. The endogenous MAPT exon 10 is indicated with an arrow. Dotted lines represent 15% changes in PSI. RNA-seq analysis of global gene expression 48 h following co-transfection co-transfection of RBFOX1N-dCasRx-C with NT gRNA, or a gRNA targeting MAPT exon 10 for activation (right). Differentially expressed genes (log2FC>1, FDR<0.05) are shown as darker grey dots.

FIGS. 10A-10D are images showing combinatorial exon modulation with dCasRx-RBM25. (A) Strategy for targeted simultaneous modulation of two endogenous alternative exons using a two-spacer gRNA array. After processing by dCasRx, one spacer targets the exonic sequence of an alternative exon for repression, while the other spacer targets the downstream intronic sequence of a second alternative exon for activation. (B) RT-PCR assays monitoring inclusion levels of endogenous MAPT exon 10 and CD46 exon 13 following transfection of single-targeting gRNAs, or dual-targeting gRNA arrays, with dCasRx-RBM25 constructs. The spacer sequences of each gRNA array are indicated above each lane. (C) RT-PCR assays monitoring inclusion levels of endogenous MEF2D microexon and MDM4 exon 6 following transfection of single-targeting gRNAs, or dual-targeting gRNA arrays, and dCasRx-RBM25 constructs. The spacer sequences of each gRNA array are indicated above each lane. (D) RT-PCR assays monitoring inclusion levels of endogenous SPAG9 exon 24 and PLOD2 exon 14 following transfection of single-targeting gRNAs, or dual-targeting gRNA arrays, and dCasRx-RBM25 constructs. The spacer sequences of each gRNA array are indicated above each lane.

FIGS. 11A-11D are schematics and images for stable cell lines. FIGS. 11A and 11C show schematics for dCasRx-RBM25 and RBFOX1N-dCasRx stable expression constructs, and FIG. 11B shows expression of the expression constructs and FIG. 11D shows RT-PCR splicing analysis following stable co-expression of dCasRx-RBM25 or RBFOX1N-dCasRx-C with gRNAs targeting single exons for activation, or with arrays targeting two or five exons simultaneously. Constructs were stably expressed as shown in by co-transfection of piggybac vectors and antibiotic selection, and were tested in two human cell lines (HEK293T and HAP1).

### DETAILED DESCRIPTION OF THE DISCLOSURE

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. Definitions

As used herein, the terms “peptide,” “polypeptide,” and “protein” refer to any chain of two or more natural or unnatural amino acid residues, regardless of post-translational modifications (e.g., glycosylation or phosphorylation).

The term “functional variant” as used herein includes modifications of the polypeptide sequences disclosed herein that perform substantially the same function as the polypeptide molecules disclosed herein in substantially the same way. For example, the functional variant may comprise sequences having at least 50%, at least 60%, at least 70%, at least 80%, or at least 90%, or at least 95% sequence identity to the sequences disclosed herein provided that the variant retains activity, for example for dCas13 functional variants at least or about the same nucleic acid binding in a gRNA dependent manner or for RBM25 functional variants, at least or about the same amount of splicing activity when fused to dCas13 (or variant) when targeted by a suitable gRNA in a cell. The functional variant may also comprise conservatively substituted amino acid sequences of the sequences disclosed herein or a fragment of any of the sequences described herein. For example, a functional variant of RMB25 may be a deletion mutant or fragment deleted for the C-terminus (e.g. 610-843 aa of dCas Rx-uniprot P49756-1) or the PWI domain (e.g. aa 750-843 of uniprot P49756-1) and optionally which has at least 80% sequence identity to the portion of SEQ ID NO: 4 lacking the C-terminus. Other deletion mutants include for example A558-587, A177-193, A376-392 and A953-966 as well as combinations thereof (see for example PMID: 30241607).

As used herein, the term “linker” refers to any moiety, preferably a stretch of amino acids, that links together different functional domains of a polypeptide. Various linkers are contemplated and the linker can be of any appropriate length and structure.

The terms “nucleic acid” or “oligonucleotide” as used herein means two or more covalently linked nucleotides. Unless the context clearly indicates otherwise, the term generally includes, but is not limited to, deoxyribonucleic acid (DNA) and ribonucleic acid (RNA), which may be single-stranded (ss) or double stranded (ds). For example, the nucleic acid molecules or polynucleotides of the disclosure can be composed of single-and double-stranded DNA, DNA that is a mixture of single-and double-stranded regions, single-and double-stranded RNA, and RNA that is a mixture of single-and double-stranded regions, hybrid molecules comprising DNA and RNA that may be single-stranded or, more typically double-stranded or a mixture of single-and double-stranded regions. The sequences provided herein may be DNA sequences or RNA sequences, however it is to be understood that the provided sequences encompass both DNA and RNA, as well as the complementary RNA and DNA sequences, unless the context clearly indicates otherwise. For example, the sequence 5′-GAATCC-3′, is understood to include 5′-GAAUCC-3′, 5′-GGATTC-3′, and 5′GGAUUC-3′.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=[number of identical overlapping positions]/[total number of positions]×100%). The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The term “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-chain length for one another. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The term “operably linked” as used herein refers to a relationship between two components that allows them to function in an intended manner.

The term “promoter” or “promoter sequence” generally refers to a regulatory DNA sequence capable of being bound by an RNA polymerase to initiate transcription of a downstream (i.e. 3′) sequence to generate an RNA. Suitable promoters may be derived from any organism and may be bound or recognized by any RNA polymerase. Suitable promoters will be known to the skilled person.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 20%, 5-20%, or 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary.

Although any methods and materials similar or equivalent to those described herein can also be used in the practice or testing of the present disclosure, examples of methods and materials are now described.

### II. Fusion Protein

Fusion proteins comprising catalytically deactivated Cas13d from Ruminococcus flavefaciens XPD3002 and splicing factor RBM25 (dCasRx-RBM25) are described herein. Such fusion proteins when expressed in a cell with an intron or exon targeting guide RNA can efficiently activate or repress splicing. For example, as demonstrated therein, when expressed with a guide RNA that targets an intron proximal to an endogenous exon of a pre-mRNA, the exon is activated and at an increased level relative to other fusions, resulting in a higher frequency of the isoform of mature mRNA that includes the exon, compared to for example a fusion protein wherein the dCas13d is fused to the splicing factor RBFOX1 (dCasRx-RBFOX1C). It is also demonstrated that efficient activation of the exon can occur with the co-expression of dCasRx-RBM25 and a single guide RNA. Other splicing factors such RBFOX1C can require additional guide RNAs. For example, it is also demonstrated that efficient exon activation o occurs only when dCasRx-RBFOX1C is co-expressed with three guide RNAs that target the intron downstream of the exon.

As demonstrated herein, the present disclosure demonstrates the identification and characterization of dCasRx-RBM25 as a potent, versatile, and highly specific artificial splicing modulator of target endogenous exons. As indicated in the Examples, dCasRx-RBM25 was identified through a high-throughput screening approach that interrogated>300 human splicing-related proteins fused to dCasRx and tethered to dual-luciferase minigene splicing reporters, one containing a microexon from the Mef2d gene, and the other exon 7 from the SMN2 gene. These reporter exons differ in many properties, including their length, basal inclusion levels, and factors required for their endogenous regulation. Likely reflecting these differences, only five (20%) of the dCasRx activators defined as hits were shared between the two reporter screens. Strikingly, dCasRx-RBM25 emerged as the most robust and consistent activator when tethered using single gRNAs.

The design and application of dCasRx-RBM25 has been recognized as the “most successful RNA-targeting CRISPR-based artificial SFs to date”.⁴⁴ The finding that dCasRx-RBM25 was the most potent gRNA dependent activator of exon splicing was not predictable based on previously used splicing factors, such as RBFOX1N-dCasRx-C and dCasRx-RBM38. RBM25 had been discounted as a viable splicing factor construct. Further, as demonstrated herein, dCasRx-RBM25 outperformed RBFOX1N-dCasRx-C with a single guide RNA in almost all tested contexts, which was entirely unexpected.

Accordingly, in one aspect, provided herein is a fusion protein comprising a catalytically deactivated Cas13d (dCas13d) and a splicing factor, wherein the splicing factor is RBM25 polypeptide.

### dCas13d

CRISPR (clustered regularly interspaced short palindromic repeats)-Cas (CRISPR associated) systems comprise a Cas nuclease and a guide RNA for sequence specificity. Different types of CRISPR-Cas systems are known. For example, type II includes the nuclease Cas9, characterized by two nuclease domains (a HNH domain and a RuvC domain) and is commonly used in gene editing. Type VI includes Cas13, characterized by the nuclease domain HEPN and self-processing of CRISPR RNA (crRNA) into mature guide RNAs. Cas13 has been classified into different families, including Cas13d (20).

As used herein, the term “Cas13d” refers to a family of Type VI CRISPR ribonucleases characterized by two HEPN nuclease domains and encompasses Cas13d orthologs from distinct bacterial species.

Cas13d having a sequence as shown in the Table of Sequences herein or a functional variant thereof is preferred.

In some embodiments, the Cas13d is Cas13d from Ruminococcus flavefaciens strain XPD3002.

As used herein, the term “catalytically deactivated Cas13d”, “deactivated Cas13d” or “dCas13d” refers to Cas13d whose nuclease activity is reduced by at least 95% compared to the same Cas13d, or optionally abolished (e.g. no detectable activity or decreased greater than 99%).

Cas13d may be deactivated by, for example, introducing a mutation in the sequence encoding the nuclease domain of Cas13d.

In some embodiments, the catalytically deactivated Cas13d is deactivated Cas13d from Ruminococcus flavefaciens strain XPD3002 (dCasRx) also referred to as dRfxCas13d, or a functional variant thereof. For example, Cas13d catalytic activity of CasRx can be deactivated by introducing mutations R239A/H244A/R858A/H863A into the sequence of CasRx.

In some embodiments, the catalytically deactivated Cas13d comprises an amino acid sequence of at least 80%, at least 85%, at least 90%, or at least 95% sequence identity with SEQ ID NO: 3. In some embodiments, the catalytically deactivated Cas13d comprises an amino acid sequence of at least 90% sequence identity with SEQ ID NO: 3. In some embodiments, the catalytically deactivated Cas13d comprises an amino acid sequence of at least 90% sequence identity with SEQ ID NO: 3. In some embodiments, the catalytically deactivated Cas13d comprises an amino acid sequence of SEQ ID NO: 3.

The dCas13d of the fusion protein disclosed herein can be linked to a nuclear localization signal (NLS), for example it can be linked directly or via a linker. The fusion protein can comprise one or more NLS. For example it can comprise a NLS fused to the N terminus and/or a NLS fused to the C terminus of dCas13d. Any suitable NLS may be used. For example, a NLS with the sequence PKKKRKV (SEQ ID NO: 5) or PAAKRVKLD (SEQ ID NO: 6) may be used. Both SEQ ID NOs: 5 and 6, for example, have been incorporated for dCasRx-RBM25 activation of the Mef2d reporter.

In some embodiments, the dCas13d is linked to a NLS comprising an amino acid sequence of SEQ ID NO: 5. In some embodiments, the dCas13d is linked to a NLS comprising an amino acid sequence of SEQ ID NO: 6. In some embodiments, the dCas13d is linked to a first NLS comprising an amino acid sequence of SEQ ID NO: 5 and a second NLS comprising an amino acid sequence of SEQ ID NO: 6.

In some embodiments, the catalytically deactivated Cas13d (dCas13d) is deactivated Cas13d from Ruminococcus flavefaciens strain XPD3002 (dCasRx).

### Splicing Factor

RBM25 is an RNA binding protein and comprises an RNA Recognition Motif (RRM), arginine/glutamic acid/aspartate (RED)-rich domain, and nucleic acid binding PWI motif, interspersed with intrinsically disordered regions. Previous affinity purification coupled to mass spectrometry experiments showed that RBM25 interacts with various splicing factors, including multiple hnRNPs, SR proteins, the U2 snRNP SF3 complex, and U1 snRNP components. Deletion of the RED domain or the and N-terminal region (which includes the RRM) resulted in abrogation of most interactions, whereas PWI deletion selectively affected interaction with the SF3 complex (Carlson et al, 2017). It is demonstrated herein that relative to full-length dCasRx-RBM25, deletion of each region, to varying extents, results in a loss of splicing activity for different reporters, with the N-terminus, RRM, and RED deletions drastically reducing activity, and the PWI deletion resulting in a minor reduction. Without wishing to be bound by a theory, the high degree of splicing activation observed for tethered dCasRx-RBM25 may be due to its interaction with numerous splicing factors involved in exon and intron definition at early stages of spliceosome assembly.

As used herein, the term “RBM25 polypeptide” refers to RNA binding motif protein 25 and encompasses functional variants, including isoforms, and mutant forms as well as fragments of any thereof that retain splicing activity when fused to dCas13d and when targeted to a premRNA. The term also encompasses homologues in different species and sequences with at least 80%, at least 85%, at least 90%, at least 95% sequence identity to SEQ ID NO: 4. For example, the RBM25 can comprise the sequence as described in UniProt Accession No. P49756-1.

In some embodiments, the fusion protein disclosed herein comprises RBM25 polypeptide, optionally RBM25, or a functional variant, or a fragment of any thereof.

The functional variants of RBM25 that can be used in the fusion protein of the present disclosure are for example conservatively substituted sequences and/or fragments that are capable of interacting with the same or substantially the same splicing factors as the full-length RBM25.

In some embodiments, the RBM25 polypeptide comprises an amino acid sequence of at least 90%, or at least 95% sequence identity with SEQ ID NO: 4. In some embodiments, the RBM25 polypeptide is a functional variant of RBM25 that comprises an amino acid sequence of at least 90% sequence identity with SEQ ID NO: 4. In some embodiments, the RBM25 or functional variant thereof comprises an amino acid sequence of at least 95% sequence identity with SEQ ID NO: 4. In some embodiments, the RBM25 or functional variant thereof comprises an amino acid sequence of SEQ ID NO: 4.

In some embodiments, the RBM25 polypeptide is a fragment that lacks the PWI domain. The PWI domain is found for example at amino acids of 750-843 of uniprot P49756-1.

In some embodiments, the RBM25 polypeptide is a fragment that lacks the C-terminal domain. The C-terminal domain is found for example at amino acids 610-843 of uniprot P49756-1. For example, the RMB25 polypeptide comprises at least amino acids 1-609 or at least 1-610 of SEQ ID NO: 4 or a sequence with at least 90%, or at least 95% sequence identity thereto. In some embodiments, the RBM25 polypeptide comprises at least 1-650, 1-700 or 1-749 of SEQ ID NO: 4 or a sequence with at least 90%, or at least 95% sequence identity thereto.

The splicing factor can be linked to the N-terminus or the C-terminus of dCas13d.

In some embodiments, the RBM25 polypeptide is fused to the N-terminus of dCasRx. In some embodiments, the RBM25 polypeptide is fused to the C-terminus of dCasRx.

### Linkers

In some embodiments, the fusion protein of the present disclosure further comprises a linker. A linker may be incorporated to provide appropriate distance and/or spatial orientation of the dCas13, optionally dCasRx, and the splicing factor. Any suitable linker may be used with the fusion protein.

For example variants of Gx4S and XTEN linkers have been used with the constructs described and provided similar results.

In some embodiments, the fusion protein of the present disclosure further comprises a linker. In some embodiments, the linker is a glycine-serine linker.

In some embodiments, the linker comprises an amino acid sequence of at least 90%, or at least 95% sequence identity with SEQ ID NO: 7. In some embodiments, the linker comprises an amino acid sequence of at least 90% sequence identity with SEQ ID NO: 7. In some embodiments, the linker comprises an amino acid sequence of at least 95% sequence identity with SEQ ID NO: 7. In some embodiments, the linker comprises an amino acid sequence of SEQ ID NO: 7.

The fusion protein disclosed herein can comprise other motifs, such as a tag, optionally a HA tag or a fluorescent tag.

In some embodiments, the dCasRx-RBM25 polypeptide fusion protein comprises an amino acid sequence of at least 90%, or at least 95% sequence identity with SEQ ID NO: 1. In some embodiments, the fusion protein comprises an amino acid sequence of at least 90% sequence identity with SEQ ID NO: 1 In some embodiments, the fusion protein comprises an amino acid sequence of at least 95% sequence identity with SEQ ID NO: 1. In some embodiments, the fusion protein comprises an amino acid sequence of SEQ ID NO: 1.

### III. Nucleic Acids, Cells, Compositions, and Kits

In another aspect, provided herein is a nucleic acid comprising a polynucleotide sequence encoding a fusion protein of the present disclosure.

The nucleic acid can for example be in a vector. Accordingly also provides is a vector comprising the nucleic acid.

The term “vector” as used herein refers to a molecule used as a vehicle to introduce foreign nucleic acids into a cell. A vector can, for example, be a plasmid, a phage, a cosmid, a transposon, or a viral vector, such as an adeno-associated viral vector (AAV), an adenoviral vector, a retroviral vector, a transposon vector or a lentiviral vector. The transposon vector can for example be a piggyBAC vector.

The vector can be used to introduce a nucleic acid encoding the fusion protein into a cell for example, in vitro, or ex vivo. The vector may be introduced into the cell by any suitable method known in the art. Suitable methods include but are not limited to transfection, transduction, infection, electroporation, sonoporation, nucleofection, and microinjection.

The vector can be used for transient expression of the fusion protein of the present disclosure, or for stable integration of the nucleic acid encoding the fusion protein into the genome of the host cell.

The vector can comprise one or more other expression cassettes, which can for example be separated by a IRES or P2A sequence.

The nucleic acid sequence encoding the fusion protein can be operably linked to one or more regulatory elements, such as a promoter.

Examples of such regulatory elements include: a transcriptional promoter and enhancer or RNA polymerase binding sequence, and/or a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the vector. Suitable regulatory elements may be derived from a variety of sources, including bacterial, fungal, viral, mammalian, or insect genes.

The term “promoter” or “promoter sequence” generally refers to a regulatory DNA sequence capable of being bound by an RNA polymerase to initiate transcription of a downstream (i.e. 3′) sequence to generate an RNA. Suitable promoters may be derived from any organism and may be bound or recognized by any RNA polymerase. Suitable promoters include for example strong constitutive promoters such as EF1A and CAG promoters.

Also provided in another aspect is a cell, optionally an isolated and/or a stable recombinant cell, expressing the fusion protein and/or comprising the nucleic acid described herein.

Stable recombinant cells can be made by for example being transformed, transfected or transduced with a vector comprising a nucleic acid, optionally any nucleic acid described herein. In some embodiments, the recombinant cells expressing the fusion protein are made using HEK293T, HEK293S, HEK293F and/or CHO cells, and said recombinant cells may be used to produce recombinant polypeptides and/or fusion proteins, for example under conditions suitable for in vivo use.

In some embodiments, the cells used to make recombinant cells expressing the fusion protein also express one or more guide RNAs that targets a pre-mRNA.

A further aspect is a composition comprising the fusion protein, the isolated nucleic acid, the vector or the recombinant cell described herein.

The composition can also comprise a suitable diluent or carrier. In some embodiments, the carrier is a pharmaceutically acceptable carrier.

In some embodiments, the composition is a pharmaceutical composition comprising the fusion protein and a pharmaceutically acceptable carrier.

Yet another aspect provided herein is a kit comprising the fusion protein, the isolated nucleic acid, the vector, the composition, or the recombinant cell described herein.

In some embodiments, the kit further comprises a nucleic acid that can express one or more guide RNAs. For example, the one or more guide RNAs can be one guide RNA, two guide RNAs or three guide RNAs. The one or more guide RNAs can also be one or two guide RNAs, optionally per target.

In some embodiments, the one or more guide RNAs targets an intron downstream or upstream of an exon of a pre-mRNA.

In some embodiments, the one or more guide RNAs targets an exon.

In some embodiments, the kit further comprises one or more guide RNAs, optionally wherein the guide RNAs are synthetic RNAs. The synthetic RNAs can be modified, for example, to comprise a modified backbone and/or one or more modified nucleotides. Modification can improve stability of the RNA and/or specificity of the guide RNA (see e.g. Ryan, Daniel E et al. Nucleic acids research vol. 46,2 (2018): 792-803, the content of which is incorporated herein by reference in its entirety).

The kit can in some embodiments comprising a fusion protein described herein or nucleic acid encoding said fusion protein along with one or more guide RNAs, optionally synthetic guide RNAs, or a nucleic acid that can express one or more guide RNAs.

### IV. Methods

It is demonstrated herein that when a cell expresses the fusion protein of the present disclosure and a gRNA that targets an intron downstream of an exon, a higher frequency of inclusion of the exon is observed as compared to dCas13d alone or dCas13d fused to the splicing factor RBFOX1. It is also demonstrated herein that expressing just one guide RNA that targets the intron can result in efficient activation of the exon, resulting in a higher frequency of inclusion of the exon.

The present disclosure further demonstrates that dCasRx-RBM25 is a potent activator of diverse endogenous alternative exons, promoting as demonstrated below, the inclusion of 88% (14/16) tested exons by more than 20% when tethered to proximal downstream intronic sequences. Like dCasRx, dCasRx-RBM25 also potently inhibited splicing when tethered to exonic sequences, an observation that was leveraged to simultaneously activate and repress pairs of endogenous exons through multi-targeting gRNA arrays. Finally, dCasRx-RBM25 affords a remarkable degree of on-target specificity for modulation of target exons.

Hence it is demonstrated herein that the fusion protein can be used to activate a first exon and repress a second exon by co-expressing with a guide RNA that targets an intron downstream of the first exon another guide RNA that targets the second exon in the same cells.

It is also submitted, that the fusion proteins of the present application such as dCasRx-RBM25, offers one or more key advantages over other methods used to control splicing. First, its components can all be genetically encoded in expression vectors stably integrated into genomes, offering an advantage over ASO delivery, which is transient in nature. Second, compared to CRISPR-nuclease directed exon-skipping approaches, such as exonic deletion using CHyMErA¹² or pgFarm¹³, or splice-site base editing methods^{41,42,} targeting with dCasRx-RBM25 for example, is not constrained by PAM requirements. Instead, it requires only a single gRNA to target each exon, and it further affords multiplexed targeting without loss of exon skipping efficiency. Moreover, because dCasRx-RBM25 acts at the level of RNA, it avoids undesirable changes to the genome such as genotoxic double-strand DNA breaks. Third, in contrast to some other reported methods for controlling target splicing events (refer to Introduction), dCasRx-RBM25 results in the specific modulation of splice isoform ratios without impacting the expression levels of the targeted transcript.

As demonstrated by RNA-seq profiling, both the activation and repression of endogenous exons by dCasRx-RBM25 are highly specific. Guide RNAs directing dCasRx minimally require 18 nt of complementarity for efficient target recognition, and dCasRx lacks any bystander cleavage activity, likely explaining its high specificity 20,43. In contrast, engineered RNA-binding domains employing Pumilio proteins are limited to binding 8-nt RNA targets15, which can occur over 50,000 times by chance in the human genome, compared to 0.05 times for an 18-nt RNA target sequence. Thus, targeting specificity represents a major advantage of dCasRx-RBM25 over methods that inherently recognize shorter RNA sequences.

Accordingly, the present disclosure identifies dCasRx-RBM25 as a powerful tool for the individual and combinatorial modulation of endogenous alternative exons.

Accordingly, in another aspect, provided herein is a method of modulating splicing in a cell, comprising introducing into the cell the fusion protein of the present disclosure, and one or more guide RNAs, wherein the one or more guide RNAs targets a first intron downstream of a first exon of a first pre-mRNA.

In some embodiments, targeting an intron downstream of an exon results in activation of the exon and inclusion of the exon in the mature mRNA.

In some embodiments, modulating splicing results in activation of an exon.

As used herein, the terms “activating an exon”, “exon activation” and the like mean inclusion of an alternative exon during processing of a pre-mRNA so that the mature mRNA comprises the exon.

As used herein, the terms “repressing an exon”, “exon repression” and the like mean exclusion of an alternative exon during processing of a pre-mRNA so that the mature mRNA does not comprise the exon.

As used herein, the term “modulating splicing” or the like means changing alternative splicing events, resulting in a change in the frequency of different mature mRNA isoforms. For example, modulating splicing can mean an increase in the inclusion of an exon of a target pre-mRNA, resulting in a higher frequency of the isoform that includes the exon. Modulating splicing can mean changing alternative splicing of more than one target pre-mRNA.

As used herein, the term “guide RNA” or “gRNA” means an RNA that targets the Cas to a sequence, for example, of a pre-mRNA. For example, the guide RNA can target the fusion protein of the present disclosure to a pre-mRNA. The guide RNA can comprise a spacer sequence and a direct repeat.

Various algorithms can be used to design guide RNAs and are known in the art. For example, Cas13design (https://cas13design.nygenome.org/) is a tool to design Cas13d guide RNAs.

In some embodiments, the one or more guide RNAs or the one or more additional guide RNAs is one guide RNA. In some embodiments, the one or more guide RNAs or the one or more additional guide RNAs is two guide RNAs. In some embodiments, the one or more guide RNAs or the one or more additional guide RNAs is three guide RNAs. In some embodiments, the one or more guide RNAs or the one or more additional guide RNAs is four guide RNAs. In some embodiments, the one or more guide RNAs or the one or more additional guide RNAs is five or more guide RNAs. For example, the fusion proteins, methods, nucleic acids, cells, compositions and/or kits and other products or assays described herein can comprise one or more guide RNAs. The one or more guide RNAs can be one guide RNA, two guide RNAs or three guide RNAs. The one or more guide RNAs can also be one or two guide RNAs. For example, the number of guide RNAs can be per target site e.g., per exon or intron targeted.

The one or more guide RNAs can be single guide RNAs, or they can be an array of guide RNAs. For example, the array of guide RNAs can comprise two or more spacer sequences, separated by direct repeat sequences. Some Cas nucleases, such as Cas13d, possess nuclease activity that allows self-processing of the array into mature guide RNAs.

In some embodiments, the one or more guide RNAs is provided as an array.

The method can further comprise introducing into the cell one or more additional guide RNAs targeting a second intron downstream of a second exon of the first pre-mRNA.

The method can further comprise introducing into the cell one or more additional guide RNAs targeting a second intron downstream of a second exon of a second pre-mRNA.

The method can further comprise introducing into the cell one or more additional guide RNAs targeting a second exon of the first pre-mRNA.

The method can further comprise introducing into the cell one or more additional guide RNAs targeting a second exon of a second pre-mRNA.

In some embodiments, targeting an exon results in suppression of the exon.

The one or more additional guide RNAs and/or the one or more guide RNAs can be provided as one or more arrays of guide RNAs.

In some embodiments, the method further comprises introducing into the cell one or more further additional guide RNAs.

The further additional guide RNAs can target other introns and/or other exons of the same pre-mRNAs or different pre-mRNAs.

The one or more further additional guide RNAs, one or more additional guide RNAs and/or the one or more guide RNAs can be provided as one or more arrays of guide RNAs.

The method disclosed herein can be used to modulate expression of a protein, wherein the protein is translated from a mature mRNA processed from the pre-mRNA targeted by the one or more guide RNAs.

Any suitable methods to introduce the fusion protein and the guide RNAs into the cell are contemplated.

For example, one or more expression vectors comprising sequences encoding the fusion protein and/or the guide RNAs can be used. Both transient expression and stable integration into the genome are contemplated.

The fusion protein and/or the guide RNAs can be introduced into the cell directly by methods such as electroporation and nanoparticles. Nanoparticles that can be used for delivery include for example lipid-based nanoparticles, polymer-based nanoparticles, and cell-derived nanoparticles such as exosomes (see e.g. Kong H et al. Advanced Science. 2021 December; 8(24):2102051, the content of which is incorporated herein by reference in its entirety). When delivered directly, the guide RNA can be a synthetic RNA. Such synthetic RNAs can be chemically modified to for example improve stability. The guide RNA and the Cas can be complexed to form a ribonucleoprotein for delivery (see e.g. Rathbone et al. The CRISPR Journal 5.3 (2022): 397-409, the content of which is incorporated herein by reference in its entirety).

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Methods

### Cell Culture

HEK293T and Neuro-2A (N2A) Flp-In cells25 were maintained in Dulbecco's Modified Eagle's Medium (DMEM) supplemented with 10% fetal bovine serum (FBS), glutamine, sodium pyruvate, MEM non-essential amino acids, and penicillin/streptomycin. All cell lines were routinely tested for mycoplasma contamination.

### Cloning and Plasmids

Expression plasmids for RBFOX1N-dCasRx-C (Addgene #118635), and gRNAs targeting the SMN2 downstream intron (Addgene #118646-118648) are as previously described²¹. For Mef2d reporter experiments, gRNAs were cloned into the pXR003 backbone (Addgene #109053) by annealed oligonucleotide ligation.

For endogenous targeting, we re-cloned the coding sequences of dCasRx-RBM25 and RBFOX1N-dCasRx-C into the pXR002 dCasRx expression backbone (Addgene #109050). We also generated a custom gRNA expression plasmid containing an enhanced CasRx processed direct repeat36, together with a SV40-driven BFP expression cassette to monitor transfection efficiency.

The Mef2d reporter is as previously described27. The SMN2 reporter was generated through Gibson assembly, using the pCI-SMN2 minigene26 as a template for the SMN2 exon and its surrounding sequences.

### Generation of the dCasRx-SF Library

A Gateway destination vector with an N-terminal dCasRx coding sequence with NLS and HA sequences was first constructed, followed by a glycine-serine-linker and attR-ccdB-attR. Selected splicing factor ORFs were individually picked from the human ORFeome collection in pDONR221/223 format and LR cloned to generate dCasRx-SF constructs. Clones were verified by restriction digest.

### Transient Transfection of Cell Lines

For initial reporter experiments and individual validations, 12,500 N2A Flp-In or 20,000 HEK293T cells were seeded in 96-well plates the day before transfection. dCasRx, gRNA, and reporter plasmids were co-transfected at a ratio of 50 ng:50 ng:3 ng for N2A Flp-Ins, or 75 ng:75 ng:5 ng for HEK293Ts, using a 3:1 ratio of Lipofectamine 3000 to DNA. In the case of multiple gRNAs, the amount of each gRNA plasmid was divided evenly, keeping constant the total gRNA amount. For the Mef2d reporter, doxycycline was added 24 h post-transfection to N2A Flp-In cells at a final concentration of 1 ug/mL to induce reporter expression. Cells were harvested 48 hours post-transfection for luciferase assays or RNA extraction.

To target endogenous alternative exons, 25,000-30,000 HEK293T cells were seeded in 96 well plates the day before to reach a confluency of ˜80% at transfection. 200 ng of dCasRx, RBFOX1N-dCasRx-C, or dCasRx-RBM25 expression vector was co-transfected with 200 ng gRNA expression vector using X-tremeGENE 9 (Roche) at a 3:1 ratio to DNA. RNA was harvested 48 hours post-transfection.

For protein expression analysis, 250,000 HEK293T cells were seeded the day before in 12-well plates. 1ug of dCasRx or dCasRx-SF expressing plasmids were transfected using Lipofectamine 3000. Cells were harvested after 48 hours for Western blotting.

### Dual-Luciferase Splicing Reporter Screens

For high-throughput reporter screens, 12,500 N2A Flp-In cells or 20,000 HEK293T cells were seeded in 96-well plates the day before transfection. For each well, 50 ng of dCasRx or dCasRx-SF plasmid was co-transfected with 50 ng gRNA expression vector and 3 ng of splicing reporter using polyethylenimine (PEI) at a 4:1 ratio to DNA. For the Mef2d reporter, doxycycline was added 24 h post-transfection to N2A Flp-In cells at a final concentration of 1 ug/mL to induce reporter expression. 48 hours post transfection, cells were washed with PBS and lysed in 80 uL HENG buffer (20 mM HEPES-KOH (pH 7.9), 150 mM NaCl, 2 mM EDTA, 5% glycerol, 0.5% Triton X-100, supplemented with protease inhibitors) with shaking for 15 minutes at room temperature. To perform dual-luciferase assays, 20 uL of cell lysate was transferred to 384-well plates and first mixed with 20 uL firefly luciferase buffer (150 mM Tris-HCl (pH 8), 75 mM NaCl, 3 mM MgCl2, 0.25% Triton X-100, with 15 mM DTT, 0.6 mM Coenzyme-A, 0.45 mM ATP, and 250 ug/mL D-luciferin added immediately before assay) and incubated at room temperature with shaking for 10 minutes. Firefly luminescence was measured with a plate reader (Biotek Synergy 5, Biotek) with 100 ms integration time per well. Next, 20 uL of Stop & Glo buffer was dispensed to each well (20 mM Tris-HCl (pH 7.5), 150 mM KCl, 45 mM EDTA (pH 8), 0.5% Tergitol NP-9, with 60 uM PTC124, 50 mM thioacetamide, and 10 ug/mL furimazine added immediately before assay) and the plates were incubated at room temperature with shaking for 5 minutes. Nanoluc luminescence was then measured using the same settings. Complete quenching of the firefly signal was verified by control wells transfected with a firefly luciferase expression plasmid (data not shown). All washing and dispensing steps were performed using an automated plate filler/washer (Biotek), and transfections were performed using a 96-channel electronic pipette (VIAFLO 96, Integra Biosciences).

### RNA Extraction, RT-PCR, and RT-qPCR Assays

Total RNA was extracted using the QIAGEN RNeasy Mini Kit with cells directly lysed in RLT buffer supplemented with 1% beta-mercaptoethanol. To assay exon inclusion, RT-PCR assays were performed using the QIAGEN OneStep RT-PCR kit with 40 ng of total RNA per 10 μl reaction, using primers annealing to the flanking constitutive exons of the alternative exon. Reaction products were separated on 2-3% agarose gels, and percent spliced in (PSI) was calculated as the intensity of the exon-inclusion band divided by the sum of the exon-inclusion and exon-skipped band intensities. For RT-qPCR, first-strand cDNAs were synthesized from 250 ng of total RNA using the Thermo Scientific Maxima H Minus First Strand cDNA synthesis Kit, as per the manufacturer's instructions, and then diluted 1/20. qPCR reactions were performed in a volume of 10 μl with 4 μl of diluted cDNA using the SensiFAST SYBR No-ROX Kit following manufacturer's instructions. Expression fold change was calculated using the ddCT method, normalized to the housekeeping gene GAPDH. Primer sequences used for RT-PCR and RT-qPCR reactions are available upon request.

### Western Blotting

Cells were lysed in RIPA buffer (50 mM Tris-HCl (pH 7.4), 150 mM NaCl, 1% NP-40, 0.1% SDS, 0.5% sodium deoxycholate) containing protease inhibitors, and sonicated for 20 seconds. Lysates were centrifuged at 15,000 g for 15 minutes, and the supernatant was mixed with SDS loading buffer and boiled for 5 minutes. Proteins were separated by SDS-PAGE on 8% Tris-Glycine gels, wet transferred onto a PVDF membrane overnight at 30V, and blocked with 5% milk in Phosphate-Buffered Saline with 0.1% Tween-20 (PBST). Membranes were incubated at 4° C. overnight with primary antibodies: anti-HA (Roche 3F10), anti-alpha-tubulin (Sigma T6074). Membranes were washed 3 times with PBST and incubated with secondary antibodies for 1 hour at room temperature. Membranes were then washed 5 times with PBST, incubated with ECL (Perkin-Elmer), and imaged by chemiluminescence (LI-COR Odyssey imager).

### RNA-Sequencing

Cells were transfected as described above and RNA extracted 48 hours post-transfection. 500 ng of total RNA was used for generation of polyA+ libraries with the NEB Next Ultra II Directional kit. Sequencing was performed by the Donnelly Sequencing Centre using a Nova-Seq 6000 instrument and S1 flow cell to an average depth of 50 million paired-end 150 nt reads.

Alternative Splicing analysis. Raw reads were aligned using the vast-tools pipeline, version 2.5.1 (https://github.com/vastgroup/vast-tools), to the hg38 VastDB library. Exons were filtered for coverage and junction balance, requiring a vast-tools quality score of SOK/OK/LOW and a balance score of OK/B1/B2/BI/Bn in all samples. Exons were considered significantly changing if the absolute change in PSI was greater than 15 and the expected minimum change (MV) was greater or equal to 0.1 at p>0.95 according to vast-tools diff module.

Gene Expression analysis. Raw reads were aligned to the hg38 genome using STAR v.2.7.10, and read counts per gene were computed us-g—quantMode GeneCounts, with the Ensembl hg38 GTF annotations (release 109). edgeR was used to calculate RPKM values, normalize counts using the TMM method, and calculate differential expression for all protein-coding genes using the quasi-likelihood F test. Genes were considered differentially expressed if they showed greater than two-fold change (log2fc>1) at FDR<0.05 and were expressed at a minimum of 2 RPKM in at least one condition.

### Example 2

### Assaying dCasRx-Splicing Factors Using Dual-Luciferase Splicing Reporters

Since many alternative exons show tissue-specific inclusion and are regulated by different sets of endogenous splicing factors, it is possible that the sequence context of an exon affects the degree to which it responds to the recruitment of specific dCasRx-splicing factor fusions (dCasRx-SFs). An optimal dCasRx-SF should, however, efficiently activate splicing independently of sequence and cellular context, and ideally also in different species such as human and mouse. Therefore, two different alternative exons were chosen—the murine Mef2d microexon and human SMN2 exon 7—for screening of dCasRx-SFs. The Mef2d microexon belongs to a class of short (3-27 nt) alternative exons that is spliced in the nervous system dependent on Srrm4, a neural-specific splicing activator, as well as additional factors^{24,25}. In comparison, SMN2 exon 7 is a longer (54 nt) alternative exon that, due to a splicing enhancer-disrupting mutation, has ubiquitously low basal inclusion levels compared to a paralogous exon in the SMN1 gene, which is disrupted by mutations in spinal muscular atrophy²⁶. These alternative exons thus allow the testing of dCasRx-SFs for both context-dependent and-independent splicing control.

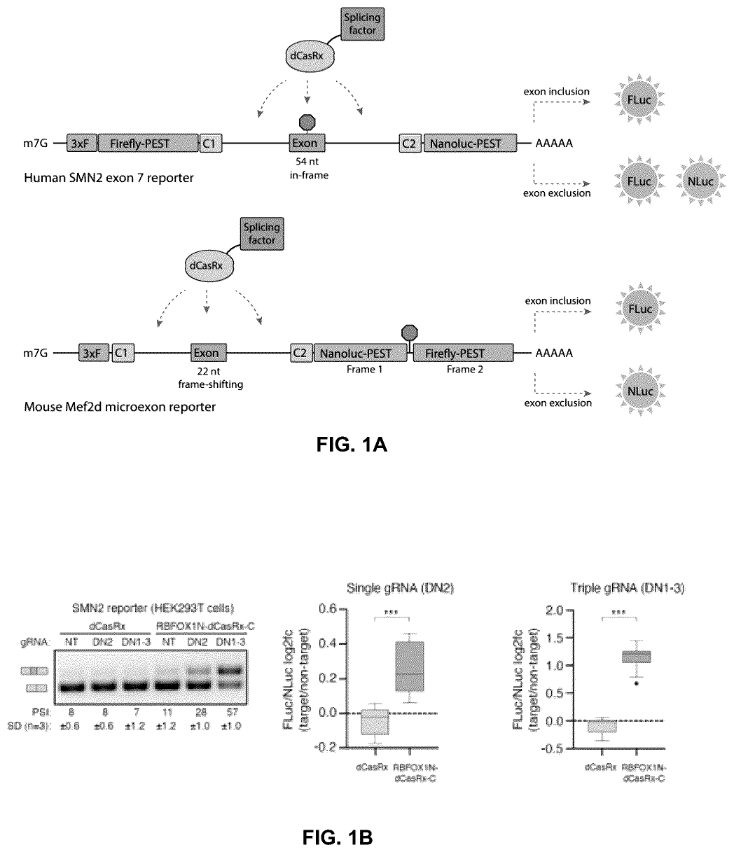

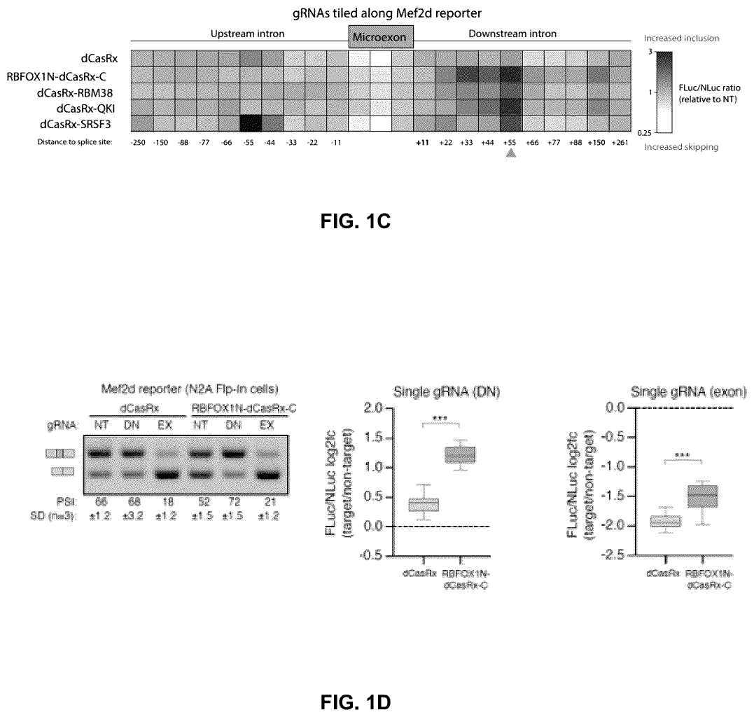

To develop a high-throughput recruitment assay for dCasRx-SFs, dual-luciferase splicing reporters for these two exons were employed (FIG. 1A). For the Mef2d reporter²⁷, a single-nucleotide insertion within the microexon results in a reading frame shift, such that firefly luciferase (FLuc) is expressed upon exon inclusion, and Nanoluciferase (NLuc) is expressed upon skipping. For the SMN2 reporter, due to the native stop codon within exon 7, the reporter expresses FLuc upon inclusion, and expresses both FLuc and NLuc upon exon skipping (FIG. 1A). Both luciferases were tagged with PEST degradation sequences to facilitate their rapid turnover, thereby providing greater sensitivity of detected splicing changes in response to expression of dCasRx-SFs. Thus, quantification of the relative ratios of the luciferase signals affords a rapid readout for changes in splice isoform levels and is suitable for screening in a multi-well format.

The reporters were tested for splicing activation by the RBFOX1N-dCasRx-C construct²¹. The SMN2 reporter was co-transfected into HEK293T cells with RBFOX1N-dCasRx-C or dCasRx constructs, together with three downstream intron-targeting gRNAs (DN1-3)²¹. Recapitulating previous results, moderate exon 7 activation (17% increase in percent spliced in [PSI]) using a single downstream-targeting gRNA (DN2) was observed, whereas co-transfection of all three downstream-targeting gRNAs (DN1-3) results in markedly higher activation (46% increase in PSI), specifically with RBFOX1N-dCasRx-C expression (FIG. 1B). To identify optimal gRNAs for the Mef2d reporter, a panel of 23 gRNAs tiled along the pre-mRNA were co-transfected with four dCasRx-SFs (including RBFOX1N-dCasRx-C) or dCasRx, and the reporter, into mouse neuroblastoma (N2A Flp-In) cells (FIG. 1C). This reveals that a gRNA positioned +55nt in the downstream intron (DN) results in a 20% increase in inclusion with RBFOX1N-dCasRx-C, whereas a gRNA that spans the exon (EX) leads to increased skipping (FIGS. 1C, 1D). Importantly, dCasRx alone does not result in detectable splicing activation when tethered downstream of either reporter but leads to efficient repression when tethered to the Mef2d exon. This demonstrates that while activation depends on the RBFOX1 domains of RBFOX1N-dCasRx-C, splicing repression can be achieved regardless of the fused protein.

Both luciferase readouts (FLuc and NLuc) were quantified and log2 fold-change (FC) values comparing luciferase ratios for tethered (targeting) versus untethered (non-targeting) conditions for both constructs were computed (FIGS. 1B, D). Reflecting RT-PCR-detected splicing changes, an increase in the relative luciferase ratios (log2FC>0) was observed when RBFOX1N-dCasRx-C is tethered downstream of both reporters, whereas tethering dCasRx alone results in minimal luciferase ratio changes that are also significantly lower than those of RBFOX1N-dCasRx-C (p=7.4×10\hat{ }−7, Wilcoxon test). Consistent with the RT-PCR data showing efficient splicing repression, there is a substantial decrease in luciferase ratios (log2FC<0) when dCasRx and RBFOX1N-dCasRx-C are tethered to the Mef2d exon (EX) (FIG. 1D). These results thus demonstrate that the SMN2 and Mef2d dual-luciferase splicing reporters afford reliable quantification of gRNA-targeted, dCasRx(-SF)-dependent alternative splicing changes and therefore are suitable for systematic screens of new dCasRx-SFs.

### Example 3

### Systematic Screening of dCasRx-Splicing Factors Reveals dCasRx-RBM25 as a Potent and Versatile Exon Activator

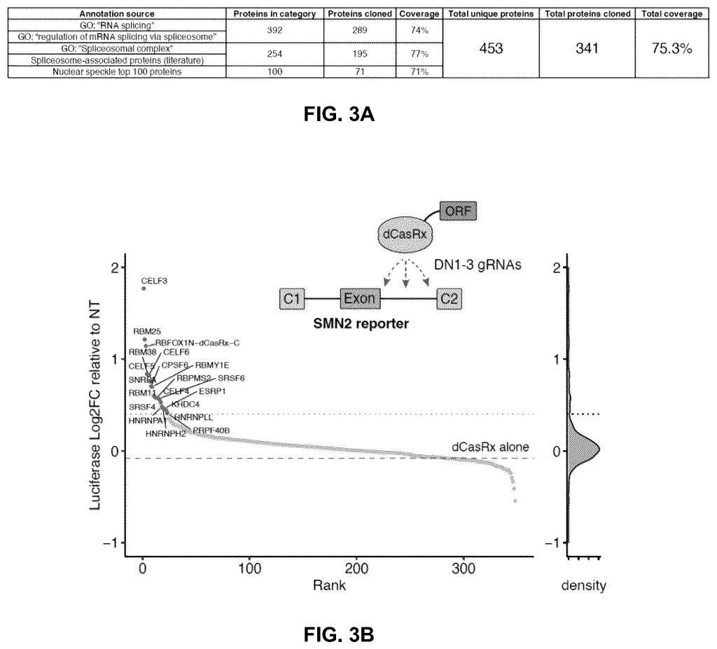

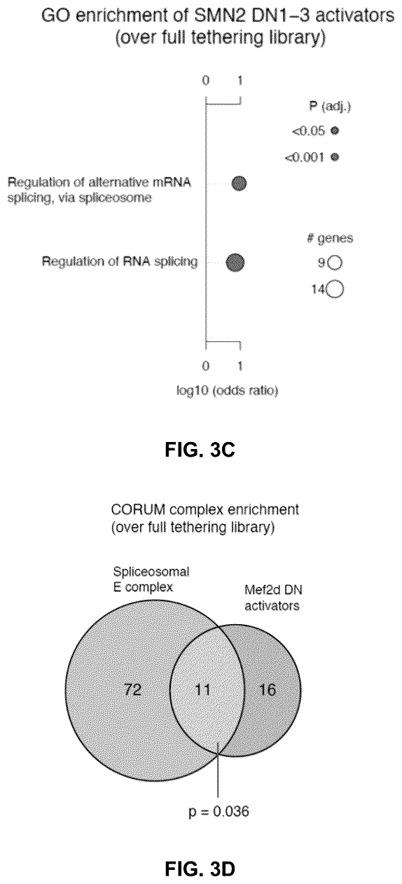

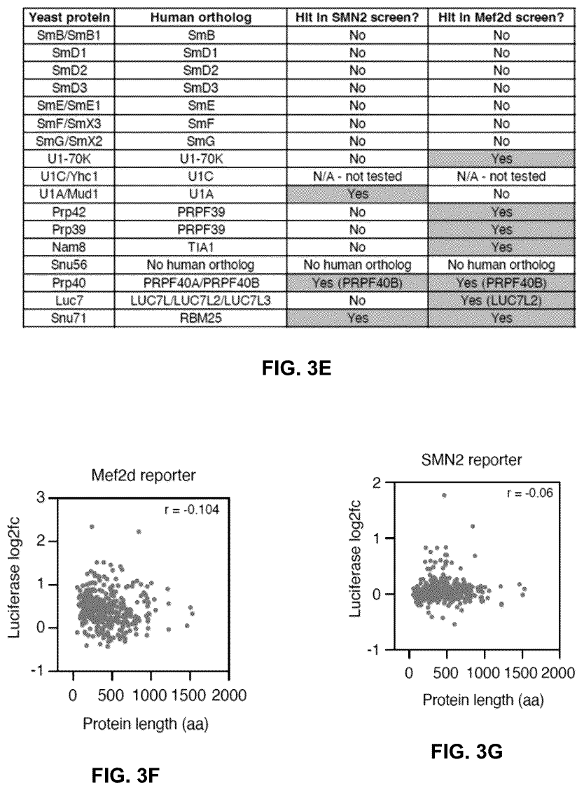

A comprehensive list of 453 unique human proteins with splicing-related functions was complied by merging annotations from Gene Ontology (GO), literature-curated spliceosomal proteins^{28,29}, as well as nuclear speckle proteins³⁰ (FIG. 3A). cDNAs corresponding to the longest available isoform for each protein from the human ORFeome collection³¹ were picked and fused to the C-terminus of an HA-tagged, NLS-dCasRx vector to generate a library of dCasRx-SF expression vectors. cDNAs for 341/453 (75%) of the target proteins were successfully cloned, resulting in a library comprising the large majority of known splicing factors in the human proteome (FIG. 3A, Table 1). Each dCasRx-SF expression vector was then co-transfected with the SMN2 reporter in HEK293T cells, or with the Mef2d reporter in N2A Flp-In cells, along with gRNA(s) targeting the downstream introns, or a non-targeting gRNA. The luciferase ratio of each tethered dCasRx-SF construct was normalized to its untethered (NT) control. Significant hits in each screen were determined by both luciferase log2FC and adjusted p-values from t-tests relative to the negative control (dCasRx) (Table 2).

Table 1. List of 341 splicing factors assayed in the study. For each splicing factor, column 2 indicates whether it is annotated with GO: RNA splicing or GO: regulation of mRNA splicing via spliceosome, column 3 indicates whether it is annotated with GO: spliceosomal complex or part of a list of literature-curated spliceosomal proteins^{28,29}, column 4 indicates whether it belongs to the top 100 nuclear speckle proteins as identified by TSA-MS³⁰. Column 5 indicates the source of the ORF clone—hORFEOME version 8.1, hORFEOME version 9.1, or a custom collection (OC). Column 6 lists its length (number of amino acids).
Table 2. Luciferase Log2FC scores for each dCasRx-SF construct and associated FDR values, when tethered to the SMN2 reporter with a single downstream gRNA (columns 2-3), SMN2 reporter with triple downstream gRNAs (columns 4-5), or the Mef2d reporter with a single downstream gRNA (columns 6-7).

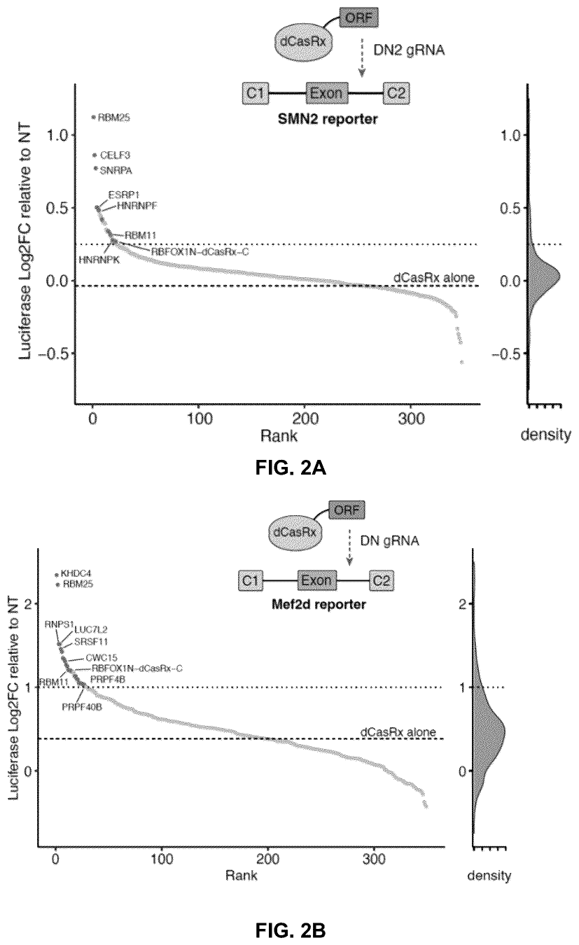

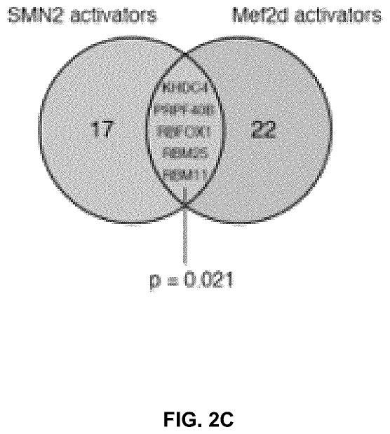

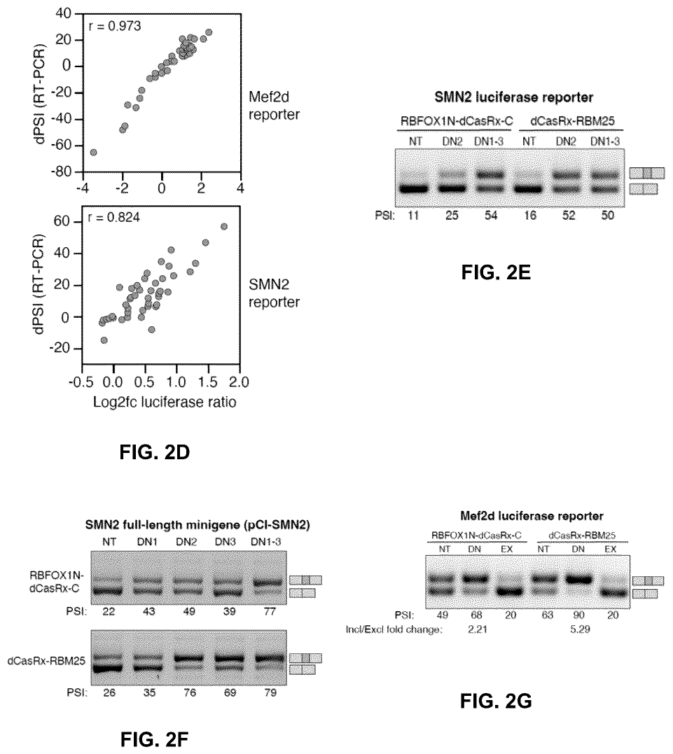

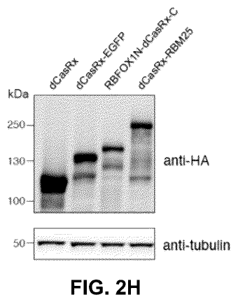

When tethered using a single gRNA downstream of SMN2 exon 7 (DN2), most dCasRx-SFs have a minimal effect on splicing, forming a normal distribution centered on dCasRx (FIG. 2A). Consistent with the results described above, RBFOX1N-dCasRx-C shows weak but significant activation. Importantly, we observe a pronounced ‘tail’ of ten stronger dCasRx-SF activators, with dCasRx-RBM25 showing the highest level of splicing activation. The same dCasRx-SF library was assayed with the SMN2 reporter following co-transfection of the three downstream gRNA constructs (DN1-3). Although most dCasRx-SF constructs still perform similarly to dCasRx and do not significantly activate exon 7 splicing, a more pronounced tail of activators is observed (FIG. 3B). In this screen, dCasRx-RBM25 ranks as the second strongest activator, behind dCasRx-CELF3.In parallel, the dCasRx-SF library was screened using the Mef2d microexon reporter in N2A Flp-In cells and a single downstream intronic gRNA (DN) (FIG. 2B). Like both SMN2 screens, most constructs show minimal effects, with a tail of activator constructs showing higher luciferase changes. In this context, RBFOX1N-dCasRx-C is the 14th-strongest activator, whereas dCasRx-RBM25 and dCasRx-KHDC4 are the two strongest activators, displaying a clear increase in strength over all other tested dCasRx-SFs.

At a false discovery rate (FDR) of <0.1, using a log2FC>0.4 cut-off for the human SMN2 triple-gRNA screen, and log2FC>1.0 cut-off for the mouse Mef2d screen, 22 and 27 significant activators were identified, respectively (FIG. 2C). Reflecting potential differences in exon, cell-type, and/or tether-position contexts, only five activators are shared between these screens: RBFOX1N-dCasRx-C, RBM25, RBM11, PRPF40B, and KHDC4 (p=0.021, hypergeometric test). RT-PCR validation assays of selected fusions reveal a high correlation between the luciferase readout and RT-PCR dPSI measurements for both reporters (SMN2: r=0.824, n=44; Mef2d: r=0.973, n=42) (FIG. 2D).

Whether the dCasRx-SF activators share related features was investigated. Notably, the length of the fusion protein does not correlate with the luciferase readout, ruling out non-specific steric effects as a confounding factor (FIGS. 3F-3G). GO and CORUM complex enrichment analysis of the hits versus the full tethering library reveals significant enrichment in terms related to the regulation of mRNA splicing for the SMN2 hits, and ‘spliceosome E complex’ (which forms at the earliest stage of spliceosome assembly) for the Mef2d hits (FIGS. 3C-3D). Interestingly, human homologs of all tested (7/7) yeast U1 snRNP-specific proteins^{32,33}, including RBM25 (a homolog of yeast SNU71), are activator hits for at least one reporter, whereas none (0/7) of the tested snRNP Sm proteins activate splicing (FIG. 3E). These findings suggest that tethering of dCasRx-SFs representing homologs of U1 snRNP proteins to proximal downstream intronic positions may function by stabilizing the binding of the core U1 snRNP particle to the 5′splice site.

dCasRx-RBM25 stands out as the strongest activator with consistent performance across both screens (FIGS. 2A-2B). Its luciferase-detected splicing changes at the RNA level in both reporters were validated, as well its activity using a previously described minigene (pCI-SMN2) comprising SMN2 exon 7, full length flanking introns and constitutive exons 6 and 826 (FIGS. 2E-2G). Remarkably, it requires only a single gRNA to activate SMN2 exon 7 to a similar level as RBFOX1N-dCasRx-C when all three gRNAs are expressed (FIGS. 2E, 2F). Moreover, western blotting using anti-HA antibody to detect dCasRx-fusions in transfected HEK293T cells reveals that the differences in activity between dCasRx-RBM25 and RBFOX1N-dCasRx-C are not due to different expression levels (FIG. 2H).

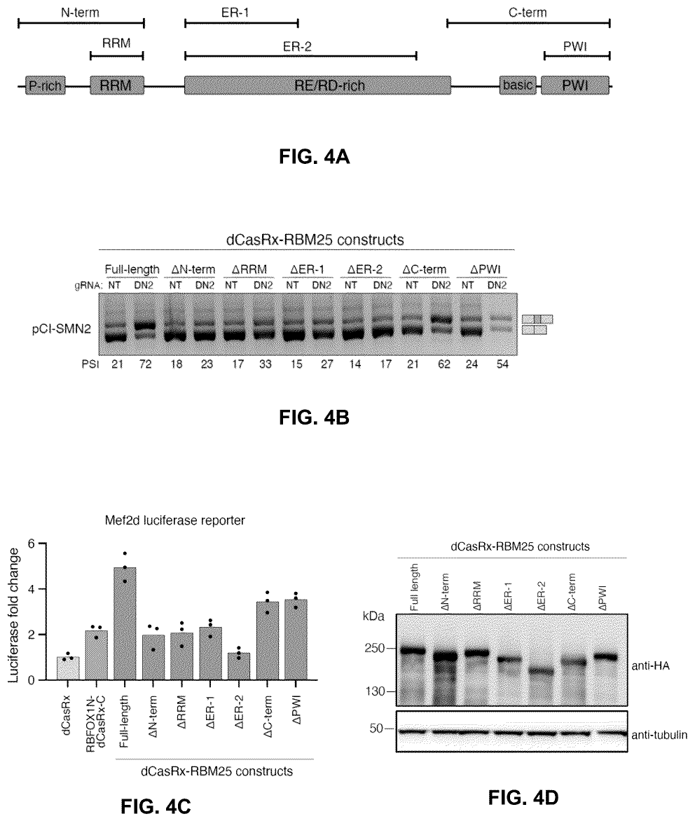

RBM25 is composed of an RNA Recognition Motif (RRM), arginine/glutamic acid/aspartate (RED)-rich domain, and nucleic acid binding PWI motif, interspersed with intrinsically disordered regions^{34,35}. Previous affinity purification coupled to mass spectrometry experiments showed that RBM25 interacts with a set of 41 splicing factors, including multiple hnRNPs, SR proteins, the U2 snRNP SF3 complex, and U1 snRNP components³⁵. Deletion of the RED and N-terminal regions resulted in abrogation of most interactions, whereas PWI deletion selectively affected interaction with the SF3 complex³⁵. To investigate which of these regions are responsible for the tethered splicing activity of dCasRx-RBM25, deletion derivatives of this fusion protein were assayed with single downstream gRNAs targeting the pCI-SMN2 and Mef2d luciferase reporter (FIGS. 4A-4C). Relative to full-length dCasRx-RBM25, deletion of each region, to varying extents, results in a loss of splicing activity for both reporters, with the N-terminus, RRM, and RED deletions drastically reducing activity, and the PWI deletion resulting in a minor reduction. Western blotting reveals that these effects are not due to differential expression of the dCasRx-RBM25 deletion derivatives (FIG. 4D). Collectively, these results suggest that the high degree of splicing activation observed for tethered dCasRx-RBM25 may be due to its interaction with numerous splicing factors involved in exon and intron definition at early stages of spliceosome assembly^{34,35}.

### Example 4

### Efficient Control of Endogenous Exons with dCasRx-RBM25

Having identified dCasRx-RBM25 as a strong activator of exon splicing using minigene reporters, whether it is also efficient at activating targeted endogenous alternative exons in human cells was investigated. A panel of 16 endogenous alternative exons in genes expressed in HEK293T cells was selected, including many with important biological functions and/or known disease associations (Table 3). This set includes the endogenous MEF2D microexon (which is skipped in HEK293T cells), as well as five exons (CD46, SPAG9, PLOD2, EVI5L, FLNB) that in previous experiments were targeted for repression, but not activation, by dCasRx²². For each exon, gRNAs targeting the downstream intron were designed using the Cas13design algorithm^{36,37}, and expressed from a vector carrying an enhanced CasRx direct repeat previously shown to increase knockdown efficiency³⁶. HEK293T cells were co-transfected with constructs expressing each gRNA and either dCasRx-RBM25, RBFOX1N-dCasRx-C, or dCasRx, and endogenous splicing changes were monitored by RT-PCR assays. Endogenous SMN2 exon 7 was not assayed since it is not possible to specifically monitor its inclusion due to endogenously expressed, constitutive SMN1 exon 7, which shares >99% sequence identity.

Table 3. List of 16 endogenous alternative exons chosen for targeted activation. Each alternative exon is listed with its length (column 2), its gene expression (RPKM) in HEK293T cells (column 3), and its biological relevance from literature searches (column 4).

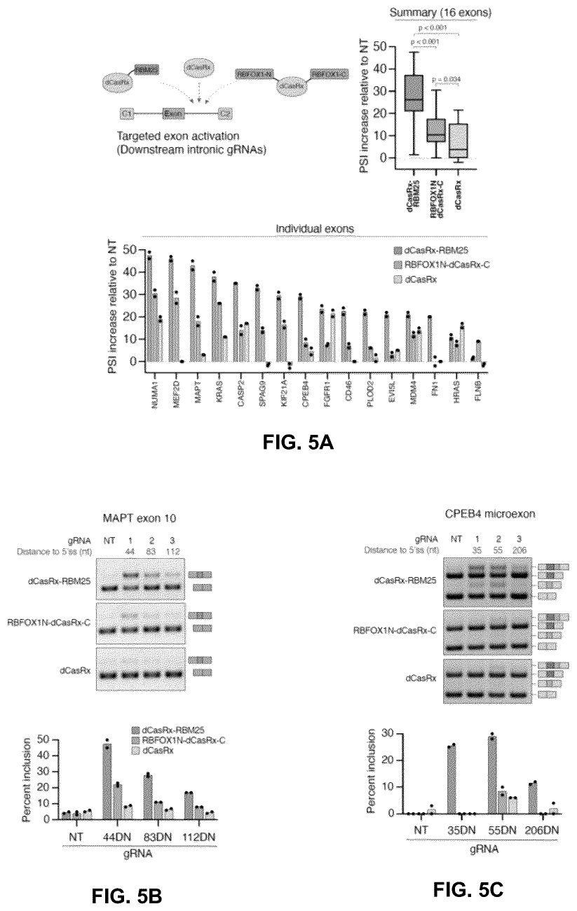

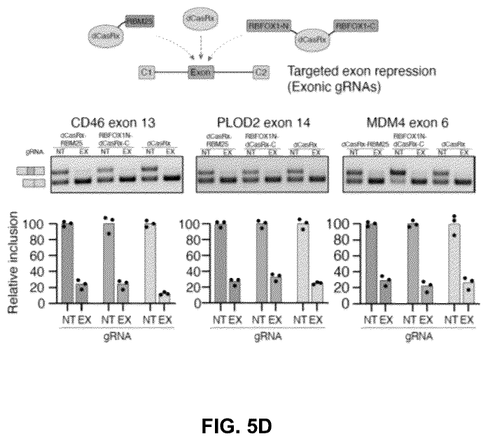

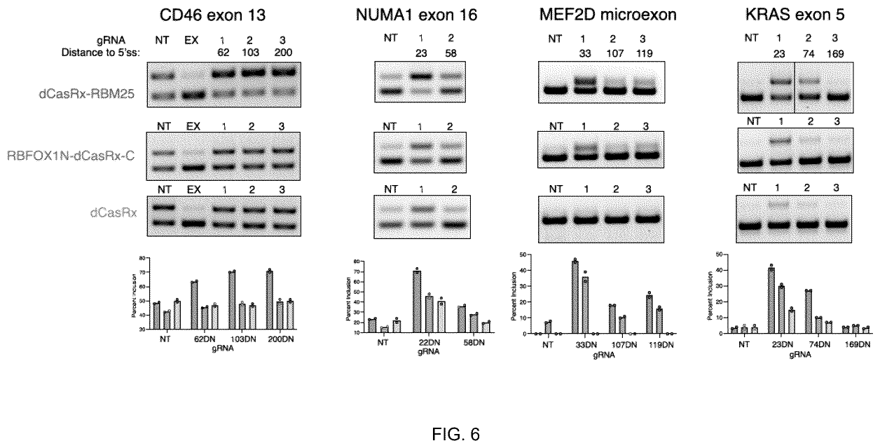

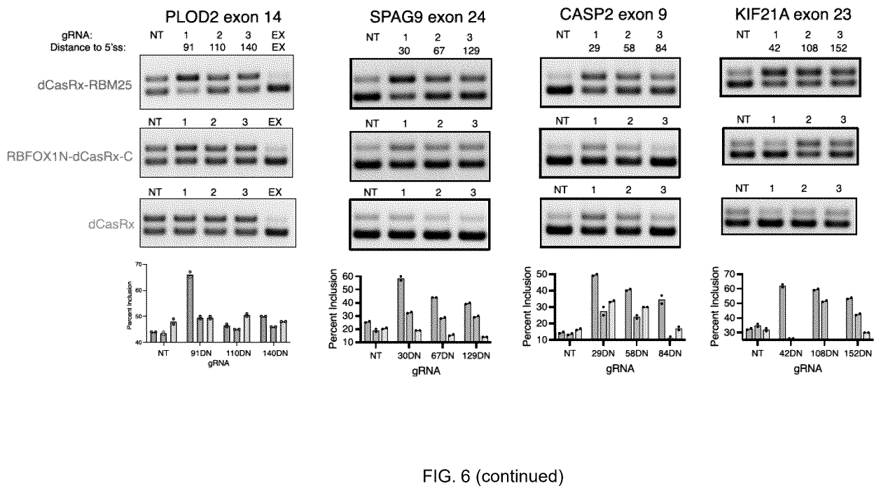

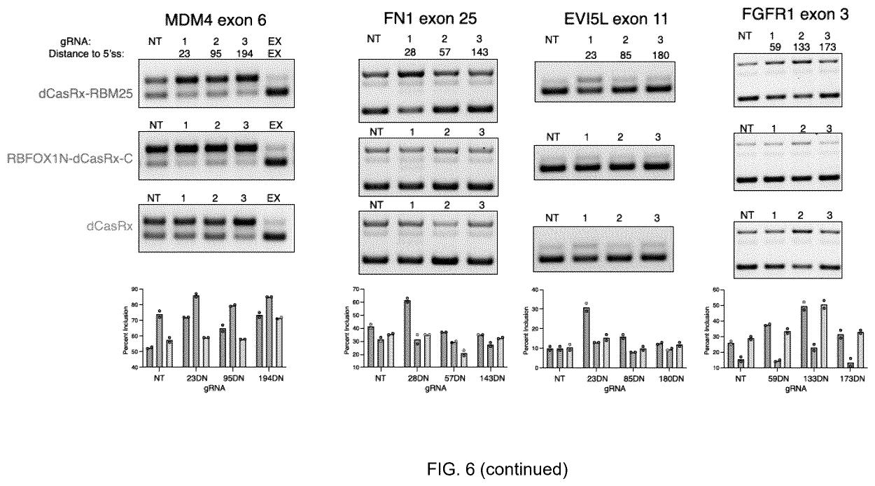

Remarkably, dCasRx-RBM25 outperforms RBFOX1N-dCasRx-C in the activation of 94% (15/16) of the tested alternative exons and achieves PSI increases of 20% or more for 14 of these exons with only a single downstream intron-targeting gRNA (FIG. 5A, FIG. 6). On average, dCasRx-RBM25 has more than twice the activation strength of RBFOX1N-dCasRx-C (median: 26.3% increase in PSI vs 10.5%; p<0.001, Wilcoxon test) (FIG. 5A). Among the successfully activated exons are MAPT exon 10 (FIG. 5B), whose altered splicing modulates Tau isoform balance and is associated with the progression of neurodegenerative diseases³⁸, and the CPEB4 neuronal microexon (FIG. 5C), the skipping of which causes autistic-like phenotypes in mice³⁹. In both cases, dCasRx-RBM25 led to higher activation across all gRNAs tested. In addition, we also targeted KIF21A exon 23, which was chosen for activation in a recent study describing a new RBFOX1-PUFc-based split artificial splicing factor (CREST)⁴⁰. Using the same gRNAs, higher KIF21A exon 23 activation by dCasRx-RBM25 was observed, especially when using the gRNA closest to the splice site (#1). Notably, while this gRNA did not activate splicing when expressed with RBFOX1N-dCasRx-C or CREST, a 30% PSI increase was achieved with dCasRx-RBM25 (FIG. 6).

For 6/8 tested exons with >10% activation by both dCasRx-RBM25 and RBFOX1N-dCasRx-C, the most effective gRNA is the same for both constructs, and is generally located within 20-70 nt of the 5′ splice site (e.g gRNA #1 for MAPT and gRNA #2 for CPEB4) (FIGS. 5B-5C, FIG. 6). As expected, tethering of dCasRx alone results in minimal changes (<10%) in inclusion levels for the majority (10/16) of tested exons. Interestingly, of the six exons for which tethering of dCasRx alone to the downstream intron results in >10% activation, tethering of dCasRx-RBM25 using the same gRNAs more than doubles their activation in 3/6 cases (KRAS, NUMA1, CASP2) (FIG. 5A, FIG. 6). As the effect of dCasRx alone likely results from blocking of intronic cis-elements, the further increase in PSI mediated by dCasRx-RBM25 suggests that it promotes splicing by simultaneously facilitating the recruitment of splicing components and sterically interfering with negatively acting elements in these cases.

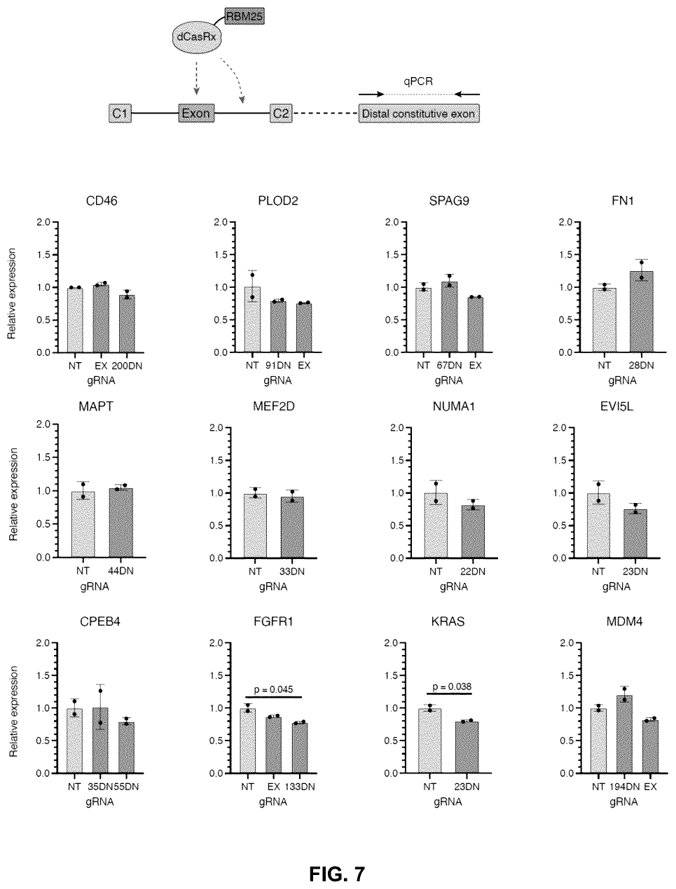

When tethered directly to exons, all three dCasRx(-SF) constructs achieve similarly high levels of splicing repression, with >70% relative decreases in inclusion compared to the respective non-targeting controls for CD46 exon 13, PLOD2 exon 14, and MDM4 exon 6 (FIG. 5D), of which CD46 exon 13 and PLOD2 exon 14 were previously successfully repressed by dCasRx alone²². This demonstrates that dCasRx-SF constructs retain the ability to effectively repress endogenous splicing when tethered directly to the exon, similar to what was observed with the Mef2d reporter (FIGS. 1D, 2G). Finally, RT-qPCR assays were performed to monitor distal constitutive exons within the same targeted genes and found minimal (<25%) differences in expression levels of these genes when comparing non-targeting, downstream intron-targeting, or exon-targeting dCasRx-RBM25, in all 12/12 tested genes (FIG. 7). This demonstrates that dCasRx-RBM25 specifically modulates isoform ratios without affecting overall transcript expression. Taken together, these results demonstrate that dCasRx-RBM25 is an efficient artificial splicing regulator that flexibly enables the targeted activation and repression of endogenous alternative exons in human cells.

### Example 5

### High On-Target Specificity of dCasRx-RBM25

Off-target effects are a source of concern with any genomic or transcriptomic engineering system. In the case of artificial splicing factors, two potential sources of off-targets are possible: (1) those mediated by overexpression of the fused splicing factor independent of gRNA targeting, and (2) those mediated by imperfect base pairing between the targeting gRNA and partially complementary RNA sequences. To assay both types of off-target effects, RNA sequencing (RNA-seq) was performed 48 h following co-transfection of expression constructs for dCasRx-RBM25, RBFOX1N-dCasRx-C, or dCasRx, with either non-targeting gRNAs, or a single gRNA targeting MAPT exon 10 for activation.

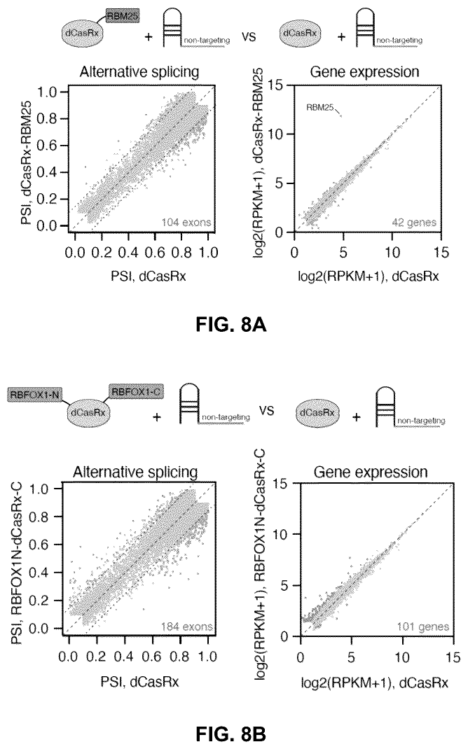

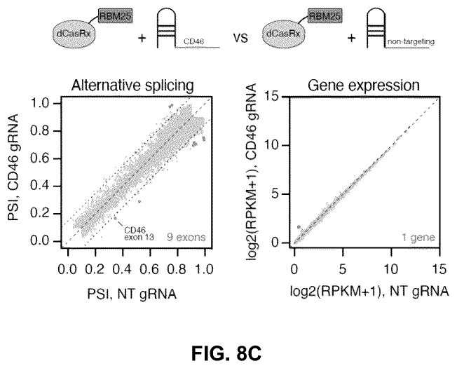

To characterize the extent of off-target effects potentially arising from overexpression of dCasRx-SFs without expression of targeting gRNAs, RNA-Seq analyses were performed to compare global alternative splicing and gene expression profiles following co-transfection of a non-targeting (NT) gRNA with either dCasRx-RBM25 or RBFOX1N-dCasRx-C, to co-transfection of dCasRx with the NT gRNA. In this experiment, 104 differentially spliced exons and 42 differentially expressed genes (DEGs) are detected with dCasRx-RBM25, whereas expression of RBFOX1N-dCasRx-C results in 184 differentially spliced exons and 101 DEGs (FIGS. 8A, 8B). Thus, despite its higher splicing activation efficiency, expression of dCasRx-RBM25 results in a smaller number of downstream effects compared to RBFOX1N-dCasRx-C. Transfected dCasRx is expressed at much higher levels compared to the dCasRx-SF constructs (FIG. 2H), potentially contributing to the number of detected changes.

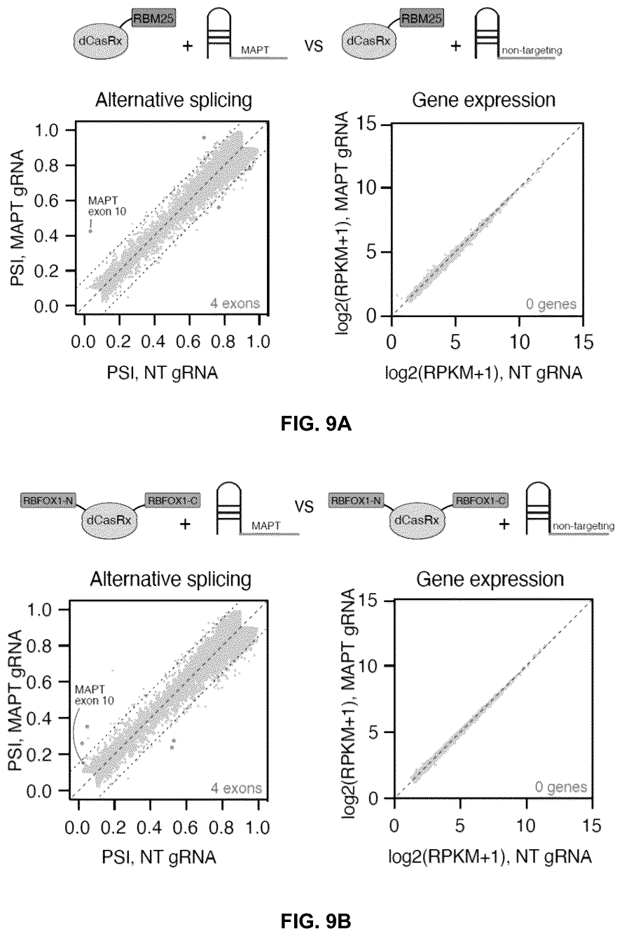

To assess gRNA-dependent off-target effects, global inclusion levels of annotated alternative exons between MAPT-targeting and non-targeting conditions for both dCasRx-RBM25 and RBFOX1N-dCasRx-C were compared. Strikingly, only four differentially spliced exons are detected in each case, out of a total of ˜11,000 detected alternative exons with sufficient read coverage (FIGS. 9A-9B). MAPT exon 10 is the most strongly changing exon in the transcriptome when targeted by dCasRx-RBM25 (dPSI=39.1, FIG. 9A). In contrast, it did not meet the significance cut-off when targeted by RBFOX1N-dCasRx-C (dPSI=12.8, FIG. 9B), due to the weaker activation efficiency of the RBFOX1 fusion (FIG. 5B). As another indication of on-target specificity, no DEGs were detected between MAPT-targeting and NT conditions, for both dCasRx-RBM25 and RBFOX1N-dCasRx-C. In addition, RNA-seq analyses were performed to investigate the specificity of dCasRx-RBM25 mediated repression of CD46 exon 13 using an exonic gRNA (FIG. 5D) and comparing it to the NT control. This reveals only eight differentially spliced exons in addition to the target exon, as well as only one DEG (FIG. 8C). Taken together, these RNA-seq analyses highlight the high degree of on-target specificity of dCasRx-RBM25 in mediating efficient exon activation and repression.

### Example 6

### Simultaneous Modulation of Endogenous Alternative Exons With gRNA Arrays

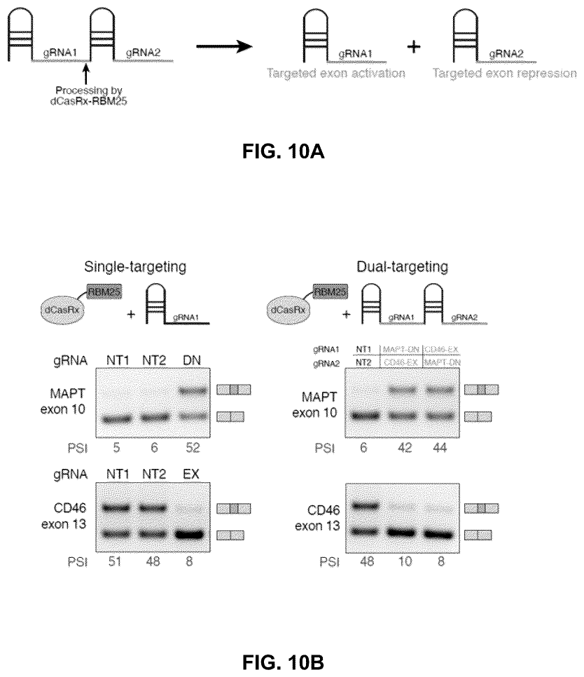

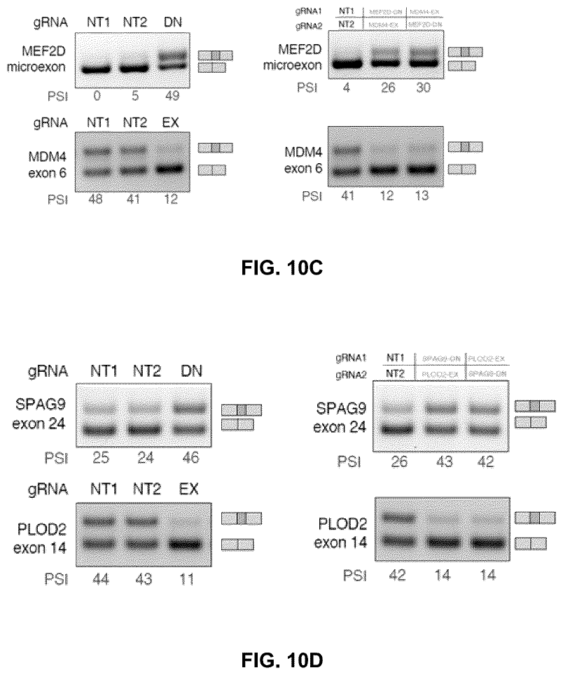

dCasRx retains its ability to process gRNA arrays into individual gRNAs, affording the potential of multiplexing exon targeting²¹. However, multiplexed targeting of endogenous exons has not been previously achieved. To test the ability of dCasRx-RBM25 to simultaneously activate and repress endogenous alternative exons, gRNA arrays with two guide spacers flanked by 36-nt enhanced CasRx direct repeats were generated, with one gRNA spacer targeting an exonic sequence for repression and the other gRNA spacer targeting the downstream intron of a second exon for activation (FIG. 10A). Notably, co-transfection of constructs expressing the gRNA arrays and dCasRx-RBM25, followed by RT-PCR of the endogenous targeted exons, reveals simultaneous perturbations for all three tested pairs of exons (FIGS. 10B-10D). The order of the dCasRx array does not affect activation or repression efficiencies. Compared to single-targeting gRNAs, gRNA arrays have similarly high efficiencies in inducing exon skipping, while exon activation was slightly reduced. These results thus highlight the utility of dCasRx-RBM25 for generating combinatorial and bidirectional exon perturbations with high efficiency.

### Example 7

### Stable Multiplexed Activation of Endogenous Alternative Exons in HAP1 and HEK293T Cells

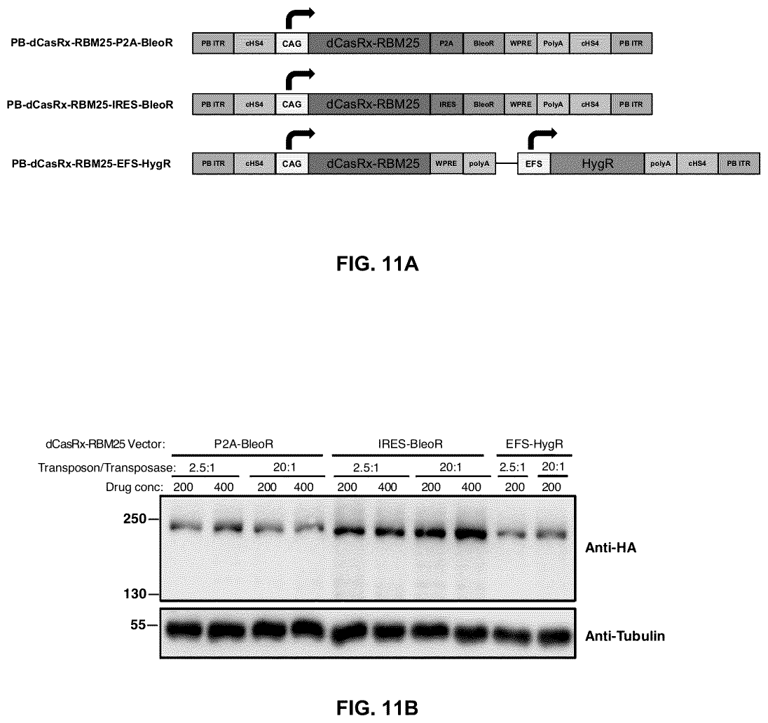

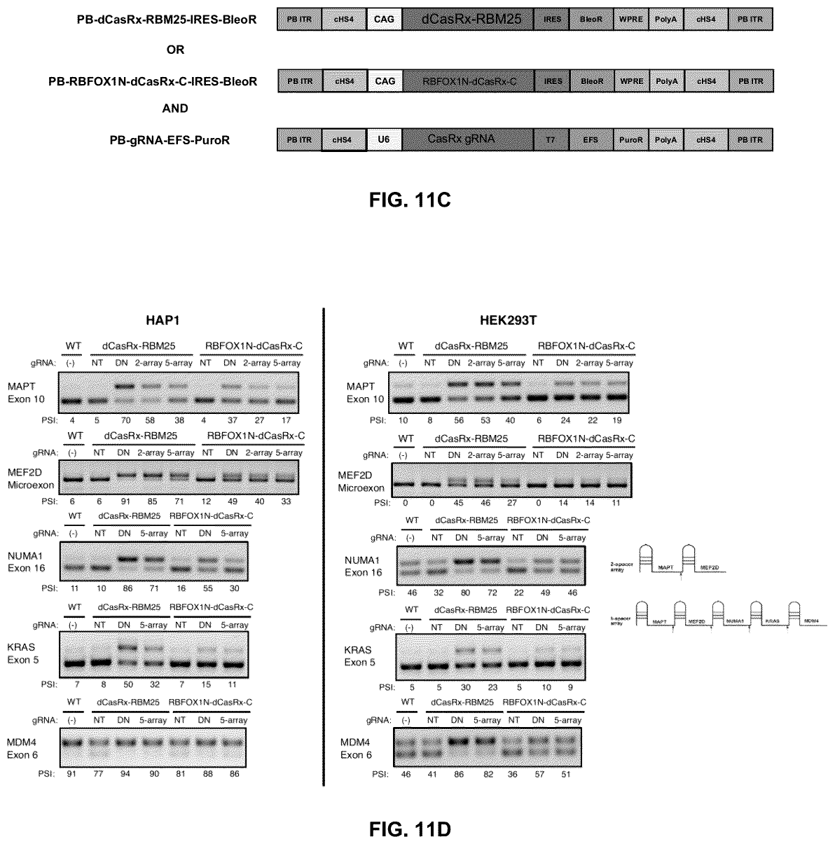

To assess the potential of dCasRx-RBM25 for long-term, stable activation of endogenous alternative exons, piggyBac (PB) transposon vectors were generated with the coding sequence of dCasRx-RBM25 and antibiotic selection cassettes flanked by inverted terminal repeats (ITRs) (FIG. 11A). Among three initial vector designs tested in HEK293T cells, following co-transfection with PB transposase and antibiotic selection, all three resulted in transgene expression with dCasRx-RBM25-IRES-BleoR yielded the highest level of stable transgene expression (FIG. 11B). To enable direct comparison of stable activation efficiencies, RBFOX1N-dCasRx-C was cloned into the same vector backbone. HEK293T or HAP1 cells were co-transfected with PB-dCasRx and PB-gRNA vectors, along with piggyBac transposase, and selected with puromycin and zeocin for two weeks to obtain polyclonal populations stably expressing both dCasRx and gRNAs (FIG. 11C). RT-PCR was subsequently used to assess endogenous splicing changes.

Reflecting previous results based on transient transfection, dCasRx-RBM25 outperforms RBFOX1N-dCasRx-C in the stable activation of all 5/5 tested targets using single downstream-targeting gRNAs across both HEK293T and HAP1 cell lines (FIG. 11D). In addition, it also shows markedly higher activation efficiencies when simultaneously targeting multiple exons for activation in either a two-spacer array (targeting MAPT exon 10 and MEF2D microexon) or a five-spacer array (targeting MAPT exon 10, MEF2D microexon, NUMA1 exon 16, KRAS exon 5, and MDM4 exon 6). These results thus establish transposon platforms such as the piggyBac-based delivery platform used here, as enabling efficient, stable long-term activation of endogenous exons using dCasRx-RBM25.

TABLE 1

| | Annotation- | | | | |
|---|---|---|---|---|---|
| | RNA | Annotation- | Annotation- | Clone | Protein |
| Gene | splicing | Spliceosome | Speckle | source | length |
| AAR2 | TRUE | TRUE | FALSE | 8.1 | 384 |
| ADAR | FALSE | TRUE | FALSE | OC | 1226 |
| AKAP17A | TRUE | TRUE | FALSE | 8.1 | 695 |
| ALDOC | FALSE | FALSE | TRUE | 8.1 | 364 |
| ALYREF | TRUE | TRUE | FALSE | OC | 257 |
| API5 | FALSE | TRUE | TRUE | OC | 504 |
| AURKA | FALSE | FALSE | TRUE | OC | 403 |
| BCAS2 | TRUE | TRUE | TRUE | 8.1 | 225 |
| BRDT | TRUE | FALSE | FALSE | OC | 947 |
| BUD13 | TRUE | TRUE | FALSE | 8.1 | 619 |
| BUD31 | TRUE | TRUE | FALSE | 8.1 | 144 |
| C1QBP | TRUE | FALSE | FALSE | 9.1 | 282 |
| C2orf49 | TRUE | FALSE | FALSE | 8.1 | 232 |
| C9orf78 | TRUE | TRUE | FALSE | 8.1 | 289 |
| CACTIN | TRUE | TRUE | FALSE | OC | 758 |
| CARM1 | FALSE | FALSE | TRUE | OC | 608 |
| CCAR2 | TRUE | FALSE | FALSE | 8.1 | 365 |
| CCDC12 | FALSE | TRUE | FALSE | 8.1 | 166 |
| CD2BP2 | TRUE | TRUE | FALSE | 8.1 | 341 |
| CDC40 | TRUE | TRUE | FALSE | 9.1 | 579 |
| CDC5L | TRUE | TRUE | TRUE | 9.1 | 802 |
| CDK10 | FALSE | TRUE | FALSE | 8.1 | 283 |
| CELF1 | TRUE | FALSE | FALSE | OC | 483 |
| CELF3 | TRUE | FALSE | FALSE | 8.1 | 464 |
| CELF4 | TRUE | FALSE | FALSE | 8.1 | 484 |
| CELF5 | TRUE | FALSE | FALSE | 8.1 | 485 |
| CELF6 | TRUE | FALSE | FALSE | 9.1 | 368 |
| CHMP5 | FALSE | FALSE | TRUE | 8.1 | 219 |
| CIR1 | TRUE | FALSE | FALSE | 8.1 | 202 |
| CIRBP | FALSE | TRUE | FALSE | 8.1 | 172 |
| CLASRP | TRUE | FALSE | FALSE | 9.1 | 659 |
| CLP1 | TRUE | FALSE | FALSE | 8.1 | 425 |
| COIL | TRUE | FALSE | FALSE | 9.1 | 576 |
| CPEB4 | FALSE | FALSE | FALSE | 9.1 | 729 |
| CPSF4 | FALSE | FALSE | FALSE | 8.1 | 244 |
| CPSF6 | FALSE | FALSE | TRUE | 8.1 | 588 |
| CRNKL1 | TRUE | TRUE | TRUE | OC | 848 |
| CWC15 | TRUE | TRUE | FALSE | 8.1 | 229 |
| CWC22 | TRUE | TRUE | FALSE | 8.1 | 908 |
| CWC27 | TRUE | TRUE | FALSE | 9.1 | 291 |
| CWF19L1 | TRUE | TRUE | FALSE | 8.1 | 538 |
| CXorf56 | FALSE | TRUE | FALSE | 8.1 | 222 |
| DAZAP1 | FALSE | FALSE | FALSE | 8.1 | 407 |
| DBR1 | TRUE | FALSE | FALSE | 8.1 | 544 |
| DCPS | TRUE | FALSE | FALSE | 8.1 | 337 |
| DDX1 | TRUE | FALSE | FALSE | 8.1 | 740 |
| DDX17 | TRUE | FALSE | FALSE | 8.1 | 650 |
| DDX20 | TRUE | FALSE | FALSE | 8.1 | 824 |
| DDX23 | TRUE | TRUE | TRUE | 8.1 | 820 |
| DDX39A | TRUE | FALSE | FALSE | 8.1 | 427 |
| DDX39B | TRUE | TRUE | FALSE | OC | 428 |
| DDX41 | TRUE | TRUE | TRUE | 8.1 | 622 |
| DDX47 | TRUE | FALSE | FALSE | 8.1 | 323 |
| DHX16 | TRUE | TRUE | FALSE | 8.1 | 1042 |
| DHX32 | FALSE | TRUE | FALSE | 8.1 | 743 |
| DHX36 | FALSE | TRUE | FALSE | 8.1 | 979 |
| DHX38 | TRUE | TRUE | FALSE | 8.1 | 1227 |
| DHX8 | TRUE | TRUE | FALSE | 8.1 | 1214 |
| DNAJC17 | TRUE | TRUE | FALSE | 8.1 | 304 |
| DQX1 | FALSE | TRUE | FALSE | 8.1 | 599 |
| ECD | TRUE | FALSE | FALSE | 8.1 | 644 |
| EEF1A1 | FALSE | TRUE | FALSE | 8.1 | 462 |
| EFTUD2 | TRUE | TRUE | TRUE | 8.1 | 972 |
| EIF4A3 | TRUE | TRUE | FALSE | 8.1 | 411 |
| ELAVL4 | TRUE | FALSE | FALSE | 8.1 | 366 |
| ERH | FALSE | TRUE | TRUE | 8.1 | 104 |
| ESRP1 | TRUE | FALSE | FALSE | 8.1 | 681 |
| ESS2 | TRUE | TRUE | FALSE | 8.1 | 476 |
| FAM172A | TRUE | FALSE | FALSE | 9.1 | 416 |
| FAM32A | FALSE | TRUE | FALSE | 8.1 | 112 |
| FAM50A | TRUE | TRUE | FALSE | OC | 339 |
| FAM50B | FALSE | TRUE | FALSE | 8.1 | 325 |
| FAM98A | TRUE | FALSE | FALSE | 8.1 | 518 |
| FMR1 | TRUE | FALSE | FALSE | 8.1 | 297 |
| FRG1 | TRUE | TRUE | FALSE | 8.1 | 258 |
| FXR1 | TRUE | FALSE | FALSE | OC | 539 |
| FXR2 | TRUE | FALSE | FALSE | 8.1 | 673 |
| GCFC2 | TRUE | TRUE | FALSE | 9.1 | 781 |
| GEMIN2 | TRUE | TRUE | FALSE | 8.1 | 280 |
| GEMIN4 | TRUE | FALSE | FALSE | OC | 1058 |
| GEMIN5 | TRUE | FALSE | FALSE | 9.1 | 1508 |
| GEMIN6 | TRUE | FALSE | FALSE | 8.1 | 167 |
| GEMIN7 | TRUE | FALSE | FALSE | 8.1 | 131 |
| GEMIN8 | TRUE | FALSE | FALSE | 8.1 | 242 |
| GPKOW | TRUE | TRUE | FALSE | 8.1 | 476 |
| HABP4 | TRUE | FALSE | FALSE | 9.1 | 308 |
| HNRNPA1 | TRUE | TRUE | FALSE | 8.1 | 320 |
| HNRNPA1L2 | TRUE | TRUE | FALSE | 9.1 | 320 |
| HNRNPA2B1 | TRUE | TRUE | FALSE | 8.1 | 249 |
| HNRNPC | TRUE | TRUE | FALSE | 8.1 | 306 |
| HNRNPDL | FALSE | TRUE | FALSE | 8.1 | 301 |
| HNRNPF | TRUE | TRUE | TRUE | 8.1 | 415 |
| HNRNPH1 | TRUE | TRUE | TRUE | 8.1 | 449 |
| HNRNPH2 | FALSE | FALSE | TRUE | 8.1 | 449 |
| HNRNPK | TRUE | TRUE | FALSE | 8.1 | 464 |
| HNRNPLL | FALSE | FALSE | FALSE | 9.1 | 542 |
| HNRNPR | TRUE | TRUE | FALSE | 8.1 | 636 |
| HNRNPU | TRUE | TRUE | FALSE | OC | 806 |
| HNRNPUL1 | FALSE | TRUE | FALSE | 9.1 | 856 |
| HOXB-AS3 | TRUE | FALSE | FALSE | 8.1 | 53 |
| HSPA8 | TRUE | TRUE | FALSE | 8.1 | 646 |
| IK | TRUE | TRUE | FALSE | 9.1 | 557 |
| ISY1 | TRUE | TRUE | TRUE | 8.1 | 285 |
| IWS1 | TRUE | FALSE | FALSE | 9.1 | 819 |
| JMJD6 | TRUE | FALSE | FALSE | 8.1 | 403 |
| KDM1A | TRUE | FALSE | FALSE | 9.1 | 730 |
| KHDC4 | TRUE | TRUE | FALSE | 8.1 | 241 |
| KHDRBS1 | TRUE | FALSE | FALSE | OC | 443 |
| KHDRBS2 | TRUE | FALSE | FALSE | 8.1 | 349 |
| KIN | FALSE | TRUE | FALSE | 9.1 | 393 |
| LARP7 | TRUE | FALSE | TRUE | 8.1 | 582 |
| LENG1 | FALSE | TRUE | FALSE | 8.1 | 264 |
| LGALS3 | TRUE | TRUE | FALSE | 8.1 | 250 |
| LSM1 | TRUE | FALSE | FALSE | 8.1 | 133 |
| LSM10 | TRUE | FALSE | FALSE | 8.1 | 123 |
| LSM2 | TRUE | TRUE | TRUE | 8.1 | 95 |
| LSM3 | TRUE | TRUE | TRUE | 8.1 | 102 |
| LSM4 | TRUE | TRUE | FALSE | 8.1 | 139 |
| LSM5 | TRUE | TRUE | FALSE | 8.1 | 91 |
| LSM7 | TRUE | TRUE | FALSE | 8.1 | 103 |
| LUC7L | TRUE | TRUE | FALSE | 8.1 | 354 |
| LUC7L2 | TRUE | TRUE | TRUE | 8.1 | 392 |
| MAGOH | TRUE | TRUE | FALSE | 8.1 | 146 |
| MAGOHB | TRUE | TRUE | FALSE | 9.1 | 148 |
| MBNL1 | TRUE | FALSE | FALSE | 8.1 | 382 |
| MBNL2 | TRUE | FALSE | FALSE | 8.1 | 367 |
| METTL14 | TRUE | FALSE | FALSE | 8.1 | 456 |
| MFAP1 | TRUE | TRUE | TRUE | 8.1 | 439 |
| MPHOSPH10 | TRUE | FALSE | FALSE | 9.1 | 681 |
| MSI1 | FALSE | FALSE | FALSE | OC | 362 |
| MSI2 | FALSE | FALSE | FALSE | 9.1 | 328 |
| MYEF2 | FALSE | TRUE | FALSE | 9.1 | 211 |
| NCBP1 | TRUE | TRUE | FALSE | 9.1 | 790 |
| NCBP2 | TRUE | TRUE | FALSE | 9.1 | 156 |
| NCL | FALSE | TRUE | FALSE | 8.1 | 482 |
| NONO | TRUE | FALSE | FALSE | 8.1 | 471 |
| NOSIP | FALSE | TRUE | FALSE | 8.1 | 301 |
| NOVA1 | TRUE | FALSE | FALSE | 8.1 | 507 |
| NOVA2 | TRUE | FALSE | FALSE | OC | 492 |
| NSRP1 | TRUE | TRUE | FALSE | OC | 558 |
| PABPN1 | FALSE | FALSE | TRUE | 9.1 | 306 |
| PAXBP1 | TRUE | TRUE | FALSE | OC | 917 |
| PCBP1 | FALSE | TRUE | FALSE | 8.1 | 356 |
| PCBP4 | FALSE | FALSE | FALSE | 8.1 | 360 |
| PHF5A | TRUE | TRUE | FALSE | 8.1 | 110 |
| PHPT1 | FALSE | FALSE | TRUE | 8.1 | 125 |
| PLRG1 | TRUE | TRUE | FALSE | 8.1 | 505 |
| PNN | TRUE | TRUE | FALSE | 8.1 | 717 |
| POLDIP3 | FALSE | FALSE | TRUE | 8.1 | 229 |
| POLR2A | FALSE | FALSE | TRUE | 8.1 | 566 |
| PPARGC1A | TRUE | FALSE | FALSE | OC | 798 |
| PPIE | TRUE | TRUE | FALSE | 8.1 | 301 |
| PPIG | TRUE | TRUE | FALSE | 8.1 | 739 |
| PPIH | TRUE | TRUE | FALSE | 9.1 | 177 |
| PPIL1 | TRUE | TRUE | FALSE | 8.1 | 166 |
| PPIL2 | FALSE | TRUE | FALSE | 8.1 | 527 |
| PPIL3 | TRUE | TRUE | FALSE | 8.1 | 161 |
| PPIL4 | FALSE | TRUE | FALSE | 8.1 | 492 |
| PPP1R8 | TRUE | TRUE | FALSE | 8.1 | 209 |
| PPP2CA | TRUE | FALSE | FALSE | 9.1 | 309 |
| PPP2R1A | TRUE | FALSE | FALSE | 8.1 | 589 |
| PPP4R2 | TRUE | FALSE | FALSE | 8.1 | 417 |
| PPWD1 | TRUE | TRUE | FALSE | 8.1 | 646 |
| PQBP1 | TRUE | TRUE | FALSE | 8.1 | 265 |
| PRCC | FALSE | TRUE | FALSE | 8.1 | 491 |
| PRKRIP1 | TRUE | TRUE | FALSE | 9.1 | 184 |
| PRMT5 | TRUE | FALSE | FALSE | OC | 637 |
| PRPF18 | TRUE | TRUE | FALSE | 9.1 | 342 |
| PRPF19 | TRUE | TRUE | TRUE | 8.1 | 504 |
| PRPF31 | TRUE | TRUE | TRUE | 8.1 | 499 |
| PRPF38A | TRUE | TRUE | TRUE | 8.1 | 125 |
| PRPF38B | TRUE | TRUE | FALSE | 8.1 | 190 |
| PRPF39 | TRUE | TRUE | FALSE | 8.1 | 629 |
| PRPF4 | TRUE | TRUE | TRUE | 8.1 | 522 |
| PRPF40A | TRUE | TRUE | TRUE | 8.1 | 215 |
| PRPF40B | TRUE | TRUE | FALSE | 8.1 | 871 |
| PRPF4B | TRUE | TRUE | TRUE | 9.1 | 1007 |
| PRPF6 | TRUE | TRUE | TRUE | 8.1 | 941 |
| PSMB4 | FALSE | FALSE | TRUE | 8.1 | 264 |
| PSME3 | FALSE | FALSE | TRUE | 8.1 | 254 |
| PTBP1 | TRUE | FALSE | FALSE | 9.1 | 557 |
| PTBP2 | TRUE | TRUE | FALSE | 9.1 | 532 |
| PTBP3 | TRUE | FALSE | FALSE | 8.1 | 558 |
| PUF60 | TRUE | TRUE | TRUE | 8.1 | 542 |
| PUS1 | TRUE | FALSE | FALSE | OC | 399 |
| PUS7 | TRUE | FALSE | FALSE | 8.1 | 259 |
| QKI | TRUE | FALSE | FALSE | 8.1 | 341 |
| RBM10 | TRUE | TRUE | TRUE | 8.1 | 930 |
| RBM11 | TRUE | FALSE | FALSE | 8.1 | 281 |
| RBM14 | TRUE | FALSE | FALSE | 8.1 | 669 |
| RBM15B | TRUE | FALSE | FALSE | 8.1 | 563 |
| RBM17 | TRUE | TRUE | TRUE | 8.1 | 401 |
| RBM19 | TRUE | FALSE | FALSE | 8.1 | 960 |
| RBM23 | TRUE | FALSE | FALSE | 8.1 | 424 |
| RBM24 | TRUE | FALSE | FALSE | 8.1 | 191 |
| RBM25 | TRUE | TRUE | TRUE | 8.1 | 843 |
| RBM28 | TRUE | TRUE | FALSE | 8.1 | 759 |
| RBM3 | FALSE | TRUE | FALSE | 8.1 | 157 |
| RBM38 | TRUE | FALSE | FALSE | OC | 216 |
| RBM4 | TRUE | FALSE | FALSE | 8.1 | 364 |
| RBM41 | TRUE | TRUE | FALSE | 9.1 | 413 |
| RBM42 | TRUE | TRUE | FALSE | 8.1 | 480 |
| RBM4B | TRUE | FALSE | FALSE | 9.1 | 359 |
| RBM5 | TRUE | TRUE | FALSE | 8.1 | 546 |
| RBM6 | TRUE | FALSE | FALSE | 8.1 | 601 |
| RBM7 | TRUE | FALSE | FALSE | 8.1 | 266 |
| RBM8A | TRUE | TRUE | TRUE | 9.1 | 174 |
| RBMX | TRUE | TRUE | FALSE | 8.1 | 391 |
| RBMX2 | TRUE | TRUE | FALSE | 8.1 | 322 |
| RBMXL2 | TRUE | TRUE | FALSE | 9.1 | 392 |
| RBMY1A1 | TRUE | FALSE | FALSE | 9.1 | 459 |
| RBMY1E | TRUE | FALSE | FALSE | OC | 496 |
| RBMY1F | TRUE | FALSE | FALSE | 8.1 | 496 |
| RBPMS2 | FALSE | FALSE | FALSE | OC | 209 |
| RHEB | FALSE | TRUE | FALSE | 8.1 | 184 |
| RNF113A | TRUE | TRUE | FALSE | 8.1 | 343 |
| RNPC3 | TRUE | TRUE | FALSE | 8.1 | 261 |
| RNPS1 | TRUE | FALSE | TRUE | 8.1 | 305 |
| RP9 | TRUE | FALSE | FALSE | OC | 221 |
| RPRD1B | FALSE | FALSE | TRUE | 8.1 | 326 |
| RPSA | FALSE | TRUE | FALSE | 8.1 | 295 |
| RPUSD4 | TRUE | FALSE | FALSE | 8.1 | 377 |
| RRAGC | TRUE | FALSE | FALSE | 8.1 | 399 |
| RSRC1 | TRUE | FALSE | FALSE | 8.1 | 334 |
| RTCB | TRUE | FALSE | FALSE | 9.1 | 505 |
| RTRAF | TRUE | FALSE | FALSE | 9.1 | 244 |
| RUVBL2 | FALSE | TRUE | FALSE | 8.1 | 463 |
| SAP18 | TRUE | TRUE | TRUE | 8.1 | 153 |
| SAP30BP | FALSE | FALSE | TRUE | 8.1 | 308 |
| SART1 | TRUE | TRUE | TRUE | OC | 800 |
| SART3 | TRUE | FALSE | FALSE | 8.1 | 963 |
| SCAF11 | TRUE | FALSE | FALSE | OC | 1463 |
| SCNM1 | TRUE | FALSE | FALSE | 8.1 | 230 |
| SF1 | TRUE | TRUE | FALSE | 8.1 | 548 |
| SF3A2 | TRUE | TRUE | FALSE | OC | 464 |
| SF3A3 | TRUE | TRUE | TRUE | 9.1 | 501 |
| SF3B2 | TRUE | TRUE | TRUE | 8.1 | 636 |
| SF3B3 | TRUE | TRUE | TRUE | 8.1 | 399 |
| SF3B4 | TRUE | TRUE | TRUE | 8.1 | 424 |
| SF3B5 | TRUE | TRUE | FALSE | OC | 86 |
| SFPQ | TRUE | FALSE | FALSE | 8.1 | 707 |
| SFSWAP | TRUE | FALSE | FALSE | 8.1 | 951 |
| SLU7 | TRUE | TRUE | FALSE | 8.1 | 586 |
| SMN1 | TRUE | FALSE | FALSE | OC | 282 |
| SMNDC1 | TRUE | TRUE | FALSE | 8.1 | 238 |
| SMU1 | TRUE | TRUE | TRUE | 8.1 | 513 |
| SNIP1 | TRUE | TRUE | FALSE | 8.1 | 396 |
| SNRNP25 | TRUE | TRUE | FALSE | 9.1 | 132 |
| SNRNP27 | TRUE | TRUE | FALSE | 8.1 | 155 |
| SNRNP40 | TRUE | TRUE | FALSE | 8.1 | 357 |
| SNRNP48 | TRUE | TRUE | FALSE | 8.1 | 339 |
| SNRNP70 | TRUE | TRUE | TRUE | 8.1 | 437 |
| SNRPA | TRUE | TRUE | FALSE | 8.1 | 282 |
| SNRPA1 | TRUE | TRUE | TRUE | 8.1 | 255 |
| SNRPB | TRUE | TRUE | FALSE | 8.1 | 231 |
| SNRPB2 | TRUE | TRUE | FALSE | 8.1 | 225 |
| SNRPD1 | TRUE | TRUE | FALSE | 9.1 | 119 |
| SNRPD2 | TRUE | TRUE | FALSE | 8.1 | 118 |
| SNRPD3 | TRUE | TRUE | TRUE | OC | 126 |
| SNRPE | TRUE | TRUE | FALSE | 8.1 | 92 |
| SNRPF | TRUE | TRUE | FALSE | 8.1 | 86 |
| SNRPG | TRUE | TRUE | FALSE | 8.1 | 76 |
| SNRPN | TRUE | TRUE | FALSE | 8.1 | 240 |
| SNU13 | TRUE | TRUE | FALSE | 8.1 | 128 |
| SNW1 | TRUE | TRUE | TRUE | 8.1 | 536 |
| SREK1 | TRUE | TRUE | FALSE | 8.1 | 508 |
| SREK1IP1 | TRUE | FALSE | FALSE | 8.1 | 155 |
| SRPK1 | TRUE | FALSE | FALSE | 8.1 | 655 |
| SRPK2 | TRUE | FALSE | FALSE | 8.1 | 688 |
| SRPK3 | TRUE | FALSE | FALSE | 8.1 | 566 |
| SRRM4 | TRUE | FALSE | FALSE | OC | 611 |
| SRRT | FALSE | TRUE | TRUE | 9.1 | 871 |
| SRSF1 | TRUE | TRUE | TRUE | 8.1 | 248 |
| SRSF10 | TRUE | TRUE | FALSE | 8.1 | 262 |
| SRSF11 | TRUE | FALSE | FALSE | 8.1 | 483 |
| SRSF12 | TRUE | FALSE | FALSE | 8.1 | 261 |
| SRSF2 | TRUE | FALSE | TRUE | 8.1 | 179 |
| SRSF3 | TRUE | FALSE | FALSE | 8.1 | 164 |
| SRSF4 | TRUE | FALSE | FALSE | 8.1 | 494 |
| SRSF5 | TRUE | FALSE | FALSE | 8.1 | 272 |
| SRSF6 | TRUE | TRUE | TRUE | 8.1 | 344 |
| SRSF7 | TRUE | TRUE | TRUE | 8.1 | 238 |
| SRSF8 | TRUE | FALSE | FALSE | 8.1 | 275 |
| SRSF9 | TRUE | TRUE | TRUE | 8.1 | 221 |
| STRAP | TRUE | FALSE | FALSE | 8.1 | 350 |
| SUGP1 | TRUE | TRUE | FALSE | 8.1 | 645 |
| SYF2 | TRUE | TRUE | FALSE | 8.1 | 243 |
| SYNCRIP | TRUE | TRUE | FALSE | 8.1 | 527 |
| TAF15 | TRUE | FALSE | FALSE | 8.1 | 592 |
| TFIP11 | TRUE | TRUE | TRUE | 8.1 | 837 |
| THOC1 | TRUE | FALSE | FALSE | 8.1 | 657 |
| THOC3 | TRUE | FALSE | FALSE | 8.1 | 351 |
| THOC5 | TRUE | TRUE | FALSE | 8.1 | 683 |
| THOC7 | TRUE | FALSE | FALSE | 8.1 | 204 |
| THRAP3 | TRUE | TRUE | FALSE | 8.1 | 955 |
| TIA1 | TRUE | FALSE | FALSE | 9.1 | 214 |
| TLE3 | FALSE | FALSE | TRUE | 8.1 | 772 |
| TRA2A | TRUE | TRUE | TRUE | 8.1 | 282 |
| TRA2B | TRUE | TRUE | TRUE | OC | 288 |
| TRPT1 | TRUE | FALSE | FALSE | 8.1 | 204 |
| TSEN15 | TRUE | FALSE | FALSE | 8.1 | 171 |
| TSEN2 | TRUE | FALSE | FALSE | 8.1 | 465 |
| TSEN54 | TRUE | FALSE | FALSE | 9.1 | 526 |
| TSSC4 | TRUE | FALSE | FALSE | 8.1 | 329 |
| TTF2 | TRUE | TRUE | FALSE | 8.1 | 676 |
| TUBA1A | FALSE | TRUE | FALSE | 8.1 | 451 |
| TUBB | FALSE | TRUE | FALSE | 8.1 | 444 |
| TXNL4A | TRUE | TRUE | FALSE | 8.1 | 142 |
| TXNL4B | TRUE | TRUE | FALSE | 8.1 | 149 |
| U2AF1 | TRUE | TRUE | TRUE | 8.1 | 240 |
| U2AF1L4 | TRUE | TRUE | FALSE | 8.1 | 202 |
| U2AF2 | TRUE | TRUE | TRUE | 8.1 | 471 |
| U2SURP | FALSE | TRUE | TRUE | 8.1 | 620 |
| UBL5 | TRUE | TRUE | FALSE | 8.1 | 73 |
| USB1 | TRUE | FALSE | FALSE | 8.1 | 265 |
| USP39 | TRUE | TRUE | FALSE | 8.1 | 565 |
| USP4 | TRUE | FALSE | FALSE | 8.1 | 963 |
| USP49 | TRUE | FALSE | FALSE | 8.1 | 640 |
| WAC | FALSE | TRUE | FALSE | 8.1 | 649 |
| WBP11 | TRUE | TRUE | FALSE | 8.1 | 641 |
| WBP4 | TRUE | TRUE | FALSE | 8.1 | 376 |
| WDR83 | TRUE | TRUE | FALSE | 8.1 | 315 |
| WT1 | TRUE | FALSE | FALSE | 8.1 | 302 |
| WTAP | TRUE | FALSE | TRUE | 9.1 | 396 |
| YBX1 | TRUE | TRUE | FALSE | OC | 324 |
| YJU2 | TRUE | TRUE | FALSE | 8.1 | 323 |
| ZBTB8OS | TRUE | FALSE | FALSE | 8.1 | 136 |
| ZC3H11A | FALSE | FALSE | TRUE | 8.1 | 810 |
| ZC3H14 | FALSE | FALSE | TRUE | 8.1 | 306 |
| ZC3H18 | FALSE | TRUE | FALSE | 8.1 | 953 |
| ZCCHC8 | TRUE | TRUE | FALSE | 9.1 | 707 |
| ZCRB1 | TRUE | TRUE | FALSE | 8.1 | 217 |
| ZFP36 | FALSE | FALSE | FALSE | 9.1 | 326 |
| ZMAT2 | TRUE | TRUE | FALSE | 8.1 | 199 |
| ZMAT5 | TRUE | TRUE | FALSE | 8.1 | 170 |
| ZNF12 | FALSE | FALSE | FALSE | 9.1 | 697 |
| ZNF326 | TRUE | FALSE | FALSE | 9.1 | 582 |
| ZNF830 | TRUE | TRUE | FALSE | 8.1 | 348 |
| ZPR1 | TRUE | FALSE | FALSE | 9.1 | 459 |
| ZRANB2 | TRUE | FALSE | FALSE | 8.1 | 320 |
| ZRSR2 | TRUE | TRUE | FALSE | 8.1 | 482 |

TABLE 2

| | Log2FC - | | Log2FC - | | | |
|---|---|---|---|---|---|---|
| | SMN2 single | FDR - SMN2 | SMN2 triple | FDR - SMN2 | Log2FC - | |
| Construct | gRNA | single gRNA | gRNA | triple gRNA | Mef2d | FDR - Mef2d |
| AAR2 | 0.04225141 | 0.66413381 | 0.04607587 | 0.46097809 | −0.0542957 | 0.03275622 |
| ADAR | −0.0047807 | 0.94231579 | −0.1733189 | 0.67208148 | −0.0279531 | 0.21030938 |
| AKAP17A | −0.3949396 | 0.12792165 | −0.2930976 | 0.00286594 | 0.10823236 | 0.42401474 |
| ALDOC | 0.12295616 | 0.49256804 | 0.1631935 | 0.23997236 | 0.50247363 | 0.58913448 |
| ALYREF | −0.1905481 | 0.06253624 | −0.3346934 | 0.02474781 | −0.1887897 | 0.01340919 |
| API5 | 0.09660732 | 0.11496732 | −0.0778566 | 1 | 0.18809698 | 0.15030412 |
| AURKA | −0.0961716 | 0.20598173 | −0.1025205 | 0.8173306 | 0.46976243 | 0.6021751 |
| BCAS2 | 0.0711506 | 0.44645536 | 0.05940605 | 0.55555731 | 0.76591859 | 0.11340497 |
| BRDT | 0.24806618 | 0.2657474 | 0.14109082 | 0.1976214 | −0.1659206 | 0.14893387 |
| BUD13 | 0.17628936 | 0.20353132 | 0.05082575 | 0.34433938 | 0.69264552 | 0.11226298 |
| BUD31 | −0.0602462 | 0.88898191 | −0.1033729 | 0.93188151 | 0.68591575 | 0.0182099 |
| C1QBP | −0.0429877 | 0.98666122 | −0.0619263 | 0.82250388 | 0.14732782 | 0.21106434 |
| C2orf49 | −0.0672288 | 0.92005625 | −0.0808717 | 1 | 0.2059925 | 0.04492006 |
| C9orf78 | 0.15294934 | 0.39624519 | 0.00847334 | 0.59218802 | 0.59401285 | 0.17497234 |
| CACTIN | −0.1708539 | 0.20353132 | −0.0098616 | 0.29283193 | 0.9666975 | 0.0029275 |
| CARM1 | 0.09792852 | 0.30530724 | 0.23129983 | 0.01812399 | 1.4243554 | 0.00771527 |
| CCAR2 | 0.0608361 | 0.37396257 | 0.09594973 | 0.03433185 | −0.0206841 | 0.054003 |
| CCDC12 | 0.0744292 | 0.68277778 | −0.0225781 | 0.55555731 | 0.15051686 | 0.03667933 |
| CD2BP2 | 0.24024262 | 0.22918973 | 0.28248702 | 0.0297968 | 0.5261211 | 0.59340867 |
| CDC40 | 0.1398831 | 0.04767552 | 0.03935838 | 0.54637289 | 0.97918236 | 0.0608439 |
| CDC5L | −0.0857309 | 0.85479317 | −0.00571 | 0.79454347 | 0.09834643 | 0.22653393 |
| CDK10 | 0.11073804 | 0.30602287 | 0.1365252 | 0.02309854 | 0.21126175 | 0.2949648 |
| CELF1 | 0.22677281 | 0.49715958 | 0.52825288 | 0.04641511 | −0.2800154 | 0.0024824 |
| CELF3 | 0.86148049 | 0.03986434 | 1.77014989 | 1.47E−12 | −0.2174991 | 0.11162208 |
| CELF4 | 0.41969213 | 0.00188983 | 0.57493134 | 0.0003463 | 0.06715978 | 0.00063588 |
| CELF5 | 0.43787175 | 0.24153906 | 0.8388092 | 0.0000373 | 0.0014391 | 0.0008816 |
| CELF6 | 0.45133314 | 0.24280862 | 0.82673201 | 0.00189538 | 0.63787461 | 0.17991142 |
| CHMP5 | −0.1400005 | 0.54928808 | 0.19955721 | 0.00031911 | −0.017033 | 0.03275622 |
| CIR1 | 0.09096852 | 0.48349462 | 0.00225911 | 0.70519712 | 0.74569846 | 0.01811503 |
| CIRBP | −0.0289939 | 0.98666122 | −0.0922931 | 0.98685789 | −0.4034431 | 8.73E−08 |
| CLASRP | 0.10757279 | 0.53741673 | 0.12066027 | 0.39173954 | 0.8539115 | 0.11874683 |
| CLP1 | 0.15432719 | 0.39276716 | −0.0114326 | 0.55555731 | 0.462303 | 0.40637223 |
| COIL | −0.0013783 | 0.88898191 | 0.05797931 | 0.3071572 | −0.1577598 | 0.12188283 |
| CPEB4 | 0.04207975 | 0.22262511 | 0.25261626 | 0.03433185 | 0.35491357 | 0.8238125 |
| CPSF4 | −0.0022013 | 0.49715958 | 0.15942727 | 0.00126524 | 0.30151792 | 0.25931165 |
| CPSF6 | 0.09807233 | 0.48349462 | 0.70173909 | 0.00000071 | 0.24334066 | 0.17991142 |
| CRNKL1 | −0.1326511 | 0.33749431 | −0.0196885 | 0.44286055 | 0.16907623 | 0.00909662 |
| CWC15 | 0.16066955 | 0.39624519 | −0.033489 | 0.8571649 | 1.31186954 | 0.06371242 |
| CWC22 | 0.07934449 | 0.6205953 | 0.11557519 | 0.29729511 | 0.81830278 | 0.0000415 |
| CWC27 | −0.0418577 | 0.98666122 | −0.0896699 | 0.98132018 | 0.52249827 | 0.21537095 |
| CWF19L1 | 0.00756901 | 0.66413381 | 0.08878698 | 0.1976214 | 0.23327647 | 0.64531406 |
| CXorf56 | 0.05784663 | 0.06497545 | −0.0724597 | 0.98747288 | 0.51862128 | 0.16498642 |
| DAZAP1 | 0.14066709 | 0.00069524 | 0.19220658 | 0.19514483 | 0.36176131 | 0.83936588 |
| DBR1 | −0.0242421 | 0.98666122 | −0.0711187 | 0.94155346 | 0.38130669 | 0.99444321 |
| dCasRx | −0.0366739 | 1 | −0.0800695 | 1 | 0.38327191 | 1 |
| DCPS | −0.038199 | 1 | −0.0053244 | 0.72098789 | 0.36805896 | 0.8630899 |
| DDX1 | 0.0162771 | 0.63968156 | −0.1290248 | 0.93270579 | 0.33513714 | 0.8581711 |
| DDX17 | −0.0032334 | 0.90929249 | −0.0511698 | 0.93270579 | −0.1514439 | 0.00258695 |
| DDX20 | 0.07059226 | 0.54579518 | 0.13880767 | 0.05161602 | 0.59968103 | 0.00831455 |
| DDX23 | 0.01475688 | 0.79428618 | 0.05933945 | 0.07034084 | 1.09308754 | 0.08236859 |
| DDX39A | 0.12957344 | 0.53211721 | 0.30329945 | 0.03110346 | 0.05218731 | 0.08053038 |
| DDX39B | 0.11568287 | 0.44645536 | 0.2014205 | 0.19514483 | −0.153076 | 0.175997 |
| DDX41 | −2.83E−05 | 0.75919928 | 0.02416795 | 0.40946955 | 0.47410132 | 0.6021751 |
| DDX47 | 0.04879074 | 0.47835434 | −0.0167444 | 0.79041771 | 0.39456721 | 0.96859683 |
| DHX16 | −0.0095612 | 0.90929249 | −0.0717346 | 0.97921648 | 0.66351985 | 0.01834362 |
| DHX32 | −0.0679141 | 0.88618385 | −0.1440957 | 0.55228006 | 0.26819545 | 0.50149745 |
| DHX36 | 0.09026512 | 0.39624519 | −0.124765 | 0.85330301 | 0.32372701 | 0.64757017 |
| DHX38 | −0.0357266 | 1 | −0.1966239 | 0.38756349 | 0.56444581 | 0.37501264 |
| DHX8 | 0.15639562 | 0.25207696 | 0.13955974 | 0.33528672 | 0.90321961 | 0.25408369 |
| DNAJC17 | −0.0107862 | 0.76952991 | −0.0166137 | 0.71779179 | 0.59280137 | 0.17982577 |
| DQX1 | 0.04364786 | 0.61556347 | −0.0824176 | 1 | −0.0463214 | 0.12907191 |
| ECD | 0.07677825 | 0.44645536 | 0.07888842 | 0.21479538 | 0.15067622 | 0.37733256 |
| EEF1A1 | 0.01193301 | 0.78625244 | 0.0385945 | 0.20547751 | 0.53156112 | 0.25408369 |
| EFTUD2 | 0.0865861 | 0.27809631 | 0.03468799 | 0.43900295 | 0.33558507 | 0.64793631 |
| EGFP | −0.0520834 | 0.82857137 | −0.1280639 | 0.63966648 | 0.39340583 | 0.95634055 |
| EIF4A3 | −0.0861556 | 0.54579518 | −0.0052039 | 0.52700562 | 0.54513887 | 0.43061921 |
| ELAVL4 | 0.21864899 | 1.64E−05 | 0.15877898 | 0.08600664 | −0.0814825 | 0.00056414 |
| ERH | 0.02081332 | 0.85559451 | 0.05837997 | 0.41985536 | 0.56171098 | 0.45205084 |
| ESRP1 | 0.49943198 | 0.00105038 | 0.46019965 | 0.03419713 | 0.94635907 | 0.01084609 |
| ESS2 | 0.02262863 | 0.53741673 | −0.0019301 | 0.48372223 | 0.48459526 | 0.5582617 |
| FAM172A | −0.1208952 | 0.55297429 | −0.0594113 | 0.8173306 | 0.21167848 | 0.24497251 |
| FAM32A | 0.18307291 | 0.28381499 | 0.1239288 | 0.30840531 | 0.52573861 | 0.40637223 |
| FAM50A | 0.05361167 | 0.66413381 | 0.026513 | 0.57896619 | 0.3199074 | 0.62560698 |
| FAM50B | 0.02105068 | 0.29950177 | 0.0407789 | 0.60426641 | 0.43819609 | 0.53490998 |
| FAM98A | −0.0370675 | 1 | −0.0681578 | 0.98932229 | −0.2237937 | 0.0000955 |
| FMR1 | 0.01179925 | 0.90757939 | −0.0140663 | 0.78160333 | 0.54889711 | 0.03440699 |
| FRG1 | −0.0711037 | 0.8577371 | −0.0624903 | 0.97431847 | 0.27443734 | 0.51708013 |
| FXR1 | 0.08014122 | 0.33749431 | 0.1322246 | 0.30840531 | 0.16202378 | 0.04879317 |
| FXR2 | 0.09344015 | 0.63709814 | 0.09095818 | 0.44286055 | 0.2378478 | 0.33464768 |
| GCFC2 | 0.01129183 | 0.85233573 | 0.07617238 | 0.30840531 | 0.28169527 | 0.17497234 |
| GEMIN2 | 0.19365379 | 0.26876189 | 0.06740906 | 0.63660713 | 0.56237795 | 0.17094169 |
| GEMIN4 | 0.08131459 | 0.30602287 | 0.05776153 | 0.12822531 | 0.414367 | 0.76718294 |
| GEMIN5 | 0.03349438 | 0.39624519 | −0.0147467 | 0.55228006 | 0.47814664 | 0.2428708 |
| GEMIN6 | 0.07983991 | 0.31553334 | 0.02108822 | 0.67622972 | 0.40757756 | 0.91135579 |
| GEMIN7 | 0.07535199 | 0.57069774 | −0.116343 | 0.71779179 | 0.52760633 | 0.55587185 |
| GEMIN8 | 0.04120317 | 0.63200801 | 0.03674408 | 0.56931211 | 0.89154261 | 0.11922265 |
| GPKOW | 0.0224183 | 0.68277778 | 0.02764989 | 0.58640145 | 0.92722375 | 0.00049927 |
| HABP4 | 0.00056261 | 0.86492924 | −0.0232816 | 0.77178027 | 0.71240041 | 0.1194788 |
| HNRNPA1 | 0.1869832 | 0.00249383 | 0.48128508 | 0.00654674 | 0.19420023 | 0.32966061 |
| HNRNPA1L2 | −0.0034721 | 0.64492093 | 0.10625844 | 0.11178122 | 0.11763965 | 0.00685661 |
| HNRNPA2B1 | 0.04792496 | 0.24280862 | 0.02477164 | 0.47439371 | 0.38584117 | 0.99444321 |
| HNRNPC | −0.1132792 | 0.44645536 | 0.12052337 | 0.44473847 | 0.44371406 | 0.41373642 |
| HNRNPDL | −0.1578564 | 0.33749431 | −0.4107051 | 0.08600664 | 0.22461441 | 0.62619915 |
| HNRNPF | 0.48188294 | 0.00926773 | 0.60417093 | 0.12059638 | 0.06538076 | 0.15030412 |
| HNRNPH1 | 0.39781003 | 0.16101498 | 0.3486054 | 0.00085564 | −0.1513326 | 0.000034 |
| HNRNPH2 | 0.33832292 | 0.17084255 | 0.46422939 | 0.00189022 | −0.1438953 | 0.01896468 |
| HNRNPK | 0.27565166 | 0.00032314 | 0.30965298 | 0.02677993 | −0.2254006 | 0.01606372 |
| HNRNPLL | 0.39108987 | 0.15203687 | 0.44337792 | 0.0585331 | 0.24413144 | 0.25663674 |
| HNRNPR | −0.028811 | 0.98666122 | −0.2359707 | 0.67372637 | −0.2016887 | 0.00000225 |
| HNRNPU | −0.0286471 | 0.98666122 | −0.1300738 | 0.70961205 | −0.0175776 | 0.25931165 |
| HNRNPUL1 | 0.06533857 | 0.6243137 | 0.06524256 | 0.02677993 | 0.2741909 | 0.66012211 |
| HOXB-AS3 | 0.10744301 | 0.33749431 | 0.0992452 | 0.17274968 | 0.94544363 | 0.00000326 |
| HSPA8 | −0.0763466 | 0.6963554 | −0.1055036 | 0.97431847 | 0.35666533 | 0.94579615 |
| IK | 0.0362981 | 0.55297429 | 0.00507128 | 0.56668068 | 0.35521169 | 0.80225835 |
| ISY1 | 0.09588379 | 0.44645536 | 0.03999046 | 0.58540742 | 0.39270257 | 0.98705 |
| IWS1 | 0.05622962 | 0.31553334 | −0.0123367 | 0.72662236 | −0.1014379 | 0.00020528 |
| JMJD6 | 0.11225248 | 0.08500988 | 0.11148772 | 0.39173954 | 0.54879954 | 0.40443648 |
| KDM1A | 0.07313033 | 0.59307664 | 0.2108473 | 0.30840531 | 0.43384358 | 0.81610568 |
| KHDC4 | −0.1840995 | 0.09680977 | 0.45093366 | 0.00000527 | 2.34498064 | 0.0153777 |
| KHDRBS1 | 0.11862251 | 0.55297429 | −0.0187923 | 0.44473847 | −0.4258447 | 6.86E−08 |
| KHDRBS2 | 0.01569823 | 0.79428618 | 0.03386275 | 0.39762866 | −0.3710538 | 0.00145799 |
| KIN | 0.12993768 | 0.39017084 | −0.0149284 | 0.71284149 | 0.62069729 | 0.51249735 |
| LARP7 | 0.04665247 | 0.49052783 | −0.1014976 | 0.93270579 | 0.12678661 | 0.00292288 |
| LENG1 | 0.1220609 | 0.4504789 | −0.0851262 | 0.99633225 | 0.62352569 | 0.06029074 |
| LGALS3 | 0.07016218 | 0.09680977 | 0.14578908 | 0.12078002 | 1.05007364 | 0.08930875 |
| LSM1 | 0.00742995 | 0.55297429 | −0.0176435 | 0.71461167 | 0.61585524 | 0.57363811 |
| LSM10 | −0.0064182 | 0.82857137 | −0.1399856 | 0.72753124 | 0.75268334 | 0.01084609 |
| LSM2 | 0.03192749 | 0.63200801 | −0.0886815 | 0.99573033 | 0.28667979 | 0.31460852 |
| LSM3 | 0.07127974 | 0.48349462 | −0.1186295 | 0.90679361 | 0.59260658 | 0.33079051 |
| LSM4 | −0.088466 | 0.70538977 | −0.1712578 | 0.63966648 | 0.39465946 | 0.94817586 |
| LSM5 | 0.15721036 | 0.57619157 | 0.06373453 | 0.44473847 | 0.22668555 | 0.56725594 |
| LSM7 | 0.12008461 | 0.18926023 | 0.1366881 | 0.35195344 | 0.75950849 | 0.00071724 |
| LUC7L | −0.1033286 | 0.63200801 | 0.04854572 | 0.55555731 | 1.18150655 | 0.13956107 |
| LUC7L2 | −0.0247787 | 0.88898191 | 0.21102081 | 0.24401239 | 1.51753202 | 0.03748194 |
| MAGOH | 0.03233662 | 0.66413381 | −0.0458098 | 0.8173306 | 0.48277937 | 0.62251113 |
| MAGOHB | −0.0401202 | 1 | 0.03471397 | 0.40322847 | 0.38854195 | 0.99444321 |
| MBNL1 | −0.1485556 | 0.63709814 | −0.0631592 | 0.97431847 | 0.37740518 | 0.99444321 |
| MBNL2 | 0.00886285 | 0.91471006 | 0.05361374 | 0.33693884 | 0.24340253 | 0.20904174 |
| METTL14 | 0.07544358 | 0.05695394 | 0.12102596 | 0.30840531 | 0.68174401 | 0.24136009 |
| MFAP1 | 0.00367025 | 0.63200801 | −0.0109202 | 0.54637289 | 0.1783346 | 0.47158482 |
| MPHOSPH10 | 0.04164034 | 0.64149828 | 0.06952977 | 0.55555731 | −0.1027299 | 0.07705102 |
| MSI1 | −0.0504857 | 0.98666122 | 0.16482728 | 0.38756349 | −0.1097583 | 0.08901058 |
| MSI2 | 0.0989614 | 0.77618493 | 0.2606906 | 0.23897545 | −0.0920869 | 0.11962899 |
| MYEF2 | 0.07959749 | 0.35159576 | −0.0364865 | 0.82385916 | 0.81070099 | 0.000042 |
| NCBP1 | 0.04849757 | 0.69951652 | 0.11261122 | 0.21837069 | 0.16554071 | 0.33464768 |
| NCBP2 | −0.0258423 | 0.98666122 | −0.0549593 | 0.91496419 | 0.59131465 | 0.150067 |
| NCL | −0.1092934 | 0.82857137 | −0.096881 | 0.962347 | 0.35134922 | 0.84438459 |
| NONO | 0.03729773 | 0.66413381 | 0.10903038 | 0.59509419 | 0.22861473 | 0.13064693 |
| NOSIP | −0.1174181 | 0.24280862 | −0.1117293 | 0.94155346 | 0.67773746 | 0.00145799 |
| NOVA1 | 0.08411776 | 0.64149828 | 0.04693805 | 0.53531439 | 0.75969812 | 0.00230848 |
| NOVA2 | 0.14266572 | 0.00634371 | 0.0052168 | 0.48439208 | 0.77657004 | 0.06144021 |
| NSRP1 | −0.0629648 | 0.86242429 | −0.0443106 | 0.83014475 | 0.62229957 | 0.00770638 |
| PABPN1 | 0.22378069 | 0.03279375 | 0.19990587 | 0.00033075 | 0.075512 | 0.0000982 |
| PAXBP1 | 0.01347077 | 0.63200801 | 0.0588208 | 0.38956843 | 0.61377926 | 0.25931165 |
| PCBP1 | 0.1996066 | 0.25053532 | 0.11991819 | 0.24401239 | 0.48065473 | 0.79644508 |
| PCBP4 | 0.10568851 | 0.33749431 | 0.14473313 | 0.02055266 | 0.32880747 | 0.64631277 |
| PHF5A | −0.0161103 | 0.88898191 | −0.1732668 | 0.69420747 | 0.34800504 | 0.8581711 |
| PHPT1 | −0.031664 | 0.96418695 | 0.01927215 | 0.56931211 | 0.54854992 | 0.37733256 |
| PLRG1 | −0.0546561 | 0.83852692 | −0.0115438 | 0.64710093 | 0.9772835 | 0.00170283 |
| PNN | 0.50353214 | 0.00187306 | 0.34701008 | 0.00000694 | 0.41031531 | 0.8271718 |
| POLDIP3 | 0.08024517 | 0.03986434 | 0.02155762 | 0.16783127 | 0.40517323 | 0.86978836 |
| POLR2A | 0.06288636 | 0.622028 | 0.09432741 | 0.38425393 | 0.49000168 | 0.52430346 |
| PPARGC1A | 0.0603738 | 0.7945088 | 0.16820238 | 0.20287911 | 0.24873378 | 0.44097702 |
| PPIE | 0.06146908 | 0.55297429 | 0.08798684 | 0.05379319 | 0.4238703 | 0.88897875 |
| PPIG | 0.2138375 | 0.24280862 | 0.16953109 | 0.12078002 | 0.4148038 | 0.68948484 |
| PPIH | 0.01189168 | 0.52229982 | 0.07744064 | 0.1976214 | 1.00551789 | 0.1525501 |
| PPIL1 | −0.0358945 | 1 | −0.0834696 | 0.99573033 | 0.57773534 | 0.11340497 |
| PPIL2 | −0.075507 | 0.55297429 | −0.2067892 | 0.21479538 | 0.51964158 | 0.21630942 |
| PPIL3 | −0.0388364 | 1 | −0.1131253 | 0.92984994 | 0.3725468 | 0.95634055 |
| PPIL4 | −0.0578549 | 0.78072627 | 0.01865145 | 0.67922127 | 0.50746721 | 0.27435345 |
| PPP1R8 | 0.06962363 | 0.48349462 | 0.13440209 | 0.55555731 | 0.52531162 | 0.56764202 |
| PPP2CA | −0.1336378 | 0.66413381 | −0.1012725 | 0.92256232 | 0.42982169 | 0.76820952 |
| PPP2R1A | 0.03081246 | 0.71659089 | 0.02237961 | 0.68749154 | 0.1546274 | 0.42009614 |
| PPP4R2 | −0.123351 | 0.35385117 | 0.00756772 | 0.38756349 | 0.26023438 | 0.42009614 |
| PPWD1 | 0.22337441 | 0.20598173 | 0.09887589 | 0.39762866 | 0.48300227 | 0.21630942 |
| PQBP1 | 0.03548372 | 0.6205953 | −0.0523372 | 0.93821674 | 0.8975078 | 0.08340877 |
| PRCC | −0.0176239 | 0.75964005 | 0.01502063 | 0.71805898 | 0.86735992 | 0.02556896 |
| PRKRIP1 | 0.11675663 | 0.35385117 | 0.06182154 | 0.18314213 | 0.29942383 | 0.62251113 |
| PRMT5 | 0.10271645 | 0.30602287 | 0.04824181 | 0.64676527 | 0.10515082 | 0.12907191 |
| PRPF18 | 0.0510175 | 0.70538977 | 0.05504387 | 0.60711443 | 0.54391267 | 0.1194788 |
| PRPF19 | 0.14726037 | 0.51608925 | 0.19801347 | 0.45698587 | 0.4007874 | 0.8736286 |
| PRPF31 | 0.06291647 | 0.51608925 | −0.005262 | 0.24401239 | 0.352402 | 0.94817586 |
| PRPF38A | −0.0575529 | 0.98666122 | −0.0641946 | 0.9838681 | 0.97805943 | 0.00481484 |
| PRPF38B | −0.1272472 | 0.63200801 | −0.1194175 | 0.71779179 | 0.37900472 | 0.99444321 |
| PRPF39 | 0.27502082 | 0.20759505 | 0.26406138 | 0.08600664 | 1.34883135 | 0.00873374 |
| PRPF4 | 0.11822055 | 0.53741673 | −0.0141945 | 0.72864064 | 0.74027484 | 0.00211902 |
| PRPF40A | 0.00561675 | 0.86913163 | −0.0647738 | 0.95758029 | 0.61436797 | 0.01395188 |
| PRPF40B | 0.34913468 | 0.22918973 | 0.41147613 | 0.000087 | 1.01018005 | 0.0000102 |
| PRPF4B | 0.10431943 | 0.22262511 | 0.12388999 | 0.2642781 | 1.04400048 | 0.00000161 |
| PRPF6 | 0.03421124 | 0.61262392 | 0.0588698 | 0.55555731 | 0.28083683 | 0.64631277 |
| PSMB4 | −0.0463633 | 0.98666122 | 0.08763654 | 0.25867427 | 0.86249017 | 0.17493169 |
| PSME3 | −0.1159496 | 0.25968709 | 0.02381516 | 0.3071572 | 0.29813943 | 0.57648498 |
| PTBP1 | 0.10633751 | 0.33749431 | −0.0882168 | 0.98356888 | 0.52701814 | 0.28687604 |
| PTBP2 | 0.39913568 | 0.37396257 | 0.28858697 | 0.21426174 | 0.61808238 | 0.16111738 |
| PTBP3 | 0.08242489 | 0.39624519 | −0.0024849 | 0.56500908 | 0.24987126 | 0.56764202 |
| PUF60 | 0.20808371 | 0.09766465 | 0.27472801 | 0.08519461 | 0.61022015 | 0.11340497 |
| PUS1 | 0.0382738 | 0.64149828 | −0.0494328 | 0.92984994 | 0.34773556 | 0.8590006 |
| PUS7 | 0.09645063 | 0.01762907 | −0.0180126 | 0.74187989 | 0.47393014 | 0.72576848 |
| QKI | −0.0030988 | 0.9557438 | 0.09352796 | 0.55555731 | 1.10002205 | 0.00873374 |
| RBFOX1N- | 0.26854306 | 0.00032314 | 1.14396438 | 2.08E−10 | 1.20306249 | 2.77E−09 |
| dCasRx-C | | | | | | |
| RBM10 | 0.00574792 | 0.63200801 | 0.05206665 | 0.44516488 | 0.77244536 | 0.27049816 |
| RBM11 | 0.31404319 | 0.00724878 | 0.59504734 | 0.00018567 | 1.20629237 | 0.00192484 |
| RBM14 | −0.0297971 | 0.98666122 | 0.03345721 | 0.76341107 | −0.2403192 | 0.0232861 |
| RBM15B | −0.1053017 | 0.39061691 | 0.56028247 | 0.06549404 | 0.28793863 | 0.45229853 |
| RBM17 | −0.0491094 | 0.98666122 | −0.0771289 | 1 | 1.18951479 | 0.02323052 |
| RBM19 | −0.0168329 | 0.88898191 | −0.1326949 | 0.92256232 | 0.15255216 | 0.2909114 |
| RBM23 | −0.1797086 | 0.09680977 | −0.0270127 | 0.58540742 | 0.78957279 | 0.150067 |
| RBM24 | −0.0478417 | 0.86492924 | 0.01580288 | 0.61838483 | 0.42294275 | 0.83767637 |
| RBM25 | 1.12266206 | 2.45E−14 | 1.2135481 | 1.7E−11 | 2.22821833 | 2.04E−15 |
| RBM28 | 0.03910968 | 0.68277778 | −0.0826541 | 1 | 0.09538851 | 0.23099986 |
| RBM3 | −0.0308485 | 0.98666122 | −0.0975711 | 0.90679361 | −0.1212961 | 0.000075 |
| RBM38 | 0.18325692 | 0.24280862 | 0.83142163 | 0.0000682 | 0.79207766 | 0.10422974 |
| RBM4 | −0.0806155 | 0.67838866 | 0.09581425 | 0.44473847 | 0.42919835 | 0.64631277 |
| RBM41 | 0.0941383 | 0.22918973 | 0.04133263 | 0.39762866 | 0.48543958 | 0.62216961 |
| RBM42 | 0.14330865 | 0.622028 | 0.07321728 | 0.55555731 | 0.42707688 | 0.64642705 |
| RBM4B | 0.22916235 | 0.39915598 | 0.23959457 | 0.11866535 | 0.53505687 | 0.66521886 |
| RBM5 | 0.03839566 | 0.09680977 | 0.08600273 | 0.19514483 | 0.83733276 | 0.11340497 |
| RBM6 | −0.2442864 | 0.39017084 | −0.5417162 | 0.1976214 | −0.2527796 | 0.02218676 |
| RBM7 | −0.008637 | 0.89078221 | −0.1582802 | 0.23897545 | 0.14403469 | 0.00499094 |
| RBM8A | −0.0935228 | 0.78804639 | 0.04548987 | 0.19862881 | 0.26047536 | 0.34739506 |
| RBMX | −0.5573423 | 0.20598173 | 0.16701749 | 0.04661621 | −0.2289241 | 0.1121416 |
| RBMX2 | −0.0383648 | 1 | 0.006294 | 0.24401239 | 1.20340589 | 0.08351342 |
| RBMXL2 | −0.424316 | 0.08500988 | −0.0595519 | 0.92904824 | 0.01578714 | 0.00567563 |
| RBMY1A1 | −0.109568 | 0.39017084 | 0.22300767 | 0.00189538 | 0.08485005 | 0.12907191 |
| RBMY1E | −0.1751663 | 0.38403312 | 0.69831306 | 0.00189538 | 0.15834441 | 0.49605578 |
| RBMY1F | −0.0502499 | 0.91202019 | 0.14017849 | 0.38435125 | 0.07413312 | 0.00102153 |
| RBPMS2 | 0.33064711 | 0.25053532 | 0.57699016 | 0.05702714 | 0.66615894 | 0.00156711 |
| RHEB | −0.1554475 | 0.37396257 | 0.01104671 | 0.24401239 | 0.7501144 | 0.09647979 |
| RNF113A | −0.0949687 | 0.44645536 | −0.0513893 | 0.90406956 | 0.39535535 | 0.9710889 |
| RNPC3 | 0.02226666 | 0.63200801 | −0.0150598 | 0.51118439 | 0.68256565 | 0.0910294 |
| RNPS1 | 0.29955636 | 0.22262511 | 0.28212282 | 0.0000226 | 1.51765752 | 8.5E−09 |
| RP9 | −0.0086532 | 0.94231579 | 0.02103999 | 0.24936587 | 0.34719313 | 0.80225835 |
| RPRD1B | 0.08645595 | 0.37396257 | 0.21436728 | 0.21780178 | 0.37398666 | 0.99444321 |
| RPSA | 0.08904826 | 0.00708542 | 0.14187403 | 0.20547751 | 0.69497313 | 0.41265816 |
| RPUSD4 | 0.05419861 | 0.63200801 | 0.1322638 | 0.18191183 | 0.13386624 | 0.00685661 |
| RRAGC | 0.02406126 | 0.53741673 | 0.11838551 | 0.05453227 | 0.34711231 | 0.87901239 |
| RSRC1 | 0.01284082 | 0.82322723 | 0.15749446 | 0.00188979 | 1.18423264 | 0.03275622 |
| RTCB | 0.10251561 | 0.57234463 | 0.11041934 | 0.00654674 | 0.49453015 | 0.13608468 |
| RTRAF | −0.0090496 | 0.94231579 | 0.00722113 | 0.63966648 | 0.58373565 | 0.36168257 |
| RUVBL2 | −0.105274 | 0.68277778 | −0.165562 | 0.70780602 | 0.07580654 | 0.04618854 |
| SAP18 | −0.0178022 | 0.9557438 | 0.0204444 | 0.67657824 | 0.54016625 | 0.29711616 |
| SAP30BP | 0.06986854 | 0.10823016 | 0.06957114 | 0.19514483 | 0.17539034 | 0.38037904 |
| SART1 | 0.06029638 | 0.54928808 | 0.07996757 | 0.47439371 | 0.17603442 | 0.11162208 |
| SART3 | 0.09006643 | 0.56939084 | −0.0588373 | 0.93821674 | −0.0069844 | 0.01863547 |
| SCAF11 | 0.00616401 | 0.83852692 | 0.17352002 | 0.00715016 | 0.05426479 | 0.33453888 |
| SCNM1 | 0.04222699 | 0.53741673 | 0.02328414 | 0.68744679 | 0.73463967 | 0.17976868 |
| SF1 | −0.2147224 | 0.53741673 | −0.1602197 | 0.58540742 | 1.1343593 | 0.01138343 |
| SF3A2 | −0.0709281 | 0.66785287 | −0.0571906 | 0.79881848 | 0.3829564 | 1 |
| SF3A3 | 0.1126971 | 0.63423892 | 0.01113423 | 0.39762866 | 0.06873614 | 0.11962899 |
| SF3B2 | 0.0488678 | 0.78718914 | −0.004211 | 0.71779179 | 0.04160919 | 0.00771527 |
| SF3B3 | 0.12467867 | 0.25053532 | 0.00348907 | 0.48190355 | 0.25761639 | 0.36400358 |
| SF3B4 | 0.26943301 | 0.32666924 | 0.28094066 | 0.33423948 | 0.68968232 | 0.00211902 |
| SF3B5 | 0.1384283 | 0.03279375 | 0.03290093 | 0.30840531 | 0.51798914 | 0.70456709 |
| SFPQ | −0.0814815 | 0.82322723 | −0.0638747 | 0.95524748 | 0.05720155 | 0.02795141 |
| SFSWAP | −0.0405332 | 1 | −0.0664735 | 0.93270579 | 0.68978061 | 0.06029074 |
| SLU7 | 0.06367472 | 0.64149828 | −0.0327544 | 0.50690437 | 0.26337999 | 0.11162208 |
| SMN1 | 0.06221579 | 0.17084255 | 0.24416891 | 0.02137183 | 1.03336044 | 0.00000852 |
| SMNDC1 | 0.07246914 | 0.55297429 | −0.0952796 | 0.93270579 | 0.66658634 | 0.21630942 |
| SMU1 | 0.01585355 | 0.82322723 | 0.06640675 | 0.55555731 | 0.57635168 | 0.16969424 |
| SNIP1 | 0.00213448 | 0.44645536 | 0.10532559 | 0.03591386 | 0.99587383 | 0.08840331 |
| SNRNP25 | 0.01248577 | 0.63371568 | 0.08064528 | 0.03591386 | 0.42466471 | 0.8736286 |
| SNRNP27 | −0.1456605 | 0.39335297 | −0.2026201 | 0.38756349 | 0.51271923 | 0.42009614 |
| SNRNP40 | 0.01472933 | 0.68277778 | −0.0357015 | 0.58508957 | 0.65704926 | 0.35889834 |
| SNRNP48 | −0.0244517 | 0.89123799 | 0.11294043 | 0.00561043 | 0.15625065 | 0.15586598 |
| SNRNP70 | 0.09936405 | 0.03279375 | 0.19952215 | 0.19512484 | 1.13313229 | 0.0385956 |
| SNRPA | 0.7718653 | 1.17E−06 | 0.75366874 | 4.75E−09 | 0.86167964 | 0.0000164 |
| SNRPA1 | 0.01260134 | 0.622028 | 0.00600264 | 0.68908518 | 0.37906386 | 0.99444321 |
| SNRPB | 0.05741926 | 0.6205953 | −0.0193779 | 0.50544863 | 0.56093367 | 0.10407979 |
| SNRPB2 | 0.04811506 | 0.55297429 | 0.05223866 | 0.58540742 | 0.51312535 | 0.41714944 |
| SNRPD1 | −0.0608893 | 0.96418695 | −0.0055354 | 0.77178027 | 0.12105861 | 0.07705102 |
| SNRPD2 | −0.0023712 | 0.91471006 | 0.2016829 | 0.08808 | 0.06826506 | 0.0608439 |
| SNRPD3 | 0.1313447 | 0.53741673 | 0.04946082 | 0.52373576 | 0.29483071 | 0.4535587 |
| SNRPE | 0.09283235 | 0.64149828 | −0.0679219 | 0.91454961 | 0.71332917 | 0.00063588 |
| SNRPF | 0.01054709 | 0.82322723 | −0.05281 | 0.93270579 | 0.51294512 | 0.30845912 |
| SNRPG | −0.0331116 | 1 | −0.0632779 | 0.95758029 | 0.69905192 | 0.15030412 |
| SNRPN | 0.00079342 | 0.8816967 | 0.06579169 | 0.54637289 | 0.59976169 | 0.40637223 |
| SNU13 | 0.05473373 | 0.52275689 | 0.0646763 | 0.12059638 | 0.494538 | 0.21630942 |
| SNW1 | −0.0641617 | 0.83547863 | −0.0779681 | 1 | 0.22056115 | 0.43061921 |
| SREK1 | −0.0112582 | 0.63200801 | 0.04094173 | 0.50231124 | 1.33624018 | 0.02792736 |
| SREK1IP1 | 0.0286192 | 0.98666122 | −0.0688638 | 0.95549643 | 0.12145594 | 0.26404033 |
| SRPK1 | 0.0990515 | 0.26876189 | 0.07440839 | 0.38435125 | 0.27108769 | 0.25408369 |
| SRPK2 | 0.15976868 | 0.29950177 | 0.10291426 | 0.12059638 | 0.43176282 | 0.89237038 |
| SRPK3 | −0.0410739 | 0.98666122 | −0.0432064 | 0.8173306 | 0.30360356 | 0.67196753 |
| SRRM4 | 0.08078887 | 0.20598173 | 0.13137292 | 0.46524821 | 0.56779227 | 0.23078127 |
| SRRT | −0.3673448 | 0.16143885 | −0.0310522 | 0.54685725 | 0.74481947 | 0.24514122 |
| SRSF1 | −0.2067867 | 0.51608925 | −0.2433096 | 0.50325379 | 0.58307183 | 0.03682257 |
| SRSF10 | 0.33786336 | 0.05695394 | 0.26550003 | 0.18192268 | 1.25034632 | 0.00806358 |
| SRSF11 | 0.10876005 | 0.26023147 | 0.15322452 | 0.23897545 | 1.4590479 | 0.00975743 |
| SRSF12 | 0.0095035 | 0.8816967 | −0.0350768 | 0.8173306 | 0.88485746 | 0.05840799 |
| SRSF2 | 0.21494329 | 0.30242453 | 0.07553083 | 0.2729989 | 0.61594563 | 0.07153401 |
| SRSF3 | 0.08098537 | 0.66065485 | 0.01637431 | 0.38435125 | 0.43786627 | 0.46073939 |
| SRSF4 | 0.14500461 | 0.22262511 | 0.58164127 | 0.04629189 | 0.87198547 | 0.0000127 |
| SRSF5 | 0.02273789 | 0.6243137 | 0.34207842 | 0.00000817 | 1.26891364 | 0.07705102 |
| SRSF6 | −0.1104929 | 0.63200801 | 0.5697782 | 0.02941074 | 0.87561771 | 0.05400043 |
| SRSF7 | −0.081999 | 0.68751975 | 0.14567956 | 0.02596802 | 0.61683837 | 0.00696535 |
| SRSF8 | 0.17331236 | 0.39017084 | 0.12576015 | 0.48190355 | 1.04222104 | 0.02218676 |
| SRSF9 | 0.01626664 | 0.8681652 | −0.0958308 | 0.95604878 | 0.23449331 | 0.05049578 |
| STRAP | 0.06738816 | 0.55297429 | 0.06060363 | 0.34433938 | 0.51774842 | 0.30218775 |
| SUGP1 | 0.03425827 | 0.64149828 | −0.005483 | 0.71779179 | 0.56572375 | 0.19750003 |
| SYF2 | 0.12589411 | 0.15763918 | 0.04376153 | 0.50836069 | 0.63903842 | 0.22555715 |
| SYNCRIP | 0.00177988 | 0.75919928 | −0.0127075 | 0.77484109 | 0.21879337 | 0.25706801 |
| TAF15 | 0.02428033 | 0.66413381 | 0.1782758 | 0.28738257 | −0.1844189 | 0.0000035 |
| TFIP11 | −0.0208656 | 0.96418695 | 0.12859378 | 0.27538646 | 0.2351931 | 0.34739506 |
| THOC1 | 0.02430865 | 0.63200801 | −0.093148 | 0.98932229 | 0.14523376 | 0.02323052 |
| THOC3 | 0.06021137 | 0.63200801 | 0.09162715 | 0.34433938 | 0.502713 | 0.23799868 |
| THOC5 | −0.024552 | 0.9557438 | −0.0227993 | 0.58640145 | 0.35050745 | 0.71686579 |
| THOC7 | 0.00367798 | 0.8507039 | 0.08609984 | 0.63966648 | 0.45551383 | 0.69581992 |
| THRAP3 | −0.2173054 | 0.44645536 | 0.11393273 | 0.54637289 | 0.50419456 | 0.33453888 |
| TIA1 | 0.03895736 | 0.622028 | 0.10702176 | 0.02676299 | 1.03328466 | 0.00000122 |
| TLE3 | 0.01795964 | 0.85479317 | 0.07272874 | 0.41509668 | 1.05034388 | 0.03011999 |
| TRA2A | 0.06290588 | 0.78625244 | −0.0843169 | 0.999795 | 0.4204694 | 0.8581711 |
| TRA2B | −0.0889261 | 0.83547863 | −0.1357442 | 0.74187989 | 0.49559884 | 0.175997 |
| TRPT1 | 0.04859019 | 0.77618493 | 0.00258633 | 0.8173306 | 0.89189091 | 0.01443308 |
| TSEN15 | 0.07861673 | 0.0129181 | 0.05478966 | 0.19514483 | 0.87367108 | 0.18715329 |
| TSEN2 | −0.1072287 | 0.24280862 | −0.0050568 | 0.53858068 | 0.57187334 | 0.64631277 |
| TSEN54 | 0.09829718 | 0.09680977 | 0.10026722 | 0.19514483 | 0.83999903 | 0.08910617 |
| TSSC4 | −0.0911535 | 0.53741673 | −0.06236 | 0.93946666 | 0.28573385 | 0.60905688 |
| TTF2 | 0.02937633 | 0.8273107 | −0.1720376 | 0.71016372 | 0.25196785 | 0.21630942 |
| TUBA1A | 0.05018072 | 0.65928553 | 0.07612691 | 0.20547751 | 0.14261573 | 0.11232706 |
| TUBB | 0.02222347 | 0.53741673 | 0.09619571 | 0.35195344 | −0.1018269 | 0.0000379 |
| TXNL4A | −0.0728203 | 0.62697244 | −0.1775567 | 0.58540742 | 0.40876721 | 0.89237038 |
| TXNL4B | −0.1112135 | 0.622028 | −0.1948357 | 0.63159199 | 0.79382834 | 0.07299007 |
| U2AF1 | −0.1619609 | 0.64149828 | −0.092448 | 0.97431847 | 0.57220523 | 0.2909114 |
| U2AF1L4 | 0.16180957 | 0.16101498 | 0.12534368 | 0.10142593 | 0.66424252 | 0.32739273 |
| U2AF2 | −0.2069908 | 0.17319512 | −0.1268145 | 0.68749154 | 0.40866646 | 0.84438459 |
| U2SURP | 0.06016247 | 0.39624519 | −0.0877338 | 0.99633225 | 0.43776834 | 0.68587621 |
| UBL5 | 0.06909313 | 0.62556676 | 0.04916114 | 0.53858068 | 0.53798887 | 0.67077068 |
| USB1 | 0.02428311 | 0.67838866 | −0.1495142 | 0.68749154 | 0.87027265 | 0.01253247 |
| USP39 | −0.1009165 | 0.39061691 | −0.1023523 | 0.93270579 | 0.2461756 | 0.54222206 |
| USP4 | 0.00873566 | 0.83852692 | 0.05235058 | 0.1976214 | 0.2047169 | 0.29267112 |
| USP49 | −0.0042248 | 0.68277778 | 0.0293256 | 0.44286055 | 0.09750009 | 0.44097702 |
| WAC | −0.3293019 | 0.17319512 | 0.17829313 | 0.01812399 | 0.30993658 | 0.68676103 |
| WBP11 | 0.07172043 | 0.6243137 | 0.00886503 | 0.63819888 | 0.64504582 | 0.05698696 |
| WBP4 | −0.0292841 | 0.98666122 | −0.0537256 | 0.94155346 | 0.85017931 | 0.21630942 |
| WDR83 | 0.09631179 | 0.27618095 | 0.012052 | 0.24401239 | 0.70408331 | 0.25408369 |
| WT1 | −0.0494372 | 0.93157524 | −0.0062017 | 0.51451598 | 0.67977997 | 0.2492565 |
| WTAP | −0.0426081 | 0.98666122 | 0.18735344 | 0.0585331 | 0.74353549 | 0.00926689 |
| YBX1 | −0.0314974 | 0.98666122 | −0.1108727 | 0.92256232 | −0.1527274 | 0.00156711 |
| YJU2 | −0.0478035 | 0.88618385 | −0.0675405 | 0.97431847 | 0.49863043 | 0.27777719 |
| ZBTB8OS | 0.02031131 | 0.82857137 | −0.0266705 | 0.58540742 | 0.59635283 | 0.42401474 |
| ZC3H11A | −0.1400805 | 0.55297429 | −0.0669237 | 0.93946666 | −0.1443876 | 0.19750003 |
| ZC3H14 | 0.24064173 | 0.2361468 | 0.16990145 | 0.02137183 | 0.01609669 | 0.12907191 |
| ZC3H18 | 0.16566171 | 0.44345017 | 0.11902531 | 0.05702714 | 0.96147269 | 0.0000957 |
| ZCCHC8 | 0.17161091 | 0.03462609 | 0.16020512 | 0.02791373 | 0.08671694 | 0.21057279 |
| ZCRB1 | 0.18508793 | 0.17084255 | 0.13956998 | 0.33423948 | 0.80590738 | 0.04016884 |
| ZFP36 | −0.0280869 | 0.98666122 | −0.099362 | 0.95604878 | 0.29176072 | 0.69581992 |
| ZMAT2 | 0.05879748 | 0.33749431 | 0.00583245 | 0.54685725 | 0.59518692 | 0.06371242 |
| ZMAT5 | −0.0384782 | 1 | −0.0118213 | 0.64710093 | 0.35103839 | 0.846357 |
| ZNF12 | 0.02619602 | 0.66413381 | 0.03341138 | 0.42633842 | 0.2932708 | 0.71686579 |
| ZNF326 | −0.103774 | 0.19861282 | −0.1244318 | 0.7566566 | 0.15697529 | 0.07800559 |
| ZNF830 | −0.0582849 | 0.8681652 | −0.0833301 | 0.99573033 | 0.52225322 | 0.12907191 |
| ZPR1 | 0.04331595 | 0.57619157 | 0.08929789 | 0.1976214 | 0.35240971 | 0.91135579 |
| ZRANB2 | −0.0861835 | 0.39061691 | 0.03395027 | 0.47439371 | 0.63435436 | 0.35388036 |
| ZRSR2 | 0.02725156 | 0.64149828 | 0.10650664 | 0.07919963 | 0.89993607 | 0.04879317 |

TABLE 3

| | | HEK293 | |
|---|---|---|---|
| | | T Gene | |
| Exon | | Expression | |
| targeted | Length | (RPKM) | Relevance |
| MAPT | 9 | 9 | Misregulation of tau exon 10 alternative splicing alters 3R-tau/4R-tau |
| exon | 3 | | balance and causes neurodegeneration in individuals with frontotemporal |
| 10 | | | dementia with Parkinsonism linked to chromosome 17 (FTDP-17) |
| CPEB4 | 2 | 3 | Skipping causes autism risk gene de-adenylation and autistic-like |
| microexon | 4 | | behaviours in mice (PMID: 30111840) |
| (exon 4) | | | |
| KRAS | 1 | 14 | Inclusion results in altered C-terminus of KRAS with different membrane- |
| exon 5 | 2 | | targeting sequences (PMID: 25561545). Inclusion isoform is enriched in |
| | 4 | | cancer stem-like cells (PMID: 34257283). |
| NUMA | 4 | 35 | Differentially spliced in breast cancer. Forced skipping by ASO causes |
| 1 exon | 2 | | decrease in MCF10A cell proliferation and centrosome amplification (PMID: |
| 16 | | | 27197215) |
| MEF2D | 2 | 6 | Skipped in autism spectrum disorders. Inclusion results in the introduction |
| microexon | 1 | | of a trans-activation domain that boosts the activity of MEF2D transcription |
| | | | factor (PMID: 15834131). |
| FGFR1 | 2 | 11 | Skipping of exon 3 results in FGFR1-beta isoform with higher binding affinity |
| exon 3 | 6 | | to FGF ligands than FGFR1-alpha (inclusion of exon 3). Overexpression of |
| | 7 | | FGFR1-beta is associated with tumorigenesis and poor survival in multiple |
| | | | tumors (PMID: 30713601). |
| HRAS | 8 | 47 | Inclusion results in altered C-terminus of HRAS, which alters its localization |
| exon 5 | 2 | | from the membrane to the cytosol and nucleus (PMID: 16436381). |
| CD46 | 9 | 29 | Previously targeted for repression by dCasRx (Nunez-Alvarez et al.). |
| exon 13 | 3 | | Differentially spliced in bladder cancer. Enforced expression of Exon 13- |
| | | | skipping isoform promoted, and Exon 13-containing isoform attenuated, |
| | | | cell growth, migration, and tumorigenicity in a xenograft model (PMID: |
| | | | 33767911). |
| PLOD2 | 6 | 24 | Previously targeted for repression by dCasRx (Nunez-Alvarez et al.). |
| exon 14 | 3 | | Inclusion of exon 13A encodes a loop that enhances UDP-glucose-binding |
| | | | in the GLT active site. CRISPR/Cas-9-mediated deletion of exon 13 A sharply |
| (AKA | | | reduces the growth and metastasis of LH2b-expressing LUADs in mice |
| exon 13a) | | | (PMID: 33875777). |
| SPAG9 | 3 | 31 | Previously targeted for repression by dCasRx (Nunez-Alvarez et al.). |
| exon 24 | 9 | | Differentially spliced in epithelial versus mesenchymal states of breast |
| | | | tumors (PMID: 32467311). |
| EVI5L | 3 | 6 | Previously targeted for repression by dCasRx (Nunez-Alvarez et al.). |
| exon 11 | 3 | | Differentially spliced in Vemurafenib-Resistant Melanoma Cells (PMID: |
| | | | 35883549). |
| FN1 | 2 | 7 | Inclusion of exon 25 (EDA exon) generates a fibronectin (FN1) isoform that |
| exon 25 | 7 | | is secreted to regulate cell adhesion and migration, whereas in the liver EDA |
| | 3 | | is skipped generating a soluble FN1 isoform that is secreted into plasma. |
| | | | Plasma FN1 constitutes the main protein components of the blood and is |
| | | | involved in blood clotting. (PMID: 28488700). |
| MDM4 | 6 | 10 | Exclusion results in the truncated MDMX-S isoform, containing only the p53 |
| exon 6 | 8 | | binding domain, which binds and inactivates p53 better than full-length |
| | | | MDMX (PMID: 12761890). |
| FLNB | 7 | 33 | Previously targeted for repression by dCasRx (Nunez-Alvarez et al.). The |
| exon 30 | 2 | | skipping of FLNB exon 30 induces epithelial-to-mesenchymal transition |
| | | | (EMT) by releasing the FOXC1 transcription factor. Moreover, skipping of |
| | | | FLNB exon 30 is strongly associated with EMT gene signatures in basal-like |
| | | | breast cancer patient samples (PMID: 30059005). |
| CASP2 | 6 | 35 | Inclusion results in a frameshift and production of a truncated caspase |
| exon 9 | 1 | | isoform (caspase-2S). Overexpression of the long isoform (caspase-2L) |
| | | | promotes cell death whereas caspase-2S antagonizes some apoptotic |
| | | | pathways (PMID: 8087842). |
| KIF21A | 2 | 10 | Previously targeted for activation by CREST system (PMID: 37395412) |
| exon 23 | 1 | | |

TABLE OF SEQUENCES

| SEQ | | |
|---|---|---|
| ID | | |
| NO | Name | Sequence |
| 1 | dCasRx- | MSPKKKRKVEASIEKKKSFAKGMGVKSTLVSGSKVYMTTFAEGSDARLEKIVEGDSIRSVNE |
| | RBM25 | GEAFSAEMADKNAGYKIGNAKFSHPKGYAVVANNPLYTGPVQQDMLGLKETLEKRYFGES |
| | | ADGNDNICIQVIHNILDIEKILAEYITNAAYAVNNISGLDKDIIGFGKFSTVYTYDEFKDPEHH |
| | | RAAFNNNDKLINAIKAQYDEFDNFLDNPRLGYFGQAFFSKEGRNYIINYGNECYDILALLSGL |
| | | AHWVVANNEEESRISRTWLYNLDKNLDNEYISTLNYLYDRITNELTNSFSKNSAANVNYIAE |
| | | TLGINPAEFAEQYFRFSIMKEQKNLGFNITKLREVMLDRKDMSEIRKNHKVFDSIRTKVYTM |
| | | MDFVIYRYYIEEDAKVAAANKSLPDNEKSLSEKDIFVINLRGSFNDDQKDALYYDEANRIWR |
| | | KLENIMHNIKEFRGNKTREYKKKDAPRLPRILPAGRDVSAFSKLMYALTMFLDGKEINDLLT |
| | | TLINKFDNIQSFLKVMPLIGVNAKFVEEYAFFKDSAKIADELRLIKSFARMGEPIADARRAMY |
| | | IDAIRILGTNLSYDELKALADTFSLDENGNKLKKGKHGMRNFIINNVISNKRFHYLIRYGDPA |
| | | HLHEIAKNEAVVKFVLGRIADIQKKQGQNGKNQIDRYYETCIGKDKGKSVSEKVDALTKIITG |
| | | MNYDQFDKKRSVIEDTGRENAEREKFKKIISLYLTVIYHILKNIVNINARYVIGFHCVERDAQL |
| | | YKEKGYDINLKKLEEKGFSSVTKLCAGIDETAPDKRKDVEKEMAERAKESIDSLESANPKLYA |
| | | NYIKYSDEKKAEEFTRQINREKAKTALNAYLRNTKWNVIIREDLLRIDNKTCTLFANKAVALE |
| | | VARYVHAYINDIAEVNSYFQLYHYIMQRIIMNERYEKSSGKVSEYFDAVNDEKKYNDRLLKL |
| | | LCVPFGYCIPRFKNLSIEALFDRNEAAKFDKEKKKVSGNSGSGPAAKRVKLDAAAYPYDVPD |
| | | YAGGRGGGGSGGGGSGGGGSGPAXXXXXXXXXMSFPPHLNRPPMGIPALPPGIPPPQFP |
| | | GFPPPVPPGTPMIPVPMSIMAPAPTVLVPTVSMVGKHLGARKDHPGLKAKENDENCGPT |
| | | TTVFVGNISEKASDMLIRQLLAKCGLVLSWKRVQGASGKLQAFGFCEYKEPESTLRALRLLH |
| | | DLQIGEKKLLVKVDAKTKAQLDEWKAKKKASNGNARPETVTNDDEEALDEETKRRDQMIK |
| | | GAIEVLIREYSSELNAPSQESDSHPRKKKKEKKEDIFRRFPVAPLIPYPLITKEDINAIEMEEDK |
| | | RDLISREISKFRDTHKKLEEEKGKKEKERQEIEKERRERERERERERERREREREREREREREKE |
| | | KERERERERDRDRDRTKERDRDRDRERDRDRDRERSSDRNKDRSRSREKSRDRERERERER |
| | | EREREREREREREREREREREREREREKDKKRDREEDEEDAYERRKLERKLREKEAAYQERLK |
| | | NWEIRERKKTREYEKEAEREEERRREMAKEAKRLKEFLEDYDDDRDDPKYYRGSALQKRLR |
| | | DREKEMEADERDRKREKEELEEIRQRLLAEGHPDPDAELQRMEQEAERRRQPQIKQEPESE |
| | | EEEEEKQEKEEKREEPMEEEEEPEQKPCLKPTLRPISSAPSVSSASGNATPNTPGDESPCGIII |
| | | PHENSPDQQQPEEHRPKIGLSLKLGASNSPGQPNSVKRKKLPVDSVFNKFEDEDSDDVPRK |
| | | RKLVPLDYGEDDKNATKGTVNTEEKRKHIKSLIEKIPTAKPELFAYPLDWSIVDSILMERRIRP |
| | | WINKKIIEYIGEEEATLVDFVCSKVMAHSSPQSILDDVAMVLDEEAEVFIVKMWRLLIYETEA |
| | | KKIGLVK |
| | | XXXXXXXXX can be TSLYKKVGT (SEQ ID NO: 9) or another sequence, each X can |
| | | be absent or present such that the sequence can be shorter. It can also be |
| | | longer. TSLYKKVGT is sequence that is included in the construct resulting |
| | | from the cloning strategy utilized. |
| 2 | RBFOX1N- | MNCEREQLRGNQEAAAAPDTMAQPYASAQFAPPQNGIPAEYTAPHPHPAPEYTGQTTV |
| | dCasRx- | PEHTLNLYPPAQTHSEQSPADTSAQTVSGTATQTDDAAPTDGQPQTQPSENTENKSQPK |
| | RBFOX1C | GGGGSGRASPKKKRKVEASIEKKKSFAKGMGVKSTLVSGSKVYMTTFAEGSDARLEKIVEG |
| | | DSIRSVNEGEAFSAEMADKNAGYKIGNAKFSHPKGYAVVANNPLYTGPVQQDMLGLKETL |
| | | EKRYFGESADGNDNICIQVIHNILDIEKILAEYITNAAYAVNNISGLDKDIIGFGKFSTVYTYDE |
| | | FKDPEHHRAAFNNNDKLINAIKAQYDEFDNFLDNPRLGYFGQAFFSKEGRNYIINYGNECY |
| | | DILALLSGLAHWVVANNEEESRISRTWLYNLDKNLDNEYISTLNYLYDRITNELTNSFSKNSA |
| | | ANVNYIAETLGINPAEFAEQYFRFSIMKEQKNLGFNITKLREVMLDRKDMSEIRKNHKVFDS |
| | | IRTKVYTMMDFVIYRYYIEEDAKVAAANKSLPDNEKSLSEKDIFVINLRGSFNDDQKDALYY |
| | | DEANRIWRKLENIMHNIKEFRGNKTREYKKKDAPRLPRILPAGRDVSAFSKLMYALTMFLD |
| | | GKEINDLLTTLINKFDNIQSFLKVMPLIGVNAKFVEEYAFFKDSAKIADELRLIKSFARMGEPI |
| | | ADARRAMYIDAIRILGTNLSYDELKALADTFSLDENGNKLKKGKHGMRNFIINNVISNKRFH |
| | | YLIRYGDPAHLHEIAKNEAVVKFVLGRIADIQKKQGQNGKNQIDRYYETCIGKDKGKSVSEK |
| | | VDALTKIITGMNYDQFDKKRSVIEDTGRENAEREKFKKIISLYLTVIYHILKNIVNINARYVIGF |
| | | HCVERDAQLYKEKGYDINLKKLEEKGFSSVTKLCAGIDETAPDKRKDVEKEMAERAKESIDSL |
| | | ESANPKLYANYIKYSDEKKAEEFTRQINREKAKTALNAYLRNTKWNVIIREDLLRIDNKTCTLF |
| | | ANKAVALEVARYVHAYINDIAEVNSYFQLYHYIMQRIIMNERYEKSSGKVSEYFDAVNDEKK |
| | | YNDRLLKLLCVPFGYCIPRFKNLSIEALFDRNEAAKFDKEKKKVSGNSGSGPKKKRKVAAAYP |
| | | YDVPDYAGGRGGGGSGGGGSGGGGSGPANATARVMTNKKTVNPYTNGWKLNPVVGA |
| | | VYSPEFYAGTVLLCQANQEGSSMYSAPSSLVYTSAMPGFPYPAATAAAAYRGAHLRGRGR |
| | | TVYNTFRAAAPPPPIPAYGGVVYQDGFYGADIYGGYAAYRYAQPTPATAAAYSDSYGRVYA |
| | | ADPYHHALAPAPTYGVGAMNAFAPLTDAKTRSHADDVGLVLSSLQASIYRGGYNRFAPY |
| 3 | dRfxCas13 | IEKKKSFAKGMGVKSTLVSGSKVYMTTFAEGSDARLEKIVEGDSIRSVNEGEAFSAEMADK |
| | d (dCasRx) | NAGYKIGNAKFSHPKGYAVVANNPLYTGPVQQDMLGLKETLEKRYFGESADGNDNICIQVI |
| | | HNILDIEKILAEYITNAAYAVNNISGLDKDIIGFGKFSTVYTYDEFKDPEHHRAAFNNNDKLIN |
| | | AIKAQYDEFDNFLDNPRLGYFGQAFFSKEGRNYIINYGNECYDILALLSGLAHWVVANNEEE |
| | | SRISRTWLYNLDKNLDNEYISTLNYLYDRITNELTNSFSKNSAANVNYIAETLGINPAEFAEQY |
| | | FRFSIMKEQKNLGFNITKLREVMLDRKDMSEIRKNHKVFDSIRTKVYTMMDFVIYRYYIEED |
| | | AKVAAANKSLPDNEKSLSEKDIFVINLRGSFNDDQKDALYYDEANRIWRKLENIMHNIKEFR |
| | | GNKTREYKKKDAPRLPRILPAGRDVSAFSKLMYALTMFLDGKEINDLLTTLINKFDNIQSFLK |
| | | VMPLIGVNAKFVEEYAFFKDSAKIADELRLIKSFARMGEPIADARRAMYIDAIRILGTNLSYD |
| | | ELKALADTFSLDENGNKLKKGKHGMRNFIINNVISNKRFHYLIRYGDPAHLHEIAKNEAVVK |
| | | FVLGRIADIQKKQGQNGKNQIDRYYETCIGKDKGKSVSEKVDALTKIITGMNYDQFDKKRS |
| | | VIEDTGRENAEREKFKKIISLYLTVIYHILKNIVNINARYVIGFHCVERDAQLYKEKGYDINLKKL |
| | | EEKGFSSVTKLCAGIDETAPDKRKDVEKEMAERAKESIDSLESANPKLYANYIKYSDEKKAEE |
| | | FTRQINREKAKTALNAYLRNTKWNVIIREDLLRIDNKTCTLFANKAVALEVARYVHAYINDIA |
| | | EVNSYFQLYHYIMQRIIMNERYEKSSGKVSEYFDAVNDEKKYNDRLLKLLCVPFGYCIPRFKN |
| | | LSIEALFDRNEAAKFDKEKKKVSGNS |
| 4 | RBM25 | MSFPPHLNRPPMGIPALPPGIPPPQFPGFPPPVPPGTPMIPVPMSIMAPAPTVLVPTVSM |
| | | VGKHLGARKDHPGLKAKENDENCGPTTTVFVGNISEKASDMLIRQLLAKCGLVLSWKRVQ |
| | | GASGKLQAFGFCEYKEPESTLRALRLLHDLQIGEKKLLVKVDAKTKAQLDEWKAKKKASNG |
| | | NARPETVTNDDEEALDEETKRRDQMIKGAIEVLIREYSSELNAPSQESDSHPRKKKKEKKEDI |
| | | FRRFPVAPLIPYPLITKEDINAIEMEEDKRDLISREISKFRDTHKKLEEEKGKKEKERQEIEKERR |
| | | ERERERERERERREREREREREREREKEKERERERERDRDRDRTKERDRDRDRERDRDRDR |
| | | ERSSDRNKDRSRSREKSRDREREREREREREREREREREREREREREREREREREKDKKRDR |
| | | EEDEEDAYERRKLERKLREKEAAYQERLKNWEIRERKKTREYEKEAEREEERRREMAKEAKR |
| | | LKEFLEDYDDDRDDPKYYRGSALQKRLRDREKEMEADERDRKREKEELEEIRQRLLAEGHP |
| | | DPDAELORMEQEAERRRQPQIKQEPESEEEEEEKQEKEEKREEPMEEEEEPEQKPCLKPTL |
| | | RPISSAPSVSSASGNATPNTPGDESPCGIIIPHENSPDQQQPEEHRPKIGLSLKLGASNSPGQ |
| | | PNSVKRKKLPVDSVFNKFEDEDSDDVPRKRKLVPLDYGEDDKNATKGTVNTEEKRKHIKSLI |
| | | EKIPTAKPELFAYPLDWSIVDSILMERRIRPWINKKIIEYIGEEEATLVDFVCSKVMAHSSPQSI |
| | | LDDVAMVLDEEAEVFIVKMWRLLIYETEAKKIGLVKL |
| | | *underlined sequence in italics corresponds to C-terminal domain |
| 5 | NLS | PKKKRKV |
| 6 | NLS | PAAKRVKLD |
| 7 | linker | GGRGGGGGGGGSGGGGSGPA |
| 8 | PWI | PELFAYPLDWSIVDSILMERRIRPWINKKIIEYIGEEEATLVDFVCSKVMAHSSPQSILDDVA |
| | | MVLDEEAEVFIVKMWRLLIYETEAKKIGLVK |
| 9 | extra | XXXXXXXXX optionally TSLYKKVGT |

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### CITATIONS FOR REFERENCES REFERRED TO IN THE SPECIFICATION

1 Pan, Q., Shai, O., Lee, L. J., Frey, B. J. & Blencowe, B. J. Deep surveying of alternative splicing complexity in the human transcriptome by high-throughput sequencing. Nature Genetics 40, 1413-1415, doi:10.1038/ng.259 (2008).
2 Wang, E. T. et al. Alternative isoform regulation in human tissue transcriptomes. Nature 456, 470-476, doi:10.1038/nature07509 (2008).
3 Sinitcyn, P. et al. Global detection of human variants and isoforms by deep proteome sequencing. Nat Biotechnol, doi:10.1038/s41587-023-01714-x (2023).
4 Baralle, F. E. & Giudice, J. Alternative splicing as a regulator of development and tissue identity. Nat Rev Mol Cell Biol 18, 437-451, doi:10.1038/nrm.2017.27 (2017).
5 Ule, J. & Blencowe, B. J. Alternative Splicing Regulatory Networks: Functions, Mechanisms, and Evolution. Mol Cell 76, 329-345, doi:10.1016/j.molcel.2019.09.017 (2019).
6 Rogalska, M. E., Vivori, C. & Valcarcel, J. Regulation of pre-mRNA splicing: roles in physiology and disease, and therapeutic prospects. Nat Rev Genet, doi:10.1038/s41576-022-00556-8 (2022).
7 Marasco, L. E. & Kornblihtt, A. R. The physiology of alternative splicing. Nat Rev Mol Cell Biol, doi:10.1038/s41580-022-00545-z (2022).
8 Scotti, M. M. & Swanson, M. S. RNA mis-splicing in disease. Nat Rev Genet 17, 19-32, doi:10.1038/nrg.2015.3 (2016).
9 Lord, J. & Baralle, D. Splicing in the Diagnosis of Rare Disease: Advances and Challenges. Front Genet 12, 689892, doi:10.3389/fgene.2021.689892 (2021).
10 Sergeeva, O. V., Shcherbinina, E. Y., Shomron, N. & Zatsepin, T. S. Modulation of RNA Splicing by Oligonucleotides: Mechanisms of Action and Therapeutic Implications. Nucleic Acid Ther 32, 123-138, doi:10.1089/nat.2021.0067 (2022).
11 Rigo, F., Hua, Y., Krainer, A. R. & Bennett, C. F. Antisense-based therapy for the treatment of spinal muscular atrophy. J Cell Biol 199, 21-25, doi:10.1083/jcb.201207087 (2012).
12 Gonatopoulos-Pournatzis, T. et al. Genetic interaction mapping and exon-resolution functional genomics with a hybrid Cas9-Cas12a platform. Nat Biotechnol 38, 638-648, doi:10.1038/s41587-020-0437-z (2020).
13 Thomas, J. D. et al. RNA isoform screens uncover the essentiality and tumor-suppressor activity of ultraconserved poison exons. Nat Genet 52, 84-94, doi:10.1038/s41588-019-0555-z (2020).
14 Wang, Y., Cheong, C. G., Hall, T. M. & Wang, Z. Engineering splicing factors with designed specificities. Nat Methods 6, 825-830, doi:10.1038/nmeth. 1379 (2009).
15 Zhao, Y. Y. et al. Expanding RNA binding specificity and affinity of engineered PUF domains. Nucleic Acids Res 46, 4771-4782, doi:10.1093/nar/gkyl34 (2018).
16 Fernandez Alanis, E. et al. An exon-specific U1 small nuclear RNA (snRNA) strategy to correct splicing defects. Hum Mol Genet 21, 2389-2398, doi:10.1093/hmg/dds045 (2012).
17 Gaur, R. K. RNA interference: a potential therapeutic tool for silencing splice isoforms linked to human diseases. Biotechniques Suppl, 15-22, doi:10.2144/000112165 (2006).
18 Schneider-Poetsch, T., Chhipi-Shrestha, J. K. & Yoshida, M. Splicing modulators: on the way from nature to clinic. J Antibiot (Tokyo) 74, 603-616, doi:10.1038/s41429-021-00450-1 (2021).
19 van Beljouw, S. P. B., Sanders, J., Rodriguez-Molina, A. & Brouns, S. J. J. RNA-targeting CRISPR-Cas systems. Nat Rev Microbiol 21, 21-34, doi:10.1038/s41579-022-00793-y (2023).
20 Konermann, S. et al. Transcriptome Engineering with RNA-Targeting Type VI-D CRISPR Effectors. Cell 173, 665-676 e614, doi:10.1016/j.cell.2018.02.033 (2018).
21 Du, M., Jillette, N., Zhu, J. J., Li, S. & Cheng, A. W. CRISPR artificial splicing factors. Nat Commun 11, 2973, doi:10.1038/s41467-020-16806-4 (2020).
22 Núñez-Álvarez, Y., Espie-Caullet, T. & Luco, R. F. A CRISPR-dCas13 RNA-editing tool to study alternative splicing. bioRxiv, doi:https://doi.org/10.1101/2022.05.24.493209.
23 Leclair, N. K. et al. Poison Exon Splicing Regulates a Coordinated Network of SR Protein Expression during Differentiation and Tumorigenesis. Mol Cell 80, 648-665 e649, doi:10.1016/j.molcel.2020.10.019 (2020).
24 Irimia, M. et al. A highly conserved program of neuronal microexons is misregulated in autistic brains. Cell 159, 1511-1523, doi:10.1016/j.cell.2014.11.035 (2014).
25 Gonatopoulos-Pournatzis, T. et al. Genome-wide CRISPR-Cas9 Interrogation of Splicing Networks Reveals a Mechanism for Recognition of Autism-Misregulated Neuronal Microexons. Mol Cell 72, 510-524 e512, doi:10.1016/j.molcel.2018.10.008 (2018).
26 Lorson, C. L., Hahnen, E., Androphy, E. J. & Wirth, B. A single nucleotide in the SMN gene regulates splicing and is responsible for spinal muscular atrophy. Proc Natl Acad Sci U S A 96, 6307-6311 (1999).
27 Best, A. J. et al. High-throughput sensitive screening of small molecule modulators of microexon alternative splicing using dual Nano and Firefly luciferase reporters. Submitted (2023).
28 Agafonov, D. E. et al. Semiquantitative proteomic analysis of the human spliceosome via a novel two-dimensional gel electrophoresis method. Mol Cell Biol 31, 2667-2682, doi:10.1128/MCB.05266-11 (2011).
29 Kastner, B., Will, C. L., Stark, H. & Luhrmann, R. Structural Insights into Nuclear pre-mRNA Splicing in Higher Eukaryotes. Cold Spring Harb Perspect Biol 11, doi:10.1101/cshperspect.a032417 (2019).
30 Dopie, J., Sweredoski, M. J., Moradian, A. & Belmont, A. S. Tyramide signal amplification mass spectrometry (TSA-MS) ratio identifies nuclear speckle proteins. J Cell Biol 219, doi:10.1083/jcb.201910207 (2020).
31 Yang, X. et al. A public genome-scale lentiviral expression library of human ORFs. Nat Methods 8, 659-661, doi:10.1038/nmeth. 1638 (2011).
32 Gottschalk, A. et al. A comprehensive biochemical and genetic analysis of the yeast U1 snRNP reveals five novel proteins. RNA 4, 374-393 (1998).
33 Li, X. et al. CryoEM structure of Saccharomyces cerevisiae U1 snRNP offers insight into alternative splicing. Nat Commun 8, 1035, doi:10.1038/s41467-017-01241-9 (2017).
34 Fortes, P. et al. Identification and characterization of RED120: a conserved PWI domain protein with links to splicing and 3′-end formation. FEBS Lett 581, 3087-3097, doi:10.1016/j.febslet.2007.05.066 (2007).
35 Carlson, S. M. et al. RBM25 is a global splicing factor promoting inclusion of alternatively spliced exons and is itself regulated by lysine mono-methylation. J Biol Chem 292, 13381-13390, doi:10.1074/jbc.M117.784371 (2017).
36 Wessels, H. H. et al. Massively parallel Cas13 screens reveal principles for guide RNA design. Nat Biotechno/38, 722-727, doi:10.1038/s41587-020-0456-9 (2020).
37 Guo, X. et al. Transcriptome-wide Cas13 guide RNA design for model organisms and viral RNA pathogens. Cell Genom 1, doi:10.1016/j.xgen.2021.100001 (2021).
38 Liu, F. & Gong, C. X. Tau exon 10 alternative splicing and tauopathies. Mol Neurodegener 3, 8, doi:10.1186/1750-1326-3-8 (2008).
39 Parras, A. et al. Autism-like phenotype and risk gene mRNA deadenylation by CPEB4 mis-splicing. Nature 560, 441-446, doi:10.1038/s41586-018-0423-5 (2018).
40 Liu, Z., Jillette, N., Robson, P. & Cheng, A. W. Simultaneous multifunctional transcriptome engineering by CRISPR RNA scaffold. Nucleic Acids Res 51, e77, doi:10.1093/nar/gkad547 (2023).
41 Kluesner, M. G. et al. CRISPR-Cas9 cytidine and adenosine base editing of splice-sites mediates highly-efficient disruption of proteins in primary and immortalized cells. Nat Commun 12, 2437, doi:10.1038/s41467-021-22009-2 (2021).
42 Gapinske, M. et al. CRISPR-SKIP: programmable gene splicing with single base editors. Genome Biol 19, 107, doi:10.1186/s13059-018-1482-5 (2018).
43 Zhang, C. et al. Structural Basis for the RNA-Guided Ribonuclease Activity of CRISPR-Cas13d. Cell 175, 212-223 e217, doi:10.1016/j.cell.2018.09.001 (2018).Nunez-Alvarez et al. doi:https://doi.org/10.1101/2022.05.24.493209
44. Schmok, JC., and Yeo, GW. Strategies for programmable manipulation of alternative splicing. Current Opinion in Genetics & Development 89 https://doi.org/10.1016/j.gde.2024.102272

1. A method of modulating splicing in a cell, comprising introducing into the cell:
a fusion protein comprising a catalytically deactivated Cas13d (dCas13d) and a splicing factor, wherein the splicing factor is RBM25 polypeptide, or a nucleic acid or vector encoding said fusion protein; and
one or more guide RNAs,
wherein the one or more guide RNAs targets an intron downstream of an exon of a first pre-mRNA.

2. The method of claim 1, wherein the dCas13d is catalytically deactivated dCas13d from Ruminococcus flavefaciens XPD3002 (dCasRx); wherein the RBM25 polypeptide comprises an amino acid sequence of at least 90% or at least 95% sequence identity to SEQ ID NO: 4; wherein the RBM25 polypeptide is a fragment lacking the PWI domain or the Cterminus, optionally comprising at least amino acids 1-610 of SEQ ID NO: 4; and/or wherein the dCas13d is linked to one or more nuclear localization signals, optionally comprising the sequence of SEQ ID NO: 5 or SEQ ID NO: 6.

3. The method of claim 2, wherein the dCasRx comprises an amino acid sequence of at least 90% or at least 95% sequence identity to SEQ ID NO: 3.

4.-6. (canceled)

7. The method of claim 1, wherein the RBM25 polypeptide is fused to the N-terminus of dCasRx or wherein the RBM25 polypeptide is fused to the C-terminus of dCasRx.

8. (canceled)

9. The method of claim 1, further comprising a linker, optionally wherein the linker is a glycine-serine linker, optionally comprising an amino acid sequence of at least 90% or at least 95% sequence identity of SEQ ID NO: 7.

10. (canceled)

11. The method of claim 1, wherein the nucleic acid comprises a sequence that is operably linked to one or more regulatory elements and/or wherein the nucleic acid or vector is introduced into the cell.

12. (canceled)

13. The method of claim 1, wherein the vector is a viral vector, wherein the viral vector is an adeno-associated viral vector (AAV), an adenoviral vector, a retroviral vector, a transposon vector or a lentiviral vector.

14. (canceled)

15. The method of claim 1, wherein the one or more guide RNAs is three guide RNAs, two guide RNAs, or preferably one guide RNA or wherein the one of more guide RNAs is an array of guide RNAs.

16. (canceled)

17. The method of claim 1, further comprising introducing into the cell one or more additional guide RNAs targeting a second intron downstream of a second exon of the first pre-mRNA; further comprising introducing into the cell one or more additional guide RNAs targeting a second intron downstream of a second exon of a second pre-mRNA; further comprising introducing into the cell one or more additional guide RNAs targeting a second exon of the first pre-mRNA and/or further comprising introducing into the cell one or more additional guide RNAs targeting a second exon of a second pre-mRNA.

18.-20. (canceled)

21. The method of claim 1, wherein the one or more guide RNAs and the one or more additional guide RNAs are provided in one or more arrays of guide RNAs.

22. A fusion protein comprising a catalytically deactivated Cas13d (dCas13d) and a splicing factor, wherein the splicing factor is RBM25 polypeptide.

23. The fusion protein of claim 22, wherein the dCas13d is catalytically deactivated dCas13d from Ruminococcus flavefaciens XPD3002 (dCasRx);
wherein the RBM25 polypeptide comprises an amino acid sequence of at least 90% or at least 95% sequence identity to SEQ ID NO: 4; wherein the RBM25 polypeptide is a fragment lacking the PWI domain or the Cterminus, optionally comprising at least amino acids 1-610 of SEQ ID NO: 4, and/or wherein the dCas13d is linked to one or more nuclear localization signals, optionally comprising the sequence of SEQ ID NO: 5 or SEQ ID NO: 6.

24. The fusion protein of claim 23, wherein the dCasRx comprises an amino acid sequence of at least 90% or at least 95% sequence identity to SEQ ID NO: 3.

25.-27. (canceled)

28. The fusion protein of claim 22, wherein the RBM25 polypeptide is fused to the N-terminus of dCasRx or wherein the RBM25 polypeptide is fused to the C-terminus of dCasRx.

29. (canceled)

30. The fusion protein of claim 22, further comprising a linker, optionally wherein the linker is a glycine-serine linker, optionally comprising an amino acid sequence of at least 90% or at least 95% sequence identity of SEQ ID NO:.

31. (canceled)

32. A nucleic acid comprising a polynucleotide sequence encoding the fusion protein of claim 22, optionally wherein the sequence is operably linked to one or more regulatory elements.

33. A vector comprising the nucleic acid of claim 32.

34. The vector of claim 33, wherein the vector is a viral vector, wherein the viral vector is an adeno-associated viral vector (AAV), an adenoviral vector, a retroviral vector, a transposon vector or a lentiviral vector.

35. (canceled)

36. A recombinant cell comprising the nucleic acid claim 32 or a vector comprising said nucleic acid.

37. A composition comprising the fusion protein of claim 22, a nucleic acid encoding the fusion protein, a vector comprising the nucleic acid, or a recombinant cell comprising the nucleic acid or vector, and optionally a pharmaceutically acceptable carrier.

38. A kit comprising: the fusion protein of claim 22. a nucleic acid encoding the fusion protein, a vector comprising the nucleic acid, a recombinant cell comprising the nucleic acid or vector, or a composition comprising the fusion protein, the nucleic acid, the vector, the recombinant cell, and optionally a nucleic acid comprising a sequence encoding one or more guide RNAs.


SEQUENCE LISTING (9 sequences)

SEQ ID NO: 1 - protein, 1875 aa, synthetic construct
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPAAKRVKLDAAAYPYDVPDYAGGRGGGGSGGGGSGGGGSGPAXXXXXXXXXMSFPPHLNRPPMGIPALPPGIPPPQFPGFPPPVPPGTPMIPVPMSIMAPAPTVLVPTVSMVGKHLGARKDHPGLKAKENDENCGPTTTVFVGNISEKASDMLIRQLLAKCGLVLSWKRVQGASGKLQAFGFCEYKEPESTLRALRLLHDLQIGEKKLLVKVDAKTKAQLDEWKAKKKASNGNARPETVTNDDEEALDEETKRRDQMIKGAIEVLIREYSSELNAPSQESDSHPRKKKKEKKEDIFRRFPVAPLIPYPLITKEDINAIEMEEDKRDLISREISKFRDTHKKLEEEKGKKEKERQEIEKERRERERERERERERREREREREREREREKEKERERERERDRDRDRTKERDRDRDRERDRDRDRERSSDRNKDRSRSREKSRDREREREREREREREREREREREREREREREREREREKDKKRDREEDEEDAYERRKLERKLREKEAAYQERLKNWEIRERKKTREYEKEAEREEERRREMAKEAKRLKEFLEDYDDDRDDPKYYRGSALQKRLRDREKEMEADERDRKREKEELEEIRQRLLAEGHPDPDAELQRMEQEAERRRQPQIKQEPESEEEEEEKQEKEEKREEPMEEEEEPEQKPCLKPTLRPISSAPSVSSASGNATPNTPGDESPCGIIIPHENSPDQQQPEEHRPKIGLSLKLGASNSPGQPNSVKRKKLPVDSVFNKFEDEDSDDVPRKRKLVPLDYGEDDKNATKGTVNTEEKRKHIKSLIEKIPTAKPELFAYPLDWSIVDSILMERRIRPWINKKIIEYIGEEEATLVDFVCSKVMAHSSPQSILDDVAMVLDEEAEVFIVKMWRLLIYETEAKKIGLVK

SEQ ID NO: 2 - protein, 1353 aa, synthetic construct
MNCEREQLRGNQEAAAAPDTMAQPYASAQFAPPQNGIPAEYTAPHPHPAPEYTGQTTVPEHTLNLYPPAQTHSEQSPADTSAQTVSGTATQTDDAAPTDGQPQTQPSENTENKSQPKGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPANATARVMTNKKTVNPYTNGWKLNPVVGAVYSPEFYAGTVLLCQANQEGSSMYSAPSSLVYTSAMPGFPYPAATAAAAYRGAHLRGRGRTVYNTFRAAAPPPPIPAYGGVVYQDGFYGADIYGGYAAYRYAQPTPATAAAYSDSYGRVYAADPYHHALAPAPTYGVGAMNAFAPLTDAKTRSHADDVGLVLSSLQASIYRGGYNRFAPY

SEQ ID NO: 3 - protein, 966 aa, synthetic construct
IEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 4 - protein, 844 aa, Homo sapiens
MSFPPHLNRPPMGIPALPPGIPPPQFPGFPPPVPPGTPMIPVPMSIMAPAPTVLVPTVSMVGKHLGARKDHPGLKAKENDENCGPTTTVFVGNISEKASDMLIRQLLAKCGLVLSWKRVQGASGKLQAFGFCEYKEPESTLRALRLLHDLQIGEKKLLVKVDAKTKAQLDEWKAKKKASNGNARPETVTNDDEEALDEETKRRDQMIKGAIEVLIREYSSELNAPSQESDSHPRKKKKEKKEDIFRRFPVAPLIPYPLITKEDINAIEMEEDKRDLISREISKFRDTHKKLEEEKGKKEKERQEIEKERRERERERERERERREREREREREREREKEKERERERERDRDRDRTKERDRDRDRERDRDRDRERSSDRNKDRSRSREKSRDREREREREREREREREREREREREREREREREREREKDKKRDREEDEEDAYERRKLERKLREKEAAYQERLKNWEIRERKKTREYEKEAEREEERRREMAKEAKRLKEFLEDYDDDRDDPKYYRGSALQKRLRDREKEMEADERDRKREKEELEEIRQRLLAEGHPDPDAELQRMEQEAERRRQPQIKQEPESEEEEEEKQEKEEKREEPMEEEEEPEQKPCLKPTLRPISSAPSVSSASGNATPNTPGDESPCGIIIPHENSPDQQQPEEHRPKIGLSLKLGASNSPGQPNSVKRKKLPVDSVFNKFEDEDSDDVPRKRKLVPLDYGEDDKNATKGTVNTEEKRKHIKSLIEKIPTAKPELFAYPLDWSIVDSILMERRIRPWINKKIIEYIGEEEATLVDFVCSKVMAHSSPQSILDDVAMVLDEEAEVFIVKMWRLLIYETEAKKIGLVKL

SEQ ID NO: 5 - protein, 7 aa, simiam virus 40
PKKKRKV

SEQ ID NO: 6 - protein, 9 aa, Homo sapiens
PAAKRVKLD

SEQ ID NO: 7 - protein, 21 aa, synthetic construct
GGRGGGGSGGGGSGGGGSGPA

SEQ ID NO: 8 - protein, 94 aa, Homo sapiens
PELFAYPLDWSIVDSILMERRIRPWINKKIIEYIGEEEATLVDFVCSKVMAHSSPQSILDDVAMVLDEEAEVFIVKMWRLLIYETEAKKIGLVK

SEQ ID NO: 9 - protein, 9 aa, synthetic construct
TSLYKKVGT